(19) United States
(12) Patent Application Publication
(10) Pub. No.: US 2026/0364211 A1
(43) Pub. Date: Nov. 19, 2026

(54) DETECTION OF SOLID TUMOR CANCERS WITH SELF-AGGLOMERATING NANOHYDROGELS (SANGS)

(71) Applicant: Georgia Tech Research Corporation, Atlanta, GA (US)

(72) Inventors: Stephen N. Housley, Atlanta, GA (US); M.G. Finn, Atlanta, GA (US)

(21) Appl. No.: 19/458,019

(22) Filed: Jan. 23, 2026

Related U.S. Application Data
(60) Provisional application No. 63/749,290, filed on Jan. 24, 2025.
(60) Provisional application No. 63/749,295, filed on Jan. 24, 2025.

(51) Int. Cl. A61K 49/00; G01N 21/64

(52) U.S. Cl. CPC A61K 49/0073; A61K 49/0017; G01N 21/6428; G01N 2021/6439

(57) ABSTRACT

Delivery of agents to tumor microenvironments is challenging due to their rapid clearance, susceptibility to enzymatic cleavage, and inability to traverse plasma membranes, impeding effective imaging and detection of tumors associated with a variety of disorders. Described herein is a self-agglomerating nanohydrogel and methods of delivering imaging agents, as well as methods of detecting tumors with the nanohydrogel, such as solid tumors.

### CROSS-REFERENCE TO RELATED APPLICATIONS

This application claims the benefit of U.S. Provisional Patent Application No. 63/749,290, filed Jan. 24, 2025, and U.S. Provisional Patent Application No. 63/749,295, filed Jan. 24, 2025, each of which is incorporated by reference herein in its entirety.

### BACKGROUND

Nanotechnology has revolutionized the field of diagnostics, addressing long-standing challenges in sensitivity, specificity, and multiplexing. Traditional diagnostic methods, such as imaging and histopathology, often detect diseases only after significant progression, limiting therapeutic effectiveness. Nanotechnology-based diagnostic tools overcome these barriers by leveraging the unique properties of nanoscale materials, including their high surface area-to-volume ratio, tunable physicochemical properties, and capacity for functionalization with diverse targeting ligands.

A prime example of nanotechnology's diagnostic potential is its application in cancer detection through the identification of circulating tumor cells (CTCs). CTCs are rare cancer cells that detach from primary tumors, enter the bloodstream, and contribute to metastasis, which is responsible for approximately 90% of cancer-related deaths. These cells serve as vital biomarkers for early cancer detection, prognosis, and monitoring treatment efficacy. However, their low abundance and heterogeneity present significant challenges for conventional detection methods.

Nanotechnology has the potential to address these issues by enabling highly sensitive and specific CTC capture and analysis. For instance, nanoparticles functionalized with antibodies, aptamers, or peptides target epithelial cell adhesion molecule (EpCAM) and other surface markers uniquely expressed on CTCs can be used to discriminate CTCs from abundant circulating monocyte populations. Despite these advancements, depending on this ligand-receptor based recognition scheme means knowledge of the specific molecular make up of potential CTCs is needed prior to their detection, thereby undermining their utility as early warning signs. As a result, there is a need to develop a nanotechnology capable to detecting CTCs without direct functionalization in a tumor-agnostic fashion.

Additionally, the accurate detection and imaging of solid tumors remain critical challenges in oncology. Conventional imaging techniques, such as computed tomography (CT), magnetic resonance imaging (MRI), and positron emission tomography (PET), often struggle to differentiate malignant from benign lesions at early stages or within heterogeneous tumor environments. Moreover, current contrast agents are limited in their ability to selectively target tumor tissues, often resulting in nonspecific accumulation, suboptimal imaging contrast, and rapid half-life. These limitations underscore the need for advanced imaging technologies that enable precise tumor localization and characterization.

Nanotechnology offers transformative solutions to these challenges through the development of next-generation contrast agents. By exploiting the unique properties of nanoparticles—such as high surface area-to-volume ratio and tunable physicochemical characteristics—nanotechnology has the potential to improve the design of contrast agents by improving tumor specificity, enhanced imaging signals, minimized off-target effects, and improving the imaging time window (i.e. longer half-life).

Cationic polymers and lipid nanoparticles (LNPs) are dominant modalities; however, they have drawbacks. The vast majority of an injected LNP dose becomes trapped in the liver and is not efficiently taken up by cancerous cells after systemic injection. Cationic polymers on the other hand show strong nonspecific binding to many cells types and interact with serum components. This results in short circulation time and toxic effects. Surface shielding by PEGylation can improve stability, reduce nonspecific binding and clearance, and prolong circulation, but it also impairs cellular uptake by target cells. Gold nanoparticles (AuNPs), quantum dots (QDs), and upconversion nanoparticles (UCNPs) also have promise. These materials can be engineered to exhibit fluorescence, magnetic, or radiopaque properties, providing multimodal imaging capabilities that integrate optical, MRI, and PET imaging for comprehensive tumor visualization but they depend on peptide-functionalized to accumulate in tumor tissues even modestly.

### SUMMARY

The present disclosure presents an innovative nanotechnology-based contrast agent for solid tumor imaging. In some embodiments, the agent combines environmentally responsive targeting capabilities with multimodal imaging properties, enabling precise detection of tumor margins, intratumoral heterogeneity, and metastases. Designed for compatibility with existing imaging modalities, this platform represents a significant advancement in the detection and monitoring of solid tumors, with implications for improving patient outcomes through early and accurate diagnosis.

Disclosed herein is a self-agglomerating nanohydrogel (also referred to as “SANG”) and methods of delivering an imaging agent and detecting cancer with the nanohydrogel. Aberrant vasculature in the tumor microenvironment (TME) was demonstrated to cause SANG particles to persist longer and experience more collisions with each other in the tortuous bloodstream. Remarkably, this causes individual SANG particles to aggregate into masses that are too large to escape the tumor. This so called “self-agglomerating” characteristic emerges from the unique topology and physiochemical properties of the SANG platform and ultimately results in much higher concentrations of SANGS in tumors with aberrant vasculature. This unexpected emergent property of the SANGS platform is distinct from the classical EPR (enhanced permeability and retention) effect and results in a large concentration gradient in the TME, promoting the penetration of blood vessels by SANG particles to reach cancerous cells. Because aberrant vasculature is a highly conserved feature among solid tumors cancers, SANGS offers a realistic means to target cancers of multiple tissue origins and molecular profiles with a single platform. This innovation allows targeting of both primary and metastatic loci and will be a potential paradigm shift in cancer diagnosis and treatment.

When administered intravenously, SANGs distribute broadly but undergo concentration- and environment-dependent self-agglomeration in regions of slowed or tortuous tumor microvasculature, producing preferential accumulation and long retention in primary and metastatic solid tumors without the need for attached molecular targeting ligands. Notably, SANGs flexible surface chemistry enables them to bear (multiple) detectable moieties (e.g. fluorophores, radiolabels, MRI agents, ultrasound enhancers).

As demonstrated herein, following a systemic (e.g., IV) administration, SANGs bearing detectable moieties preferentially label tumors. Tumor cells that shed into the circulation (CTCs) can then be detected in whole blood samples using any one of the orthogonal detection modalities (fluorescence, flow cytometry, immunofluorescence imaging, ddPCR after sorting, etc.), enabling a minimally invasive screen for the presence of solid tumors. This diagnostic approach leverages SANGs as an alternative labeling modality to current ex vivo antibody-based capture/detection methods.

In addition, systemically administered SANGs (labeled with imaging compatible moiety) act as pan-tumor agent for in vivo (clinical) imaging for whole body detection of cancer and to enhance surgical debulking as a contrast agent.

Accordingly, the present disclosure provides compositions, diagnostic methods, imaging methods, kits, and specific embodiments for using SANGs as a systemic tumor-labeling and contrast agent platform, and for detecting CTCs or tumor presence in bodily fluids after systemic administration.

In certain aspects, the disclosure relates to a method of detecting or imaging a solid tumor in a subject, the method comprising:
administering to the subject a composition comprising a population of nanohydrogels associated with an imaging agent;
   wherein each of the population of nanohydrogels comprises a cross-linkable core/shell polymer;
      wherein the nanohydrogel is configured to self-agglomerate in aberrant vasculature environments,
   wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration; and
detecting the composition, thereby detecting or imaging the solid tumor.

In certain aspects, the disclosure relates to a method of detecting a solid tumor in a subject, the method comprising:
administering to the subject a composition comprising a population of nanohydrogels associated with an imaging agent;
   wherein each of the population of nanohydrogels comprises a cross-linkable core/shell polymer;
      wherein the nanohydrogel is configured to self-agglomerate in aberrant vasculature environments,
   wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration;
after an incubation period, obtaining a biological sample from the subject; and
measuring a fluorescence signal of the biological sample ex vivo released by the imaging agent, wherein
   the presence of a significant fluorescence signal indicates the presence of a solid tumor in the subject.

In certain embodiments, the imaging agent is tethered to a core of the nanohydrogel.

In certain embodiments, detecting comprises microscopy, barcode detection, scintillation counting, silicon detecting, or flow cytometry.

In certain embodiments, the imaging agent comprises a fluorophore, a near-infrared dye, a heavy metal, a radioisotope, a barcoded oligonucleotide (e.g., a barcoded DNA), or a quantum dot.

In certain embodiments, the solid tumor is ovarian cancer, colon cancer, lung cancer, breast cancer, or pancreatic cancer, such as ovarian cancer.

In certain aspects, the disclosure relates to a method of imaging a solid tumor in a subject, comprising:
administering to the subject a composition comprising a population of nanohydrogels associated with an imaging agent;
   wherein each of the population of nanohydrogels comprises a cross-linkable core/shell polymer;
      wherein the nanohydrogel is configured to self-agglomerate in aberrant vasculature environments,
   wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration, and
detecting the composition in the subject, thereby imaging the solid tumor.

In certain embodiments, the imaging agent is tethered to a core of the nanohydrogel.

In certain embodiments, the imaging agent comprises a contrast agent, a heavy metal, or a radioisotope.

In certain embodiments, the imaging agent comprises a fluorophore, a chelator for radiometals, a paramagnetic moiety, a gold nanoparticle, a SANG-fluorophore conjugate, a SANG-chelator conjugate, a SANG paramagnetic conjugate, or a SANG-gold nanoparticle conjugate.

In certain embodiments, the imaging agent comprises a contrast agent, a heavy metal, a radioisotope, a fluorophore, a chelator for radiometals, a paramagnetic moiety, a gold nanoparticle, a SANG-fluorophore conjugate, a SANG-chelator conjugate, a SANG paramagnetic conjugate, or a SANG-gold nanoparticle conjugate.

In certain embodiments, detecting comprises positron emission tomography.

In certain embodiments, detecting the composition in the subject occurs while the subject is undergoing a surgical procedure. In some such embodiments, the method further comprises surgically debulking the solid tumor after detecting the composition.

In certain embodiments, the solid tumor is ovarian cancer, colon cancer, lung cancer, breast cancer, or pancreatic cancer, such as ovarian cancer.

Other features, objects, and advantages of the invention will be apparent from the detailed description and from the claims.

### BRIEF DESCRIPTION OF DRAWINGS

FIG. 1 shows a schematic diagram of the protocol for the synthesis and preparation of fluorescently labeled SANGs. PDI, polydispersity index; DLS, dynamic light scattering.

FIG. 2 shows vector design and study design of the study described in Example 2. (panel a) vectors to induce large animal ovarian cancer (OC) selected from top 10 oncogene drivers observed in human OC. (panel b) Vector design. (panel c) study design and timeline of experimental procedures and measures. (panel d) change in weight over the 6-month study course with key experimental time points (panel c) superimposed.

FIG. 3 shows selected results from the study described in Example 2. (panel a) External view from laparoscopic identification of ovaries (panel b) prior to and (panel c) during injection (n=3). (panels d-e) Axial and coronal contrast CT image of matched pig from baseline one month after OC induction indicates contrast enhancing mass lesions at the site of oncogenic lentiviral injection. Dotted white line indicates the largest axial and coronal dimensions in each sequence. Axial and coronal T1w MRI image of matched pig three months after OC induction indicates expanded contrast enhancing mass lesions at the site of oncogenic lentiviral injection (panels f-g) with and (panels h-i) without contrast.

FIG. 4 shows selected results from the study described in Example 2. (panel a) Gross identification of tumor detected ipsilateral to vector injection. Normal ovaries were noted contralateral to vector injection and were indistinguishable from ovaries from (panel b) control animals. (panel c) Low-power view (H&E) of lentiviral injected ovary indicate. (panel d) Higher-power view of insets demonstrates sheets of tumor cells with large nuclei and prominent nucleoli and multi-nucleated cells. Tumors demonstrated cellular regions showing hyperchromasia, (panel e) high mitotic index (yellow arrowheads), (panels f-g) SET (solid, pseudoendometrioid or transitional) pattern, severe nuclear atypia and significant nuclear pleomorphism (3× variation in size), with large, peculiar, and multinucleated forms.

FIG. 5 shows selected results from the study described in Example 2. (panels a-d) representative confocal microscopic image of whole blood collected immediately prior to surgical induction of ovarian cancer. Detected cells (DAPI) are devoid of OC fluorescent reporter mCherry together with timing of collection indicate their presumptive status as circulating immune cells. (panel e-h) Blood sample collected at time of CT reveals presence of mCherry positive circulating cell (presumptive CTC). (panel i-1) Blood sample collected at time of MRI reveals presence of expanded mCherry positive circulating cells in large cluster. (m-p) Ascites fluid collected at termination indicates Cherry positive cells.

FIG. 6 shows selected results from the study described in Example 2. Representative 63× confocal microscopy orthogonal projection of (panel a) merged and (panels b-d) individual channels collected from ovarian tumor section shows proliferative marker (panel c) Ki67 ubiquitously expressed in lentiviral injected ovaries. (panel d) SANG fluorescent signal co-localized with Ki67 positive cells indicating high level of tumor targeting specificity (panel e).

FIG. 7 shows selected results from the study described in Example 2. (panels a-e) Representative image from whole blood collected and processed prior to OC induction (baseline) reveals only presumptive circulating immune cells (*), which are devoid of the OC fluorescent reporter mCherry (panel d). (panels f-j) One month after OC induction but before SANGs treatment reveal the detection of mCherry positive cell (arrow) in whole blood indicates presence of CTCs emanating from OC tumor (detected by CT), their association with multiple other circulating immune cells suggests mixed cluster formation (*). (panels k-o) Three months after OC induction and 72 hours after i.v. SANG treatment reveal continued detection of mCherry positive CTCs and their association with presumptive immune cells (*). SANG fluorescent signal detected in mCherry positive CTCs and not in circulating immune cells. (p-t) Ascites fluid collected at termination reveal 5 cell mCherry positive cluster positive for SANG fluorescence.

FIG. 8 shows physicochemical property characterization of SANG and its cellular uptake. (panel a) A schematic diagram of the protocol for the synthesis and preparation of fluorescently labeled siRNA-loaded SANGs. (panel b) Physicochemical property characterization of SANGs, which includes the hydrodynamic size, polydispersion index (PDI), zeta potential, molecular weight (MW in kDa), N:P ratio, and encapsulation efficiency (E.E.). (panel c) Negative-stain TEM image of SANGs. (panel d) reproducibility of SANG production across 3 independent batches (n=30 independent measures/batch for each core and shell) showing the complete size distribution of the core and shell components prior to final cleaning and purification. (panel e) Chemical stability of siRNA loaded and unloaded SANGs at −25 °C, 4 °C and 25 °C (mean of n=3 independent batches per condition. (panel f) Cell viabilities (Hey-A8-F8) of SANGs (75 nM) differentially loaded with negative control (NC) siRNA-EGFR (siRNA) and mir-429 (miRNA), normalized to saline treated cells (n=4 independent experiments). (panel g) Cellular uptake in Hey-A8-F8 ovarian cancer cells following overnight incubation (18 h) with Texas-Red labeled (top) and unlabeled (bottom) SANGs (yellow pseudo-colored for contrast) for both low (left, 20×) and high (right, 63×) magnification views. Cells (purple) visualized with Lectin DyLight™ 649 (Vector Laboratories). (panel h) Semi-quantitative analysis of the dose-dependent mean fluorescent intensity (MFI) measured in Hey-A8-F8 ovarian cancer cells 18 h following incubation Texas-Red labeled SANGs derived from confocal images acquired under identical conditions. Data are presented as mean±sd. (*) indicates statistically significant differences between experimental groups as empirically derived from hierarchical Bayesian model.

FIG. 9 shows mechanism of internalization, endosomal escape, and delivery of RNAi payloads. (panel a) Sodium azide (NaN₃) dependent reduction (18-hour incubation) of cellular uptake as measured by mean fluorescence intensity. (panel b) Representative confocal fluorescence images (maximum intensity projections from z-stack) showing suppressed SANG internalization by clathrin-mediated endocytosis inhibitor (chlorpromazine [Cpz]) and quantification of intracellular mean fluorescent intensity of SANGs following exposure to different doses of Cpz (18-hour incubation). (panel c) Representative confocal fluorescence images (maximum intensity projections from z-stack) showing suppressed SANG internalization by macropinocytosis inhibitor (cytochalasin D [CytoD]) and quantification of intracellular mean fluorescent intensity of SANGs following exposure to different doses of CytoD. SANGs were transfected at a final concentration of 75 nM. (panel d) Maximum intensity projections from z-stack (10 μm) confocal images of Hey-A8-F8 cells 18 hours following incubation with Cy3-siRNA-loaded SANGs (Alexa Fluor 488) at both low (right, 20×) and high (left, 63×) magnifications showing intracellular distribution. (panel e) Three-dimensional rendering of confocal images dual-labeling endosomes (CellLight™ Endosomes-GFP) and siRNA (Cy3) 18 hours following Hey-A8-F8 incubation with siRNA-loaded SANGs. (panel f) Individual fluorescent channels shown for clarity. (panel f, lower left) Results of colocalization analysis performed on a pixel-by-pixel basis between endosomes and siRNA channels, where every pixel is plotted based on its intensity level. Scatterplot colors represent pixel density. (panel g) Quantitative results of colocalization analysis summarized by the Pearson's correlation coefficients (R) between endosomes and siRNA. n=24 cellular replicates. (panel h) siRNA release from SANGs quantified by colocalization coefficients (mean±sd) between SANGs loaded with Cy3-labeled siRNA at 6 and 18 hours after incubation with Hey-A8-F8 cells. Co-localization of siRNA relative to SANGs quantifies the amount of encapsulated siRNA, n=24 cellular replicates. SANGs were transfected at a final particle concentration of 75 nM particle concentration. (*) indicates statistically significant differences between experimental groups as empirically derived from hierarchical Bayesian model (stan_glm): 95% highest density intervals do not overlap between groupwise contrasts.

FIG. 10 shows preferential biodistribution in primary and metastatic tumors and comparison with LNP. (panel a) Whole-body imaging of the mice after intravenous injection of free siRNA (Cy3-labeled) or SANGs (1 mg·kg⁻¹). Tumor bioluminescence and SANGs fluorescence were imaged sequentially in ovarian cancer bearing and control mice showing distribution at 30 min and 24 hours after i.v. injection. (panel b) Whole-body imaging of ovarian cancer-bearing mice 72 hours after intravenous injection of SANGs (1 mg·kg⁻¹) or saline control (top row). Ex vivo imaging of tumors (Hey-A8-F8) and major organs immediately imaged after in vivo imaging was completed. (panel c) Quantification of SANG delivery and retention to ovarian tumors, normalized against SANG fluorescence signal in heart. (panel d) Quantification of SANG delivery and retention across specified time points across liver, kidney, spleen, heart, lungs, and GI tract all normalized against SANG fluorescence signal in heart. Intravenous delivery of free siRNA (Cy3-labeled) used as quantitative control. (panels e-f) Representative ex vivo images at 4 and 24 hours after intravenous injection of SANGs (1 mg·kg⁻¹) to mice following metastatic tumor induction (typically 18-21 days). (panel g) Quantification of SANG delivery and retention to metastatic ovarian tumors, normalized against SANG fluorescence signal in heart. Intravenous delivery of free siRNA (Cy3-labeled) used as quantitative control. (panel h) Colocalization analysis of ex vivo SANG fluorescence and tumor bioluminescence. Dotted line of identity indicates perfect colocalization with ±15% bounding conditions (light blue). Upper quadrant shows low (dark blue) and high-intensity (red) off-target SANGs. Lower quadrant shows low (dark blue) and high-intensity (orange) untargeted cancer. (panel i) schematic illustration of the flow cytometry study to compare SANG and LNP biodistribution. (panel j) Determination by flow cytometry of the efficiency of AlexaFLuor-647 labeled siRNA against EGFR to CD31⁻CD45⁻, CD31⁺CD45⁻, and CD31⁻CD45 cell populations from liver and kidneys by LNP-LP01 (n=3 biologically independent experiments) and SANGs (n=4 biologically independent experiments) relative to PBS control (n=3). Data are presented as mean±sd. (*) indicates statistically significant differences between experimental groups as empirically derived from hierarchical Bayesian model (stan_glm): 95% highest density intervals do not overlap between groupwise contrasts.

FIG. 11 shows tumor agnostic, cross species in vivo and ex vivo biodistribution. (panel a) Sequential whole-body imaging of Pirc rats after intravenous injection of free siRNA (Cy3-labeled) or SANGs (1 mg·kg⁻¹) showing the immediate (90 min), early distribution (24 hours), and retention (1 week). (panel b) Ex vivo imaging of tumors and major organs. (panel c) Quantification of the specificity of SANG delivery and retention to tumors and major organs (liver, kidney, spleen, heart, lungs), normalized against SANG fluorescence signal in heart. Top inset shows enlarged colorectal tumor biodistribution of SANGs across time. Representative color photograph of a section the internal lumen of the descending colon of a Pirc rat indicating diffuse adenocarcinomas. (panel d) Confocal microscopy of SANG distribution in advanced colorectal cancer. Z-projections of confocal images dual-labeling vessels (Anti-alpha smooth muscle Actin) and SANGs (Texas-red) tumor, liver, and kidney extracted from identical Pirc rat 72 hours after intravenous injection of SANGs (1 mg·kg⁻¹). (panel e) Ex vivo imaging of lungs from tumor-bearing and control SRG rats 48 hours after SANG dose (1 mg·kg⁻¹). (panel f) Quantification of the specificity of SANG delivery and retention to tumors normalized against SANG fluorescence signal in heart. (panel g) Representative ex vivo images showing SANG delivery relative to differential tumor load and (panel h) quantification across all lung-tumor bearing SRG rats (n=8) and control (n=3). Data are presented as mean±sd. (*) indicates statistically significant differences between experimental groups as empirically derived from hierarchical Bayesian model (stan_glm): 95% highest density intervals do not overlap between groupwise contrasts.

FIG. 12 shows in vivo tumor penetration and efficacy. (panel a) Dose-dependent down-regulation of EGFR and KRAS mRNA levels as quantified by RT-qPCR (n=3 independent experiments) in tumor bearing mice. The relative level of mRNA expression was calculated over the NC siRNA control each of which was normalized to GAPDH. (panel b) Down-regulation of EGFR and KRAS mRNA levels as quantified by RT-qPCR (n=3 independent experiments) in Pirc rats. The relative level of mRNA expression was calculated over the NC siRNA control each of which was normalized to RPS18. (panel c) representative 20×LSCM images of tumors sections (orthotopic Hey-A8-F8) from mice 48 hours after intravenous administration of either negative control siRNA loaded SANGs (top row) or siRNA against EGFR loaded SANG (bottom row). (panels d-e) Three-dimensional (z-stack) confocal microscopic images show expanded view (63×) of white dotted sections (panel c). Intracellular distribution of endosomes and siRNA 18 hours following Hey-A8-F8 incubation with SANGs loaded with Cy3-labeled siRNA. Merged images of cells nuclei (DAPI), EGFR (Alexa Fluor 488) and SANGs (Texas Red). Data are presented as mean±sd. (*) indicates statistically significant differences between experimental groups as empirically derived from hierarchical Bayesian model (stan_glm): 95% highest density intervals do not overlap between groupwise contrasts.

FIG. 13 shows tests of tumor-agnostic targeting mechanism. (panel a) Representative phase contrast transmission electron microscopy (TEM) images showing reversible SANG agglomeration when going from 52 nM to 156 nM, and back again (re-diluted). (panel b) Observed (solid lines, data points=mean±sd) and expected (dotted lines) size and number of SANGs particles over the entire concentration range studied by TEM within a standard field of view (area). (panel c) Hydrodynamic size distributions of SANGs at 52 nM and 156 nM concentrations (n=3 independent experiments). (panel d) Diffusion coefficients derived from diffusion-ordered NMR (DOSY) for 20 ms and 100 ms pulse sequences and T2 relaxation times across a 10-fold concentration range. (panel e) Sequence of representative liquid phase transmission electron microscopy (LP-TEM) images showing two instances (outlined regions) of SANG agglomeration at 52 nM (panel f) Representative high resolution (63×) maximal projection confocal microscopic image of colon tumor showing merged view of both fluorescently labeled SANG populations and DAPI. Scatter plot of pixel-based colocalization analysis of representative areas (Costes randomization based colocalization R=0.87±0.02, p<0.001, n=3 animals, 3 sections each); a value of 0.5 indicates purely random co-localization probability. (panel g) Representative high resolution (63×) maximal projection confocal microscopic image of healthy liver showing merged view of both fluorescently labeled QP VLP populations and DAPI. Scatter plot of pixel-based colocalization analysis of representative areas (Costes randomization based colocalization R=0.52±0.02, p<0.001, n=3 animals, 3 sections each). (panel h) Graphical depiction of provisional mechanism by which SANGs achieve preferential tumor by exploiting maladaptive vasculature observed uniquely in tumor microenvironments (bottom) compared to healthy vasculature (top). (*) indicates statistically significant differences between experimental groups as empirically derived from hierarchical Bayesian model (stan_glm): 95% highest density intervals do not overlap between groupwise contrasts.

FIG. 14 shows synthesis, loading, and physiochemical properties of SANGs. (panel a) A schematic diagram of the protocol for the synthesis and preparation of fluorescently labeled siRNA-loaded SANGs. PDI, polydispersity index; DLS, dynamic light scattering. (panel b)¹H nuclear magnetic resonance (NMR) spectra of SANGs in D₂O at 52, 130, and 520 nM concentration in particle at an estimated molecular mass of 1.93e⁴ kDa. (panel c) Dynamic light scattering distributions, Zeta potential and distributions of empty SANGs and after siRNA loading of negative control (nc) and active (EGFR) siRNA. (panel d) Empirically derived siRNA concentration curve for the quantification of encapsulation and loading efficiency.

FIG. 15 shows National Characterization Laboratory (NCL) Data. Physiochemical properties, stability, and contamination results (endotoxin) from independent validation at NCL.

FIG. 16 shows dose- and time-dependent internalization. (panel a) Representative merged confocal fluorescence microscopy images (63×) of Hey-A8-F8 ovarian cancer cells (white) after 18 hours of incubation as function of SANG concentration (red). Individual SANG fluorescence channel shown for clarity. (panel b) quantification of SANG mean fluorescent intensity in Hey-A8-F8 ovarian cancer cells at indicated times measured from (63×) confocal images. (panel c) Representative merged confocal fluorescence microscopy images (20×) of Hey-A8-F8 ovarian cancer cells (white) as a function of SANG incubation time (red).

FIG. 17 shows SANG internalization in a variety of cell types. representative confocal fluorescence microscopy images (63×) of (panel a) MCF7 breast cancer, (panel b) OVCAR3 ovarian cancer, and (panel c) IOSE epithelial ovarian cells following overnight incubation (18 h) with Texas-Red labeled (top) SANGs (52 nM).

FIG. 18 shows dose-dependent internalization inhibitors. Quantification of intracellular mean fluorescent intensity of SANGs following exposure to different doses of (panel a) clathrin-mediated endocytosis inhibitor (chlorpromazine [Cpz]), (panels b,c) macropinocytosis inhibitors (latrunculin A [Lat] and cytochalasin D [CytD]), and (panel d) caveolae-mediated inhibitor (methyl-o-cyclodextrin [Mpcd]). Maximum intensity projections from z-stack (63×) confocal images of Hey-A8-F8 cells 18 h following incubation with SANGs. SANGs were transfected at a final concentration of 75 nM. Data are presented as mean±sd. (*) indicates statistically significant differences between experimental groups as empirically derived from hierarchical Bayesian model.

FIG. 19 shows endosomal uptake and release of siRNA. Merged super-resolution confocal(63×) confocal images of Hey-A8-F8 cells 18 h following incubation with SANGs loaded with fluorescently labeled siRNA (right). Individual fluorescent channels (left). Endosomes labeled with wheat germ agglutinin (WGA) a lectin derivative (purple arrows). WGA was applied for 15 minutes prior to imaging. Data reveal a subpopulation of SANGs were actively internalized during WGA incubation (white arrows) while other subpopulations remained devoid of WGA signal (red arrows). * indicate siRNA distributed throughout cytoplasm.

FIG. 20 shows SANGs escape endosomes. Three-dimensional rendering of confocal images dual-labeling endosomes (CellLight™ Endosomes-GFP) and SANGs (Texas-red) in Hey-A8-F8 labeled with membrane (phalloidin, purple) and nuclear counter (Hoechst, blue) stains. Two independent biologic experiments shown in (panels a-c) and (panels d-f). Individual channels shown for clarity in c1-5 and f1-5. (panel g) siRNA release from SANGs quantified by colocalization coefficients (mean±sd) between SANGs and loaded Cy3-labeled siRNA at 6 and 18 hours after incubation with Hey-A8-F8 cells. n=24 cellular replicates. Representative three-dimensional renderings of confocal images shown for (panel h) 6 hour and (panel i) 18 hour time points. SANGs were transfected at a final particle concentration of 75 nM. Surface renderings of SANGs and Cy3-siRNA channels in two representative cells studied at 6 and 18 hours after incubation. (*) indicates statistically significant differences between experimental groups as empirically derived from hierarchical Bayesian model (stan_glm): 95% highest density intervals do not overlap between groupwise contrasts.

FIG. 21 shows in vitro functionality. (panel a) representative confocal fluorescence microscopy images (20×) show suppression of EGFR protein expression levels in Hey-A8-F8 cells 48 hours after siRNA against EGFR loaded SANG (top row) as compared to negative control siRNA loaded SANGs (bottom row). n=3 replicates. In both instances, cells were incubated with SANGs for 12 hours after which media was replaced for remainder of the study. (panel b) Down-regulation of EGFR, KRAS, ZEB1, and Glut1 mRNA levels as quantified by RT-qPCR. The relative level of mRNA expression was calculated over the NC siRNA control. n=3 replicates. (panel c) phenotypic shift of canonically mesenchymal Hey-A8-F8 ovarian cancer cells to epithelia phenotype following 24 hours incubation with SANGs loaded with mir-429. Scalebar, 10M. Data are presented as mean±sd. (*) indicates statistically significant differences between experimental groups as empirically derived from hierarchical Bayesian model (stan_glm): 95% highest density intervals do not overlap between groupwise contrasts.

FIG. 22 shows SANG Biodistribution in breast cancer. (panel a) Whole-body imaging of mice 24 h after intravenous injection of SANGs (1 mg·kg⁻¹). Tumor bioluminescence and SANGs fluorescence were imaged sequentially in breast cancer bearing mice (orthotopically implanted MDA-MD-231). (panel b) Whole-body imaging of cancer-bearing mice 72 hours after intravenous injection of SANGs (1 mg·kg⁻¹). (panel c) Ex vivo imaging of tumors (MDA-MB-231) and major organs immediately imaged after in vivo imaging was completed.

FIG. 23 shows SANG biodistribution and siRNA delivery do not depend on surface functionalization. Whole-body imaging of mice 72 h after intravenous injection of saline, SANGs (1 mg·kg⁻¹), or nanohydrogels (1 mg·kg⁻¹) functionalized with the 12 peptide ephrin-A1 ligand mimic (YSAYPDSVPMMS) SANG. YSA peptide is designed to bind erythropoietin-producing hepatocellular (Eph) A2 receptor ubiquitously expressed on ovarian cancer cells.

FIG. 24 shows raw biodistribution. Quantification of ex vivo imaging of tumors and major organs immediately imaged after in vivo imaging was completed in relation to FIG. 10 for rat colorectal cancer (panel a) and for mouse ovarian cancer (panel b).

FIG. 25 shows epidural metastasis spinal cord (panel a) Ex vivo imaging of the brain and spinal cord from an ovarian cancer (Hey-A8-F8) bearing mouse 72 h after intravenous injection of SANGs (1 mg·kg⁻¹). Tumor bioluminescence and SANGs fluorescence were imaged sequentially.

FIG. 26 shows Met targeting quantification. (panel a) Representative ex vivo images of tumor bioluminescence and SANGs fluorescence from ovarian cancer (Hey-A8-F8) bearing mouse following metastatic tumor induction (typically 18-21 days) 4 h after intravenous injection of SANGs (1 mg·kg⁻¹) as shown in FIG. 10. (panel b) Image masks utilized for colocalization analysis. (panel c) Computed distributions of colocalization among BLI and SANG channels following 1000 iterations of Costes randomization (blue) and observed colocalization (red line) with one representative shuffled image shown (Costes randomization based colocalization R=0.85±0.02, p<2.2⁻¹⁶, n=3 animals, 3 sections each) (panel d) Quantification of ex vivo imaging of tumors and major organs immediately imaged after in vivo imaging was completed in relation to FIG. 10 for rat colorectal cancer (a) and for mouse ovarian cancer (b).

FIG. 27 shows comparative analysis of SANGs and LNP. (panel a) Ex vivo imaging of excised organs from ovarian cancer (Hey-A8-F8) bearing mice 1 h and 4 h after intravenous injection of (panel b) LNP-LP01 formulated to carry AlexaFluor-647 labeled siRNA against EGFR and (panel c) SANGs formulated to carry Texas-Red labeled siRNA against EGFR. (panel d) Flow cytometry uptake studies of LNP-LP01 formulated to carry AlexaFluor-647 labeled siRNA against EGFR (n=3 biologically independent experiments) compared to SANGs formulated to carry AlexaFluor-647 labeled siRNA against EGFR (n=4 biologically independent experiments) and PBS control (n=3) in CD31⁻CD45⁻, CD31⁺CD45⁻, and CD31⁻CD45⁺ cell populations isolated from liver and kidneys. Cell populations were first gated for non-debris (forward scatter versus side scatter), then gated for singlets (forward scatter versus forward scatter width) and viable cells (Zombie). The viable, single cells were analyzed for AlexaFluor-647 positivity to determine the percentage of AlexaFluor-647 positive cells in each sample. The AlexaFluor-647 median fluorescence intensity was normalized to the autofluorescence of each tissue type, as determined using samples from PBS injected mice.

FIG. 28 shows time-dependent internalization in rats with advanced colorectal cancer. Whole-body imaging of Pirc rats after intravenous injection of free siRNA or SANGs (1 mg·kg⁻¹). SANGs fluorescence were imaged sequentially at indicated times in FIG. 11 in cancer bearing animals.

FIG. 29 shows pharmacokinetics and elimination. Empirical quantification of SANG concentration in blood (blood) along with bi-exponential model fit (red) after single bolus injections in SD rats. Dotted (purple) and solid (yellow) lines shows siRNA concentration and background fluorescence over time. Inset shows urine and feces elimination of naked siRNA and SANG dose during the first 24 h. Data are presented as mean±sd (n=3 rats per group).

FIG. 30 shows tumor penetration. (panel a) Whole-body in vivo imaging of representative Pirc rat after 1 week after intravenous injection of SANGs (1 mg·kg⁻¹) with corresponding confocal microscopic images of EGFR overexpressing colorectal cancer cells and SANGs near-infrared fluorescence demonstrating tumor penetration and retention behavior. (b top) Representative merged confocal fluorescence microscopy image (20×) and (panel b bottom) expanded (63× of dotted white box) of advanced colorectal tumors, 72 hours after i.v. injection of SANGs (red). (panel c) Whole-body in vivo bioluminescence imaging OC tumor-bearing mice assessing tumor load prior to and 6 days after cohorts of animals were treated with saline (n=5), cisplatin (n=4), or combined miRNA-429 and siRNA against EGFR loaded SANGs (n=5). (panel d) Quantification of wet tumor weights following last in vivo imaging.

FIG. 31 shows SANGs are minimally toxic in mouse. (panel a) Serum levels of ALP, BUN and IL6 at the completion of the therapy study in Hey-A8-F8 ovarian tumor-bearing mice. (panel b) Mouse weights during the study in panel (a), measured once every 2 days. Data are presented as mean±sd (n=5 mice per group). (panel c) Percentage change of starting weight in response to two i.v. 7 mg·kg⁻¹ doses of either (1) empty NG or (2) NG loaded with siRNA or miRNA administered to female CD-1 mice (5 animals/group) separated by 24 hours. The error bands represent ±95% CI. (panel d) Complete blood chemistry metrics 14 days after the final dose in CD-1 mice. Data are presented as mean±sd unless otherwise noted. (*) indicates statistically significant differences between experimental groups as empirically derived from hierarchical Bayesian model (stan_glm): 95% highest density intervals do not overlap between groupwise contrasts.

FIG. 32 shows SANGs are minimally toxic in rat and porcine models. (panel a) Complete blood chemistry metrics at 6 and 24 hours after i.v. administered of SANGs loaded with siRNA against EGFR at 0, 7, 12, 17 mg·kg⁻¹ in Sprague-Dawley rats. (panel b) Representative sections of key organ systems (from animals terminated at 24 hrs) evaluated by independent-blinded clinical pathologist. No differences detected between saline injected animals and SANG-EGFR treated animals>96× starting human doses. Shown are representative images from 4 independent experiments. (panel c) Rats with advanced colorectal cancer (Pirc) received a modeled anticipated clinical treatment schedule. SANG-siEGFR (7 mg·kg⁻¹) was infused twice weekly for 5 weeks along with a co-infusion of oxaliplatin (5 mg·kg⁻¹). Weights were measured twice weekly as a surrogate measure for tolerability. Data are presented as mean±sd (n=5 mice per group). (panel d) time series of complete blood chemistry from single-acute dose study in an adult female (73 kg) Yucatan swine showed no clinically meaningful deviances out to 6 hours following i.v. infusion of SANGs-siRNA at the same high dose (7 mg·kg⁻¹) with the exception of a transient increase in AST at 5 minutes (*).

FIG. 33 shows concentration dependent size change in SANGs as measured by TEM and DLS. (panel a) Fixed view negative-stain TEM images as a function of the SANG concentration and (panel b) corresponding particle analysis masks. (panel c) Hydrodynamic sizes of SANGs at sequentially escalating sample concentrations followed by re-dilution (grey inset). Dynamic light scattering peak data are presented as mean±sd (n=3).

FIG. 34 shows DOSY raw data. Attenuation of the NMR signals during pulsed field gradient experiments. Diffusion coefficients derived from best fit curves. Measured T1-T2 correlations as a function of the SANG concentration, obtained by DOSY with D₂O and SANG distributions labeled.

FIG. 35 shows dual-color full comparison. (panel a) Representative 20×LSCM tiled image of colon with diffuse adenocarcinomas characterized by marked dysplasia in crypts and villus (box 1) and that penetrate the smooth muscle layers (box 2) showing merged view of both fluorescently labeled SANG populations and DAPI. (panels b-c) High resolution (63×) maximal projection confocal microscopic images show expanded view of white dotted sections (panel e). Scatter plot of pixel-based colocalization analysis of two representative areas (Costes randomization based colocalization R=0.85±0.02, p<0.001, n=3 animals, 3 sections each). (panel d) Size distribution of colocalized or not-colocalized SANGs derived from object-based analysis of 63× views (n=3 animals, 3 sections). (panel e) Representative three-dimensional rendering of confocal images of livers dual-labeled with QP-VLP 1 h following intravenous delivery to mice (n=3 animals, 3 sections).

FIG. 36 shows comparative proteomics profiling of individual nanoparticle platform coronas. (panel a) schematic illustration of the multi-platform comparative protein corona study. Plasma freshly isolated from healthy Wistar rats were aliquoted and incubated with either LNP or SANG nanoparticles for 3 hours (n=5 replicates per group). A control plasma sample was included to measure background plasma contamination retained through the wash steps. After incubation, ultracentrifugation washing (3×) removed free and non-specifically bound biomolecules. After each wash, the pellet was resuspended in buffer. After the final wash, proteins were digested in preparation for untargeted proteomics analysis by LC-MS. (panel b) density heatmap shows scaled total protein abundances detected on protein corona of each nanoparticle platform (n=5, biologic replicates) after ultracentrifugation washing. Plasma samples (n=5, biologic replicates) included to show residual uncomplexed plasma proteins not removed by identical ultracentrifugation (3×) washing of nanoparticle platforms and provides background protein level control for comparison. Mean (solid), 25%, 50%, and 75% (dotted) lines indicate quantiles. (panel c) Venn diagram shows the number (%) of shared and unique corona proteins associated with different nanoparticle platforms after filtering for FDR<0.001 and >1.5-fold-change compared to plasma proteins. Unique and shared proteins described in Table 1. (panel d) hierarchical clustering of nanoparticle platforms. Vertical clustering displays similarities between samples, while horizontal clusters reveal the relative abundances and groups of proteins that distinguish nanoparticle platforms. Red and blue indicate higher and lower than the mean protein signal, respectively. Scale bars represent row Z-scores. Color coded top molecular function (MF), cellular component (CC), and biologic process (BP) associated with each protein. Proteins and abundances in each numbered cluster (1-8) detailed in Table 2.

FIG. 37 shows flow cytometry quantification of SANG intracellular intensity. Determination of SANG detection threshold following in vitro exposure of cancer cells at 4- and 18-hour incubation. Percentage of cancer cells (GFP+) that co-expressed SANGs fluorescence in a time and concentration dependent manner to optimize the in vitro performance.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

### DEFINITIONS

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to a “polymer” includes aspects having two or more such polymers unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

Appropriate methods of administering a composition (e.g., a composition comprising SANGs, a composition comprising SANGs bearing detectable moieties, a composition comprising SANGs labeled with imaging compatible moiety) to a subject will also depend, for example, on the age and/or the physical condition of the subject and the chemical and biological properties of the composition (e.g., solubility, digestibility, bioavailability, stability and toxicity). In some embodiments, a composition is administered orally, e.g., to a subject by ingestion. In some embodiments, the orally administered composition is in an extended release or slow release formulation, or administered using a device for such slow or extended release.

The term “biocompatible” generally refers to a material and any metabolites or degradation products thereof that are generally non-toxic to the recipient and do not cause significant adverse effects to the subject.

The term “cancer” refers to a malignant tumor (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “control” is an alternative subject or sample used in an experiment for comparison purposes. A control can be “positive” or “negative.”

As used herein, the term “crosslinkable” in the context of crosslinkable macromolecules or polymers refers to the ability of macromolecules or polymers to form at least one or more covalent bonds (crosslinks) with one another to form a polymeric network (nanohydrogel) under a conditions effective.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ).

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

The term “selectivity” in the context of therapeutic delivery refers to the relative portion of an administered dose that reaches the targeted location. Thus, the term “tumor selectively” or the like refers to the amount of the administered dose of agent localized within a particular tumor microenvironment.

The terms “treat,” “treating,” “treatment,” and grammatical variations thereof as used herein, include partially or completely delaying, alleviating, mitigating or reducing the intensity of one or more attendant symptoms of cancer or condition and/or alleviating, mitigating or impeding one or more symptoms of cancer. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), after an established development of cancer, or during prevention or mitigation of cancer relapse. Prophylactic administration can occur for several minutes to months prior to the manifestation of a disease.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “tumor” refers to an abnormal mass of tissue wherein the growth of the mass surpasses and is not coordinated with the growth of a normal tissue. A tumor may be “benign” or “malignant,” depending on the following characteristics: degree of cellular differentiation (including morphology and functionality), rate of growth, local invasion, and metastasis. A “benign tumor” is generally well differentiated, has characteristically slower growth than a malignant tumor, and remains localized to the site of origin. In addition, a benign tumor does not have the capacity to infiltrate, invade, or metastasize to distant sites. Exemplary benign tumors include, but are not limited to, lipoma, chondroma, adenomas, acrochordon, senile angiomas, seborrheic keratoses, lentigos, and sebaceous hyperplasias. In some cases, certain “benign” tumors may later give rise to malignant tumor, which may result from additional genetic changes in a subpopulation of the tumor's neoplastic cells, and these tumors are referred to as “pre-malignant tumor.” An exemplary pre-malignant tumor is a teratoma. In contrast, a “malignant tumor” is generally poorly differentiated (anaplasia) and has characteristically rapid growth accompanied by progressive infiltration, invasion, and destruction of the surrounding tissue. Furthermore, a malignant tumor generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue. Reference is also made to the term “tumor microenvironment,” which refers to any and all elements of the tumor milieu including elements that create a structural and or functional environment for the malignant process to survive and/or expand and/or spread.

### Methods of Detection

In some aspects, described herein is a method of detecting a solid tumor in a subject, the method comprising:
administering to the subject a composition comprising a population of nanohydrogels associated with an imaging agent;
   wherein each of the population of nanohydrogels comprises a cross-linkable core/shell polymer;
      wherein the nanohyrogel is configured to self-agglomerate in aberrant vasculature environments,
   wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration;
after an incubation period, obtaining a biological sample from the subject; and
measuring a fluorescence signal of the biological sample (such as a blood, plasma, or serum sample) ex vivo released by the imaging agent, wherein the presence of a significant fluorescent signal indicates the presence of a solid tumor.

As used herein, a “significant fluorescence signal” is a fluorescence signal that is above baseline or background fluorescence, as determined by one of skill in the art. Sources of background fluorescence include, but are not limited to, background autofluorescence of biological samples.

In certain embodiments, the composition further comprises one or more pharmaceutically acceptable carriers.

Suitable nanohydrogels are described in International Application No. PCT/US2024/040745, which is incorporated herein by reference in its entirety.

In certain embodiments, the imaging agent is tethered to a core of the nanohydrogel.

In some embodiments, the detecting comprises flow cytometry, fluorescence-assisted cell sorting, microscopy, silicon detecting, or spectrophotometry. In certain embodiments, the detecting comprises microscopy.

Also described herein is a method of delivering an agent to a tumor of a subject. For example, the method can include: administering any of the compositions described herein to the subject; wherein the population of nanohydrogels is inducible to self-agglomerate to form an aggregated cluster of nanohydrogels via the aberrant microvasculature of a tumor microenvironment.

In some embodiments, the method further includes: allowing the agent to be released from the aggregated cluster of nanohydrogels. Advantageous to the disclosed platform, the self-agglomeration property permits consolidation and sustained release of the agent to the tumor microenvironment. After sufficient exposure, the agglomerated nanohydrogels begin to dissociate and extravasate to tumor interstitial spaces, leading to the selective delivery of the agent.

Further described herein are methods of detecting cancer in a subject, comprising: administering any of the compositions described herein to the subject; wherein the population of nanohydrogels is passively inducible to self-agglomerate to form an aggregated cluster of nanohydrogels via the aberrant microvasculature of a tumor microenvironment; and detecting the composition, thereby detecting the cancer. In some embodiments, the method further comprises treating the subject by administering a therapeutic agent.

In some embodiments, the present disclosure is directed to a method of treating a cancer in a subject, comprising: (a) determining the subject has cancer, by administering a composition comprising a nanohydrogel associated with an imaging agent as described herein; and (b) administering a therapeutic agent to the subject, thereby treating the cancer. Therapeutic agents suitable for use in the methods of the present disclosure include, but are not limited to, cancer therapeutic agents known in the art.

In some embodiments, the cancer is ovarian cancer, colorectal cancer, or breast cancer. In some embodiments, the cancer is ovarian cancer. In some embodiments, the cancer is colorectal cancer.

In some embodiments, the solid tumor is ovarian cancer, colon cancer, lung cancer, breast cancer, or pancreatic cancer, such as ovarian cancer.

In some embodiments, a tumor selectivity of the nanohydrogel is 1% or greater (e.g., 1.5% or greater, 2% or greater, 2.5% or greater, 3% or greater, 3.5% or greater, 4% or greater, 4.5% or greater, 5% or greater, 5.5% or greater, 6% or greater, 6.5% or greater, 7% or greater, 7.5% or greater, 8% or greater, 9.5% or greater, 10% or greater, 10.5% or greater, 11% or greater, 11.5% or greater, 12% or greater, 12.5% or greater, 13% or greater, 13.5% or greater, 14% or greater, 14.5% or greater, 15% or greater, 20% or greater, or 25% or greater). In some embodiments, the delivery efficiency of the nanohydrogel to the tumor microenvironment is measured as a ratio between the amount of nanohydrogel in the tumor relative to the amount entrained in the liver. In some aspects, the tumor:liver ratio (i.e., a ratio of relative delivery of the nanohydrogels between the tumor and a liver of the subject) is 1:1 or more (e.g., 1.5:1 or more, 2:1 or more, 2.5:1 or more, 3:1 or more, 3.5:1 or more, 4:1 or more, 4.5:1 or more, 5:1 or more, 5.5:1 or more, 6:1 or more, 6.5:1 or more, 7:1 or more, 7.5:1 or more, 8:1 or more, 8.5:1 or more, 9:1 or more, 9.5:1 or more, 10:1 or more, 10.5:1 or more, 15:1 or more, 20:1 or more, or 25:1 or more). In some embodiments, 1% or more of the population of nanohydrogels is retained after 24 hours from the initial dose (e.g., 1.5% or more, 2% or more, 2.5% or more, 3% or more, 3.5% or more, 4% or more, 4.5% or more, 5% or more, 5.5% or more, 6% or more, 10% or more). In some embodiments, 2.5% or more of the population of nanohydrogels is retained after 24 hours from an initial dose (e.g., 3% or more, 3.5% or more, 4% or more, 4.5% or more, 5% or more, 5.5% or more, 6% or more, 10% or more).

In vivo application of the disclosed nanohydrogels and compositions containing them, can be accomplished by any suitable method and technique presently or prospectively known to those skilled in the art. For example, the disclosed nanohydrogels can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. In certain aspects, the administration can include intravenous, intrathecal, intracranial, also intramuscular, intratumoral, intratracheal, subcutaneous application. Administration of the disclosed nanohydrogels or compositions can be a single administration, or at continuous or distinct intervals as can be readily determined by a person skilled in the art.

### Methods of Imaging

In certain aspects, the disclosure relates to a method of imaging a solid tumor in a subject, comprising:
administering to the subject a composition comprising a population of nanohydrogels associated with an imaging agent;
   wherein each of the population of nanohydrogels comprises a cross-linkable core/shell polymer;
      wherein the nanohydrogel is configured to self-agglomerate in aberrant vasculature environments,
   wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration, and
detecting the composition in the subject, thereby imaging the solid tumor.

In certain embodiments, the composition further comprises one or more pharmaceutically acceptable carriers.

Suitable nanohydrogels are described in International Application No. PCT/US2024/040745, which is incorporated herein by reference in its entirety.

In certain embodiments, the imaging agent is tethered to a core of the nanohydrogel.

In some embodiments, the detecting comprises positron emission tomography, such as whole body positron emission tomography. In other embodiments, the detecting comprises magnetic resonance imaging or computed tomography.

Also described herein is a method of delivering an imaging agent to a tumor of a subject. For example, the method can include: administering any of the compositions described herein to the subject; wherein the population of nanohydrogels is inducible to self-agglomerate to form an aggregated cluster of nanohydrogels via the aberrant microvasculature of a tumor microenvironment.

In some embodiments, the method further includes: allowing the agent to be released from the aggregated cluster of nanohydrogels. Advantageous to the disclosed platform, the self-agglomeration property permits consolidation and sustained release of the agent to the tumor microenvironment. After sufficient exposure, the agglomerated nanohydrogels begin to dissociate and extravasate to tumor interstitial spaces, leading to the selective delivery of the agent.

Further described herein are methods of detecting cancer in a subject, comprising: administering any of the compositions described herein to the subject; wherein the population of nanohydrogels is passively inducible to self-agglomerate to form an aggregated cluster of nanohydrogels via the aberrant microvasculature of a tumor microenvironment; and detecting the composition, thereby detecting the cancer. In some embodiments, the method further comprises treating the subject by administering a therapeutic agent.

In some embodiments, the present disclosure is directed to a method of treating a cancer in a subject, comprising: (a) determining the subject has cancer, by administering a composition comprising a nanohydrogel associated with an imaging agent as described herein; and (b) administering a therapeutic agent to the subject, thereby treating the cancer. Therapeutic agents suitable for use in the methods of the present disclosure include, but are not limited to, cancer therapeutic agents known in the art.

In some embodiments, the solid tumor is ovarian cancer, colon cancer, lung cancer, breast cancer, or pancreatic cancer.

In some embodiments, a tumor selectivity of the nanohydrogel is 1% or greater (e.g., 1.5% or greater, 2% or greater, 2.5% or greater, 3% or greater, 3.5% or greater, 4% or greater, 4.5% or greater, 5% or greater, 5.5% or greater, 6% or greater, 6.5% or greater, 7% or greater, 7.5% or greater, 8% or greater, 9.5% or greater, 10% or greater, 10.5% or greater, 11% or greater, 11.5% or greater, 12% or greater, 12.5% or greater, 13% or greater, 13.5% or greater, 14% or greater, 14.5% or greater, 15% or greater, 20% or greater, or 25% or greater). In some embodiments, the delivery efficiency of the nanohydrogel to the tumor microenvironment is measured as a ratio between the amount of nanohydrogel in the tumor relative to the amount entrained in the liver. In some aspects, the tumor:liver ratio (i.e., a ratio of relative delivery of the nanohydrogels between the tumor and a liver of the subject) is 1:1 or more (e.g., 1.5:1 or more, 2:1 or more, 2.5:1 or more, 3:1 or more, 3.5:1 or more, 4:1 or more, 4.5:1 or more, 5:1 or more, 5.5:1 or more, 6:1 or more, 6.5:1 or more, 7:1 or more, 7.5:1 or more, 8:1 or more, 8.5:1 or more, 9:1 or more, 9.5:1 or more, 10:1 or more, 10.5:1 or more, 15:1 or more, 20:1 or more, or 25:1 or more). In some embodiments, 1% or more of the population of nanohydrogels is retained after 24 hours from the initial dose (e.g., 1.5% or more, 2% or more, 2.5% or more, 3% or more, 3.5% or more, 4% or more, 4.5% or more, 5% or more, 5.5% or more, 6% or more, 10% or more). In some embodiments, 2.5% or more of the population of nanohydrogels is retained after 24 hours from an initial dose (e.g., 3% or more, 3.5% or more, 4% or more, 4.5% or more, 5% or more, 5.5% or more, 6% or more, 10% or more).

In vivo application of the disclosed nanohydrogels and compositions containing them, can be accomplished by any suitable method and technique presently or prospectively known to those skilled in the art. For example, the disclosed nanohydrogels can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. In certain aspects, the administration can include intravenous, intrathecal, intracranial, also intramuscular, intratumoral, intratracheal, subcutaneous application. Administration of the disclosed nanohydrogels or compositions can be a single administration, or at continuous or distinct intervals as can be readily determined by a person skilled in the art.

In certain embodiments, the disclosure relates to in vivo tumor labeling and imaging. In certain such embodiments, in vivo tumor labeling and imaging comprises the following steps:1) systemically administering SANGs (e.g., intravenous infusion) to the subject. The SANGs may bear a detectable label compatible with the intended imaging modality (optical, PET, MRI, ultrasound); 2) allowing circulation/accumulation time (examples: biodistribution and tumor signal progressively increase over 1 week). Image at appropriate time points post-administration to detect tumor-specific signal; and 3) imaging using the chosen modality.

Without wishing to be bound by any particular theory, applications of the methods described herein may include: a) non-invasive whole-body imaging to locate primary and metastatic solid tumor foci; b) surgical guidance where intraoperative fluorescence imaging is used (SANG fluorophores); and c) as a companion diagnostic to detect tumor burden or monitor therapy response via repeat SANG imaging.

In certain embodiments, the disclosure relates to systemic labeling and ex vivo CTC detection (diagnostic workflow)

In certain such embodiments, one diagnostic approach enabled by SANGs uses systemic labeling followed by ex vivo detection of labeled tumor cells (CTCs) in blood and comprises the following steps: 1) systemic administration of labeled SANGs (as described in In vivo tumor labeling and imaging); 2) wait a clinically determined interval sufficient for (a) systemic distribution; (b) SANGs to preferentially agglomerate and label tumor vasculature and tumor cells; and (c) labeled tumor cells to enter circulation (example time windows: 4-72 h based on preclinical biodistribution; exact clinical timing to be optimized); 3) obtain a peripheral blood sample (whole blood); and 4. Detect SANG-labeled cells using flow cytometry (direct detection of fluorophore), imaging cytometry, immunofluorescence microscopy, microfluidic capture followed by imaging, or molecular detection if barcoding or nucleic acid payloads were included. Because SANG labeling is internalized and retained by tumor cells, detection specificity and signal stability are enhanced.

In certain embodiments, the above diagnostic approach may overcome limitations of ex vivo antibody-capture methods that depend on surface antigen expression and are prone to false negatives due to epithelial-to-mesenchymal transitions. In certain embodiments, the systemic labeling step of the above diagnostic approach converts an in vivo tumor characteristic (SANG accumulation/retention) into a robust ex-vivo detection signal in blood. In certain embodiments, the above diagnostic approach is broadly applicable across tumor types because the SANG tumor selectivity is derived from tumor vasculature geometry and the SANG agglomeration mechanism rather than tumor-specific antigens.

In certain embodiments, the disclosure relates to in vitro labeling CTC detection (diagnostic workflow). In certain such embodiments, one diagnostic approach enabled by SANGs uses in vitro labeling followed by detection of labeled tumor cells (CTCs) in blood and comprises the following steps: 1) Obtain a peripheral blood sample (whole blood); 2) SANGs are used to label cancer cells in vitro following optimal incubation times and concentrations; and 3) Detect SANG-labeled cells using flow cytometry (direct detection of fluorophore), imaging cytometry, immunofluorescence microscopy, microfluidic capture followed by imaging, or molecular detection if barcoding or nucleic acid payloads were included.

In certain embodiments, the disclosure relates to hybrid imaging/diagnostic paradigms that: 1) combines in vivo imaging (to localize/take a diagnostic baseline) and ex vivo CTC detection (to increase sensitivity and enable molecular assays); and 2) uses dual-labeled SANGs (orthogonal fluorophores or imaging tag+nucleic acid barcode) to allow both imaging and robust downstream molecular analyses.

### Self-Agglomerating Nanohydrogels (SANGs)

SANGs are core-shell polymeric nanohydrogels composed of polyacrylamide-derived monomers prepared by a two-stage precipitation polymerization process. The shell may contain a small fraction of amine-bearing monomer (e.g., APMA ~0.5% of total monomer during shell formation) to allow for covalent labeling with NHS-ester fluorophores or other conjugation chemistries. Typical hydrodynamic diameters are in the −100 nm range (e.g., −110 nm) with narrow PDI, and zeta potentials near neutral to slightly negative.

In some embodiments, the crosslinkable core/shell polymer comprises a gel-forming polymer. In some embodiments, the gel-forming polymer comprises a biocompatible polymer.

Various gel-forming polymers are known in the art and include both natural and synthetic materials or combinations and copolymers thereof. In some aspects, the gel-forming polymer comprises a synthetic material. Examples of synthetic gel-forming polymers include, but are not limited to, polyacrylamide, polylactic acid (PLA), poly(lactic-co-glycolic acid (PLGA), polyethylene glycol (PEG), polyethylene glycol-co-propylene glycol (PEO-PPO), and polyacrylates. In some aspects, the gel-forming polymer comprises a natural gel-forming material. Examples of natural gel-forming polymers include but are not limited to polysaccharides, such as starch, alginate, agarose, cellulosic derivatives, such as, for example, but not limited to, hydroxypropyl methyl cellulose (HPMC), carboxymethyl cellulose (CMC) and n-ethyl cellulose, hyaluronic acid, chitosan, xanthan gum, dextran, alginate, and agar.

In some embodiments, the crosslinkable core/shell polymer comprises polyacrylamide. In some aspects, the crosslinkable core/shell polymer comprises a copolymer of N-isopropylmethacrylamide and N,N′-methylenebis(acrylamide). In some embodiments, a shell of the nanohydrogel comprises a cationic polymer ligand (e.g., poly(aminopropyl methacrylate)). In some embodiments, the shell of the nanohydrogel comprises an amine-doped polymer. In some embodiments, the shell of the nanohydrogel comprises aminomethyl propionic acid (AMPA).

Methods of synthesizing nanohydrogels are generally known in the art. As an example, core/shell nanohydrogels can be synthesized via precipitation polymerization, such as that described by Lyon et al. (2009) [10.1021/bc800547c; 10.1007/s00396-007-1805-7], which is incorporated by reference in its entirety. Briefly, precipitation polymerization involves the use of solvents or solvent mixtures in which the monomers to be polymerized are soluble, but not the polymer which is formed. In some aspects, the nanohydrogels are formed using free-radical precipitation in which monomers are dissolved/dispersed in a polar solvent or solvent mixtures before initiating the polymerization using a free radical-forming compound.

In some aspects, the crosslinkable core/shell polymer is unfunctionalized. Prior published uses of hydrogels invariably included the attachment of molecules (termed “targeting ligands” here) known to bind to cell surface markers characteristic of cancer cells or other target cells. It was shown that SANGs lacking any targeting ligand (designated as “unfunctionalized SANGs”) were able to effectively and selectively distribute to tumor tissue upon intravenous administration and to operate as a therapeutic delivery agent. This is entirely unexpected, as evidenced by the lack of testing of such unfunctionalized SANGs in any reported account of related materials: those skilled in the art would assume that functionalization would be necessary for selective delivery to tumor tissue. Remarkably, unfunctionalized SANGs can thereby function as a drug delivery platform that exhibits comparable performance among a variety of different cancer models, all of which share common properties of tortuous tumor vasculature but do not necessarily share common cell-surface markers. The term “unfunctionalized” generally refers to a material that is not augmented with a functional group to impart a specific property (e.g., tumor targeting) to the nanohydrogel. For example, the polymer shell can be prepared such that it does not have a targeting moiety selective for a specific tumor. In this regard, the resulting nanohydrogel can respond to a variety of different disorders. In some aspects, the nanohydrogel has a hydrodynamic size of from about 75 nm to about 200 nm prior to self-agglomeration.

In some aspects, the population of nanohydrogels is inducible via tortuous flow of an aberrant microvasculature of a tumor microenvironment (TME) to self-agglomerate to form an agglomerated cluster, wherein the agglomerated cluster has an average hydrodynamic size larger than an average hydrodynamic size prior to self-agglomeration.

In some aspects, self-agglomeration of the population of nanohydrogels is concentration-dependent. In some aspects, a concentration of nanohydrogels causing self-agglomeration is 10 nM or greater (e.g., 20 nM or greater, 30 nM or greater, 40 nM or greater, 50 nM or greater, 60 nM or greater, 70 nM or greater, 80 nM or greater, 90 nM or greater, 100 nM or greater, 110 nM or greater, 120 nM or greater, 130 nM or greater, 140 nM or greater, 150 nM or greater, 156 nM or greater, 200 nM or greater, 250 nM or greater, 500 nM or greater, 1 M or greater). In some aspects, a concentration of nanohydrogels causing self-agglomeration is from about 10 nM to about 1 M (e.g., from about 10 nM to about 500 nM, from about 30 nM to about 500 nM, from about 60 nM to about 500 nM, from about 60 nM to about 250 nM, from about 100 nM to about 250 nM, from about 100 nM to about 200 nM, or about 156 nM).

### Imaging Agents

Also described herein are compositions comprising: any of the populations of nanohydrogel described herein; and an imaging agent. In some aspects, the imaging agent is associated with (e.g., encapsulated within a core of, tethered to) the nanohydrogel.

Imaging agents (i.e., detectable labels) suitable for use in the compositions described herein include those which report by any suitable modality, including fluorescence, absorbance, radioactivity, or emission (e.g., hyper spectral emission). In certain embodiments, imaging agents (i.e., detectable labels) suitable for use in the compositions described herein include those which report by any suitable modality, including fluorescence, absorbance, radioactivity, contrast imaging (e.g., magnetic resonance imaging (MRI), positron emission tomography, computed tomography (CT)) or emission (e.g., hyper spectral emission). In certain embodiments, the imaging agent comprises a fluorophore, a near-infrared dye, a heavy metal, a radionuclide, barcoded oligonucleotide, a quantum dot, a contrast agent, a heavy metal, a metal complex, or a radionuclide. In some embodiments, the imaging agent comprises a fluorophore, a near-infrared dye, a heavy metal, a radionuclide, barcoded oligonucleotide, or a quantum dot. Use of barcoded oligonucleotides for nano-barcoding may be performed as described in the art, e.g., in PNAS 114(8): 2060-2065 (2017), which is hereby incorporated by reference in its entirety. Detection of compositions wherein the imaging agent is a barcoded oligonucleotide (e.g., barcoded oligo-DNA) is suitably by sequencing.

In other embodiments, the imaging agent comprises a contrast agent, a heavy metal, a metal complex, or a radionuclide. Suitable contrast agents include radiopaque agents that are iodinated or contain barium, and paramagnetic contrast agents that include gadolinium. Suitable metal complexes for use as imaging agents in MRI and CT methods include, but are not limited to, gadolinium-based contrast agents, such as chelates of gadolinium and chelates of manganese, including those known in the art.

Suitable radionuclides are preparable by attaching a radioactive atom to the nanohydrogel. Suitable radioactive atoms include those typically used in positron emission tomography imaging, including, but not limited to, ¹⁸F, ¹¹C, ¹³N, ¹⁵O, ⁶⁸Ga, ⁶⁴Cu, ⁵²Mn, ⁵⁵Co, ⁸⁹Zr, and ⁸²Rb. For example, the radioactive atom is ¹⁸F.

In certain embodiments, the imaging agent comprises a fluorophore. Suitable fluorophores include small molecules as well as fluorescent proteins and are suitably characterized by one or more emission wavelengths above those associated with background autofluorescence of biological samples, such as emission wavelengths above 500 nm.

Illustrative examples of molecules suitable for use as detectable labels in particular embodiments include, but are not limited to: Oregon Green®; Pacific Blue™; Pacific Orange™; Pacific Green™; Cascade Blue™; Cascade Yellow™; Lucifer Yellow™; Marina Blue™; Texas Red® (TxRed); AlexaFluor®(AF) dyes, e.g., AF350, AF405, AF488, AF500, AF514, AF532, AF546, AF555, AF568, AF594, AF610, AF633, AF635, AF647, AF680, AF700, AF710, AF750, AF790, and AF800; QDot® nanocrystals, e.g., Qdot®525, Qdot®565, Qdot®585, Qdot®605, Qdot®655, Qdot®705, and Qdot®800; DyLight™ Dyes (DL), e.g., DL549, DL649, DL680, and DL800; fluorescein or a derivative thereof, e.g., fluorescein isothiocyanate, carboxyfluorescein, and dichlorotriazinylamine fluorescein; digoxigenin; dinitrophenol (DNP); trinitrophenol (TNP); biotin; Cy dyes, e.g., Cy2, Cy3, Cy3.5, Cy5, Cy5.5, Cy7, and Cy 7.5; Phycoerythrin (PE, R-Phycoerythrin (RPE)); B-Phycoerythrin (BPE); Peridinin Chlorophyll (PerCP); Allophycocyanin (APC); C-Phycocyanin; Atto® Dyes, e.g., Atto 390, Atto 425, Atto 465, Atto 488, Atto 495, Atto 514Atto 520, Atto 532, Atto 550, Atto 565, Atto 590, Atto 594, Atto 610, Atto 620, Atto 633, Atto 647, Atto 655, Atto 665, Atto 680, Atto 700, Atto 725, and Atto 740; Super Bright™ Dyes, e.g., Super Bright™ 436, Super Bright™ 600, Super Bright™ 645, Super Bright™ 702, and Super Bright™ 780; Brilliant™ Dyes, e.g., Brilliant™ Violet 421, Brilliant™ Violet 480, Brilliant™ Violet 510, Brilliant™ Violet 605, Brilliant Violet™ 650, Brilliant Violet™ 711, Brilliant Violet™ 786, Brilliant™ Ultraviolet 395 (BUV395), Brilliant™ Ultraviolet 496 (BUV496), Brilliant™ Ultraviolet 563 (BUV563), Brilliant™ Ultraviolet 661 (BUV661), Brilliant™ Ultraviolet 737 (BUV737), Brilliant™ Ultraviolet 805 (BUV805), Brilliant™ Blue 515 (BB515), and Brilliant™ Blue 700 (BB700); and IR Dyes, e.g., IR Dye 680, IR Dye 680LT, IR Dye 700, IR Dye 700DX, IR Dye 800, IR Dye 800RS, and IR Dye 800CW.

Illustrative examples of tandem fluorescent dye molecules suitable for use as detectable labels include, but are not limited to: RPE-Cy5, RPE-Cy5.5, RPE-Cy7, RPE-CF594, RPE-AlexaFluor® tandem conjugates; RPE-Alexa610. RPE-TxRed, APC-H7, APC-R700, APC-Alexa600, APC-Alexa610, APC-Alexa750, APC-Cy5, APC-Cy5.5, and APC-Cy7.

Illustrative examples of fluorescent proteins suitable for use as detectable labels include, but are not limited to: GFP, eGFP, BFP, CFP, YFP, DsRed, DsRed2, mRFP, mBanana, mOrange, dTomato, tdTomato, mTangerine, mStrawberry, mCherry, mPlum, and mRaspberry.

In certain embodiments, SANGs can be functionalized or loaded with one or more of covalently attached fluorophores (e.g., Texas Red, AlexaFluor dyes) for optical imaging and for ex vivo detection t, chelators for radiometal labeling (e.g., DOTA, NOTA) for PET/SPECT contrast, paramagnetic agents (Gd-chelate conjugates, manganese complexes) or iron oxide nanoparticles embedded or attached for MRI contrast, echogenic or gas-forming moieties for ultrasound contrast where compatible, and non-covalent entrainment of nucleic acid labels or barcodes to enable downstream molecular detection of labeled cells (e.g., by qPCR after isolation).

In certain embodiments, one or more imaging agents are tethered to (i.e., conjugated, coupled, or linked (e.g., covalently bonded)) to the nanohydrogel. The imaging agents may be tethered to the nanohydrogel either directly or indirectly (e.g., via a linker group). A nanohydrogel can be directly covalently bound to one or more imaging agents when the nanohydrogel and the imaging agents each possess a substituent capable of reacting with the other. For example, a nucleophilic group, such as an amino group, on one (e.g., on the nanohydrogel) may be capable of reacting with a carbonyl-containing group, such as an anhydride or a succinimide, or with an alkyl group containing a good leaving group (e.g., a halide) on the other (e.g., on the imaging agent). In addition, several imaging agents bearing synthetic handles for such conjugation are commercially available, such as from LICORbio (https://www.licorbio.com/).

In certain embodiments, it may be desirable to couple, conjugate, or link a nanohydrogel to one or more imaging agents via a monovalent or polyvalent linker or a spacer. A linker or spacer can be used to provide sufficient distance between a nanohydrogel and an imaging agent to avoid steric hindrance. It will be evident to those skilled in the art that a variety of bifunctional or polyfunctional reagents, both homo- and hetero-functional (such as those described in the catalog of the Pierce Chemical Co., Rockford, IL), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

### Pharmaceutical Compositions

In certain embodiments, the invention provides for a pharmaceutical composition comprising SANGs as disclosed herein with a pharmaceutically acceptable diluent, carrier, solubilizer, emulsifier, preservative and/or adjuvant.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995).

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the SANGs.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising the SANGs can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising the SANGs can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising the SANGs in a pharmaceutically acceptable vehicle. In certain embodiments, a vehicle for parenteral injection is sterile distilled water in which the SANGs are formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

Additional pharmaceutical compositions will be evident to those skilled in the art, including formulations involving SANGs in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, the effective amount of a pharmaceutical composition comprising SANGs to be employed will depend, for example, upon the context and objectives. One skilled in the art will appreciate that the appropriate dosage levels for diagnostics, according to certain embodiments, will thus vary depending, in part, upon the molecule delivered, the indication for which SANGs, are being used, the route of administration, and the size (body weight, body surface or organ size) and/or condition (the age and general health) of the patient. In certain embodiments, the clinician can titer the dosage and modify the route of administration to obtain the optimal effect.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intraocular, intraarterial, intraportal, or intralesional routes; by sustained release systems or by implantation devices. In certain embodiments, the compositions can be administered by bolus injection or continuously by infusion, or by implantation device. In certain embodiments, the composition can be administered locally via implantation of a membrane, sponge or another appropriate material onto which the desired molecule has been absorbed or encapsulated.

For example, SANGs and SANGs bearing detectable moieties (e.g., SANGs labeled with imaging compatible moiety) may be suitably formulated for use in the methods of the present disclosure in physiologically acceptable carriers for systemic administration (e.g., intravenous). Dosing ranges and concentrations may be adapted per species and diagnostic purpose; example doses used in preclinical studies (e.g., mice, rats, and pigs) include about 1 mg·kg⁻¹ to about 7 mg·kg⁻¹.

### Biological Samples

In some embodiments, the method of detecting the solid tumor in the subject comprises the step of obtaining a biological sample from the subject. Suitable biological samples for use in the methods of the present disclosure include bodily liquids and fluids. In some embodiments, the biological sample is a blood, plasma, or serum sample. Suitably, the biological sample is obtainable from the subject non-invasively or minimally invasively, such as by a blood draw or a pin prick. Suitably, the obtaining the biological sample from the subject is performed by the subject themselves or by one skilled in the art, such as a medical professional.

### Methods of Surgical Debulking

In other aspects, described herein is a method of surgically debulking a solid tumor in a subject, comprising:
imaging the solid tumor in the subject with a nanohydrogel composition described herein; and
surgically debulking the solid tumor.

In still other aspects, described herein is a method of surgically debulking a solid tumor in a subject, comprising:
detecting the solid tumor in the subject, ex vivo, according to any of the methods described herein;
imaging the solid tumor in the subject with a nanohydrogel composition described herein; and
surgically debulking the solid tumor.

In some embodiments, the detecting the composition in the subject occurs while the subject is undergoing a surgical procedure. In some such embodiments, the surgical procedure is surgical removal or surgical debulking of the solid tumor. In some such embodiments, the method further comprises a step of, after the detecting the composition in the subject and thereby imaging the solid tumor, surgically debulking the solid tumor.

As used herein, “surgical debulking” of a solid tumor or “surgically debulking” a solid tumor means surgically removing a portion of the solid tumor (i.e., some but not all of it). For example, “surgical debulking” includes, but is not limited to, surgically removing at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the solid tumor. In some embodiments, the surgically debulking the solid tumor comprises surgically removing at least 80% of the solid tumor, at least 90% of the solid tumor, or at least 95% of the solid tumor.

Imaging the solid tumor in the subject may be by any suitable means of in vivo detection of fluorescence in a living subject, e.g., a human subject. For example, the imaging may be accomplished by use of a system or a device such as those described in U.S. Pat. No. 10,258,275 or U.S. Pat. No. 9,763,577, each of which is hereby incorporated by reference in its entirety. U.S. Pat. No. 9,763,577 describes, for example, a device comprising a lightweight handheld tool with a small profile to allow maneuverability and limit intrusiveness of the device in the operating room. In certain embodiments, of U.S. Pat. No. 9,763,577, the device employs white light illumination to deliver excitation photons to the specimen and a combination of spectral filters for fluorescence excitation and collection. The device of U.S. Pat. No. 9,763,577 may be connected to a standard computer, which collects, analyzes, and displays the resulting image for the physician. Without wishing to be bound to any particular theory, selective uptake of the SANGs (i.e., the SANG-tethered fluorophores) allows the physician (e.g., the surgeon) to distinguish cancer cells from healthy cells, thereby permitting the preferential removal of tumor cells and not healthy cells (beyond the level that may be desirable for margins). Suitably, the light source in the device is compatible with the excitation wavelength of the fluorophore.

In other embodiments, the method of surgically debulking comprises, prior to the imaging the solid tumor, administering to the subject a positron emission tomography (PET) imaging agent, such as a radionuclide, and the imaging the solid tumor comprises PET imaging. Suitable radionuclides are preparable by attaching a radioactive atom to the SANG. Suitable radioactive atoms include those typically used in PET imaging, including, but not limited to, ¹⁸F, ¹¹C, ¹³N, ¹⁵O, ⁶⁸Ga, ⁶⁴Cu, ⁵²Mn, ⁵⁵Co, ⁸⁹Zr, or ⁸²Rb, such as ¹⁸F. In some such embodiments, the step of surgically debulking the solid tumor may be assisted by the use of systems or devices described in the previous paragraph, i.e., devices such as those described in U.S. Pat. No. 9,763,577 relying on fluorescence.

### Kits

The disclosure also provides kits for use according to any of the methods described herein. Such kits can include one or more containers comprising any of the compositions described herein.

In some embodiments, the kit comprises instructions for preparing any of the compositions described herein. In some embodiments, the kit comprises instructions for use in accordance with any of the methods described herein. The included instructions can comprise a description of administration of the composition according to any of the methods described herein. The kit may further comprise a description of selecting an individual suitable for the methods described herein.

The instructions relating to the use of the composition described herein, generally include information as to dosage, dosing schedule, and route of administration for the intended method. The containers may be unit doses, bulk packages (e.g., multi-dose packages) or sub unit doses. Instructions supplied in the kits are typically written instructions on a label or package insert (e.g., a paper sheet included in the kit), but machine-readable instructions (e.g., instructions carried on a magnetic or optical storage disk) are also acceptable.

The kits as described herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, injector (e.g., syringe and needle), nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). The kits described herein may optionally provide additional components such as buffers and interpretive information. Normally, the kit comprises a container and a label or package insert(s) on or associated with the container. In some embodiments, the present disclosure provides articles of manufacture comprising contents of the kits described above.

In some embodiments, the kit comprises a vial or prefilled syringe of labeled SANG formulation with instructions for systemic administration (dose, recommended time-points). In some embodiments, the kit comprises blood collection materials and reagents for detection (flow cytometry antibodies, buffers, microfluidic chips, imaging slides). In some embodiments, the kit comprises positive and negative control cell lines or reference materials (to validate detection). In some embodiments, the kit comprises reagents for radiolabeling or MRI contrast agent conjugation prior to administration.

In certain embodiments, kits are provided for producing a single-dose administration unit. In certain embodiments, kits containing single and multi-chambered pre-filled syringes (e.g., liquid syringes and lyosyringes) are included.

### EXAMPLES

The following examples are set forth below to illustrate the compositions, methods, and results according to the disclosed subject matter. The examples are not intended to be inclusive of all aspects of the subject matter disclosed herein, but rather to illustrate representative methods and results. These examples are not intended to exclude equivalents and variations of the present invention which are apparent to one skilled in the art.

### Example 1: SANG Synthesis

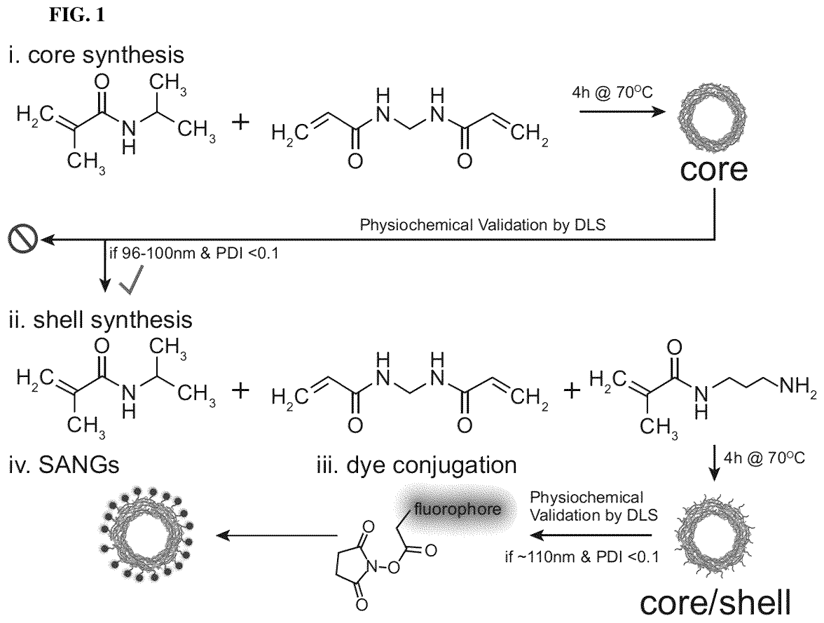

SANGs were synthesized as previously described. International Appl. No. PCT/US2024/040745; Housley et al., bioRxiv 2024.01.22.575714; doi: https://doi.org/10.1101/2024.01.22.575714 (2024); Housley et al., Tumor agnostic drug delivery with dynamic nanohydrogels. Nat Commun 17, 184 (2026). https://doi.org/10.1038/s41467-025-66788-4. Polyacrylamide-based core-shell nanohydrogels were made from inexpensive materials and were formulated as soft and biocompatible particles using a two-stage free-radical precipitation polymerization method first described by Lyon and coworkers (FIG. 1) (Blackburn, W. H., Dickerson, E. B., Smith, M. H., McDonald, J. F., & Lyon, L. A. (2009). Peptide-functionalized nanogels for targeted siRNA delivery. Bioconjugate chemistry, 20(5), 960-968; Jones, C. D., & Lyon, L. A. (2000). Synthesis and characterization of multiresponsive core-shell microgels. Macromolecules, 33(22), 8301-8306; Gan, D., & Lyon, L. A. (2001). Interfacial Nonradiative Energy Transfer in Responsive Core-Shell Hydrogel Nanoparticles. Journal of the American Chemical Society, 123(34), 8203-8209). The nanohydrogels (SANGs) were chemically functionalized only by the attachment of a small amount of fluorophore by amide bond formation. Physicochemical characterization has been previously reported. International Appl. No. PCT/US2024/040745; Housley et al., bioRxiv 2024.01.22.575714; doi: https://doi.org/10.1101/2024.01.22.575714 (2024).

### Example 2: Development of Large Animal Ovarian Cancer Model for Translational Nanoparticle Investigations

Abstract: The five-year survival of ovarian cancer (OC) remains low. While murine models are ubiquitously used, they may not faithfully mimic human OC. To better model the human condition, a large-animal model of OC was developed by expressing mutant KRAS and PIK3CA and shRNA-p53, three of the top four oncogenes observed in human ovarian carcinomas in sexually mature, immunocompetent female pigs. Animals were evaluated daily for clinical deficits for three months. CT scans revealed contrast enhancing mass lesions at the site of oncogenic lentiviral injection in three of four animals by one month which coincided with the detection of circulating tumor cells (CTCs) an emerging clinical hallmark of OC. At planned three-month endpoint, MRI scans demonstrated increased tumor size and CTCs detected in blood and ascites all without overt clinical presentation. Histopathologic features closely aligned with human high-grade ovarian tumors including high Ki-67 proliferative index. Two preliminary investigations were then performed to test their translational utility for use in drug and diagnostic tool development. Using our recently developed “self-agglomerating nanohydrogels” (SANGs), the generalizability of SANGs' tissue- and species-agnostic tumor targeting was assessed. Previously demonstrated to target ovarian, colorectal, breast, and lung tumors in mice and rats, after intravenous injections, SANGs were uniformly detected in primary tumors as compared to non-malignant tissues. SANGs were then found to be highly expressed intracellularly in CTCs relative to non-malignant circulating cells, suggesting their potential as a tool to aid in the diagnosis of solid tumor cancers. Although further optimization is warranted, this study describes the first immunocompetent model of OC in higher order species and demonstrates its potential utility for focused research and development of diagnostic and therapeutic technologies for OC.

### Introduction

Ovarian cancer (OC) remains one of the most lethal gynecological malignancies due to its asymptomatic nature and late-stage diagnosis. While murine models are ubiquitously used and they have produced tremendous advances in the understanding and treatment of cancer, the fact remains that only a few percent of diagnostic or drug candidates identified in preclinical studies successfully translate to clinical practice (Sharpless and Depinho, 2006; Kola and Landis, 2004; Reichert and Wenger, 2008; Singh et al., 2012). Murine models may not faithfully mimic human OC and response to interventions, potentially due to interspecies differences in physiology, tumor microenvironment, and genetics. Model inaccuracy may contribute to the low drug approval rate for potential cancer therapeutics and may impede diagnostic or medical device development. These limitation underscores the need for more representative large animal models that can bridge the gap between preclinical studies and human clinical trials.

With greater similarity to humans with respect to size, anatomy, physiology, pathophysiological responses, and sequence homology than do mice, pigs have demonstrated exceptional translational value in the fields of trauma, transplantation, and cardiovascular disease. Pigs could have greater accuracy in cancer modeling than do mice. Importantly, a porcine OC model would permit research and development of therapeutics, imaging instrumentation, and interventional devices suitable for rapid translation to human OC patients. Currently, there are no validated large-animal models for OC. Although some proof-of-principle studies in pig have been published, they utilized either immunodeficient or sexually immature piglets to grow human OC cells both of which complicate their utility. While the recently developed transgenic ‘Oncopig’ line has successfully modeled solid tumors with organ specificity, they carry only two oncogenic mutations. As a result, Oncopigs lack the flexibility to model the molecular diversity observed in human cancer.

Here it is reported the development of a large-animal model of OC by expressing three of the top four oncogenes observed in human OC in sexually mature, immunocompetent female pigs. Clinical imaging and microfluidic based whole blood analyses track tumorigenesis and reveals the coincident detection of circulating tumor cells (CTCs), an emerging clinical hallmark of OC throughout the three-month study. Pig tumor histopathological features were benchmarked against our biorepository of human high-grade ovarian tumors. Two preliminary were then performed investigations to test the model's translational utility for use in drug and diagnostic tool development. After intravenous injection, the recently developed tumor targeting “self-agglomerating nanohydrogels” (SANGs), were uniformly detected in primary tumors as compared to non-malignant tissues. SANGs were then found to be highly expressed intracellularly in CTCs relative to non-malignant circulating cells, suggesting their potential as a tool to aid in the diagnosis of solid tumor cancers. This study describes the first immunocompetent model of OC in higher order species and demonstrates its potential utility for focused research and development of diagnostic and therapeutic technologies for OC. The SANGs platform is further credentialed as a species agnostic tool for targeting solid tumor cancers. Finally, this report provides the first demonstration of in vivo use of a nanoparticle system to identify CTCs opening the door for SANGs' potential application as a tool to aid in the diagnosis of solid tumor cancers.

### Results

### Delivery of Top Ovarian Cancer Oncogenes to Immunocompetent, Sexually Mature Pigs.

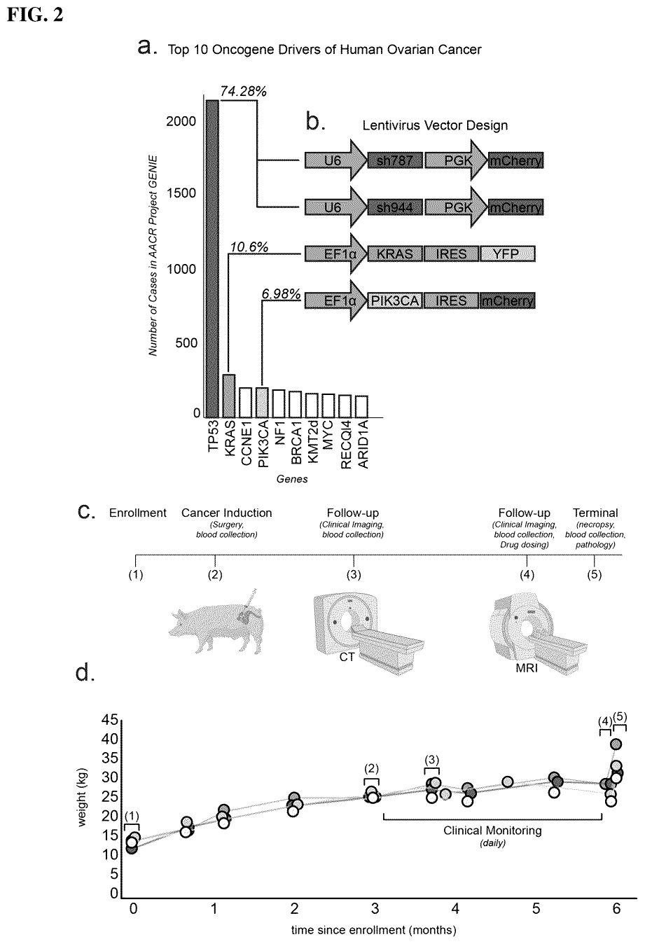

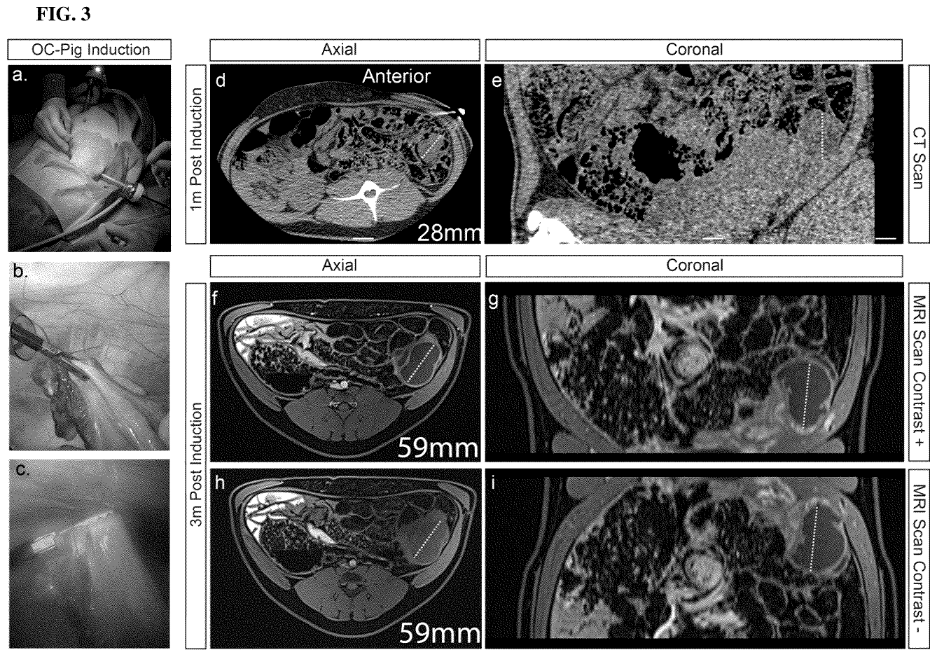

Unilaterally intraovarian injections (50 μL) of a lentiviral vector cocktail were performed in sexually mature, immunocompetent, treatment naïve female Gottingen pigs (25.75±1.25 kg, n=4, FIG. 2, panel c). Vector cocktails contained a 1:1:1 ratio of activating KRAS (G12V) and PIK3CA (H1047R) and two sequences of shRNA targeting the porcine p53 (FIG. 2, panel a, vectors titered at >10⁹IU·mL⁻¹), three of the top four oncogenes observed in human ovarian carcinomas (The My Cancer Genome®). Ovaries were identified either laparoscopically prior to injection (FIG. 3, panels a-c, n=3) or after externalization through a small (~7 cm) horizontal incision in the inferior abdominal wall as the ovary could not be identified and manipulated to successfully perform a percutaneous injection. Blood collected after surgery revealed no changes in blood serum chemistry relative to pre-operative levels indicating minimal systemic response to surgery.

### CT and MR Imaging Detect Tumorigenesis Despite Asymptomatic Clinical Presentation.

After recovery from surgery, animals were evaluated daily for clinical deficits for three months (planned endpoint). Computed tomography (CT) imaging was performed at approximately one month and MRI at endpoint and collected whole blood at each imaging session. All animals remained bright, alert, and responsive, with no signs of weight loss, cachexia, neurologic symptoms or changes bowel and bladder function. Four-five weeks after vector cocktail delivery (FIG. 2, panel c), CT imaging revealed contrast enhancing mass lesions at the site of oncogenic lentiviral injection in three of four animals (FIG. 3, panels d-e), suggestive of successful ovarian cancer induction. At endpoint, three months after vector cocktail delivery (FIG. 2, panel c), animals underwent MRI prior to euthanasia and necropsy. MRI demonstrated T1w contrast enhancing mass forming lesions with marked peri-lesional edema at the site of oncogenic lentiviral injection in all animals (FIG. 3, panels f-i). These radiologic features are consistent with ovarian carcinoma and included maximal tumor diameters ranging from 30-59 mm (n=4). Given all animals remained asymptomatic throughout the study, this collective presentation is consistent with human ovarian cancer which frequently remains covert despite advanced tumorigenesis detectable by clinical imaging.

### Histopathological and Immunohistochemical Characterization.

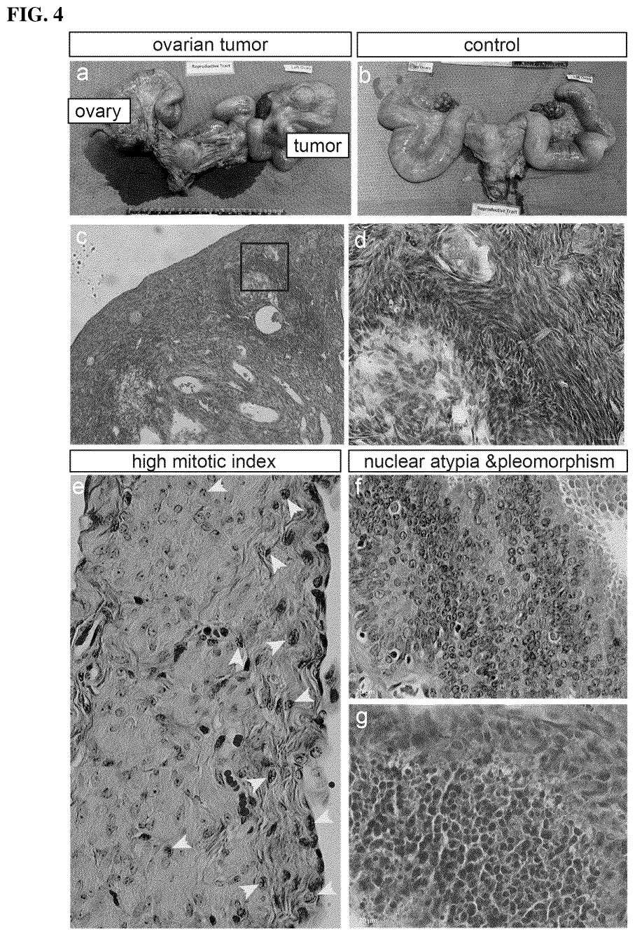

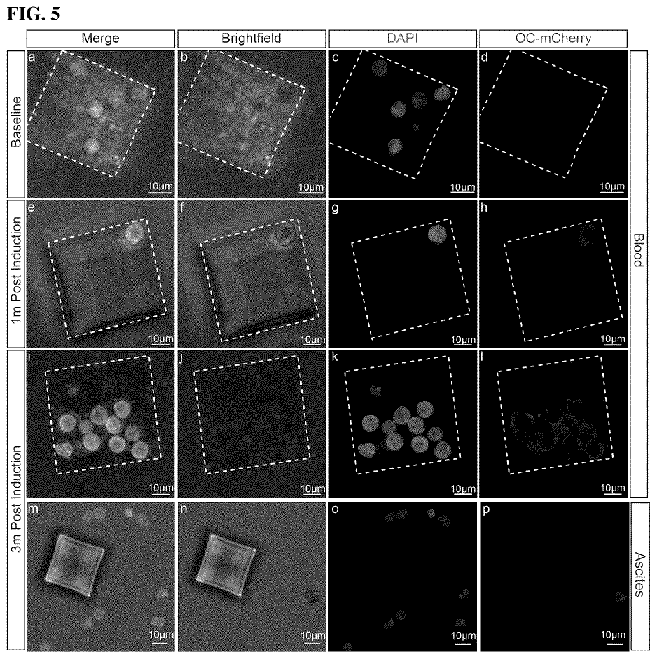

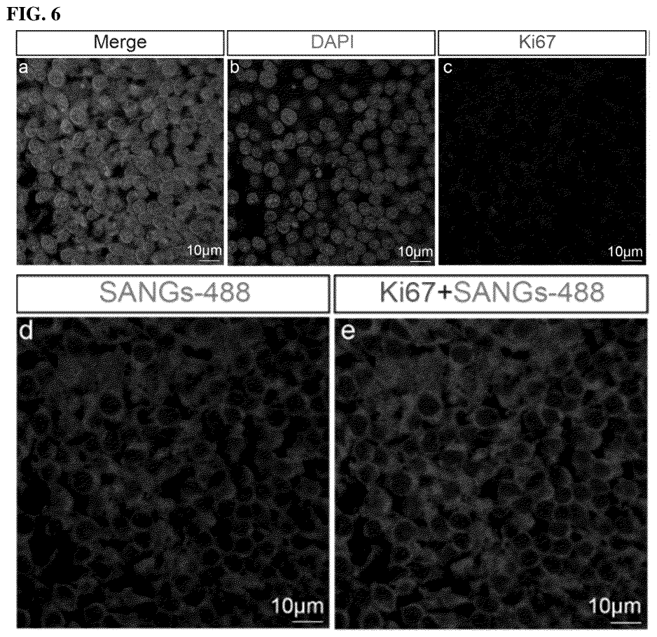

86-89 days after induction, all animals underwent planned euthanasia and necropsy 1-2 days post MRI after uncomplicated courses. Samples of abdominal fluid were collected for further analysis (see below). Gross evidence of tumors were detected ipsilateral to vector injection. Normal ovaries were noted contralateral to vector injection (FIG. 4, panel a) and were indistinguishable from ovaries from control animals (FIG. 4, panel b). Hematoxylin and Eosin (H&E) staining of slices ipsilateral to the viral vector delivery demonstrated abundant tumor cells in all subjects (FIG. 4, panel c). At low magnification, the tumors demonstrated cellular regions showing hyperchromasia and SET (solid, pseudoendometrioid or transitional) pattern (FIG. 4, panels c-d). Histopathologic features at high magnification include severe nuclear atypia and significant nuclear pleomorphism (3× variation in size), with large, peculiar, and multinucleated forms. Prominent nucleoli can be seen, with a high mitotic index (≥12 mitotic figures per 10 high power fields). Identified histopathologic features are consistent with those observe in human ovarian carcinomas (FIG. 4, panels e-g). Histopathologic features of ovaries contralateral to injection site (control) revealed grossly normal medulla and cortex containing developing follicles. Normal corpus luteum and ovarian surface epithelium composed of a single cell lining was also noted throughout (data not shown).

Sections sequential to those studied with H&E were stained with anti-mCherry antibodies to directly detect virally transfected oncogenes. Strong mCherry expression was detected throughout tumor sections and was not detectable above background in control ovaries (data not shown). Sections sequential to those studied with H&E were stained with anti-Ki-67 antibodies and demonstrated strong immunopositivity with mean proliferative index of 70% which corresponded with human ovarian cancer sections and was significantly elevated relative to that observed in control ovaries 3% (data not shown).

Circulating Tumor Cells Detection Coincides with Tumorigenesis.

An emerging clinical hallmark of ovarian cancer is the presence of circulating tumor cells (CTCs). The presence of CTCs correlates with tumor stage, disease recurrence, and survival and has the potential to act as a liquid biopsy for drug treatment monitoring or as a companion tool to aid in diagnosis. Whether CTCs could be detected from whole blood samples was therefore tested over the course of the three-month study. Using the mCherry fluorescent reporter that was co-transfected with each vector cocktail, the presence or absence of CTCs was assessed relative to other circulating cell populations based on their mCherry expression. Whole blood was passed through a custom microwell filter with diagonal aperture of ~11 μm. This enabled large diameter circulating cells and cell clusters to be retained while smaller cell populations were depleted. While not specific for cancer cells, this form of size exclusion filtering enriches each sample for large diameter populations and enables high-throughput- and confocal-microscopy to image rare circulating cell types that might be lost to cytometry-based techniques. For consistency, −2 ml of whole blood was tested at each time point to mirror the volume collected during a typical clinical blood draw.

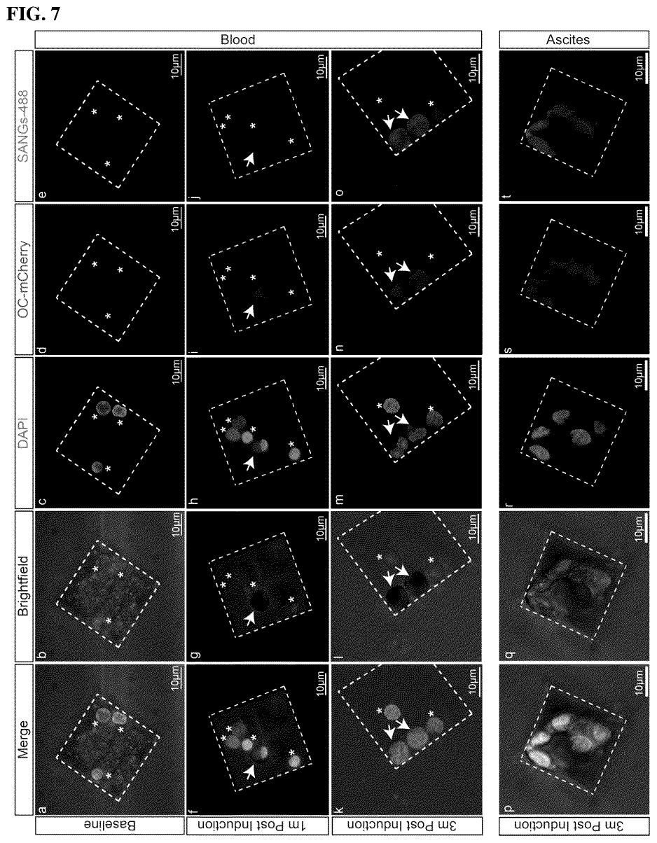

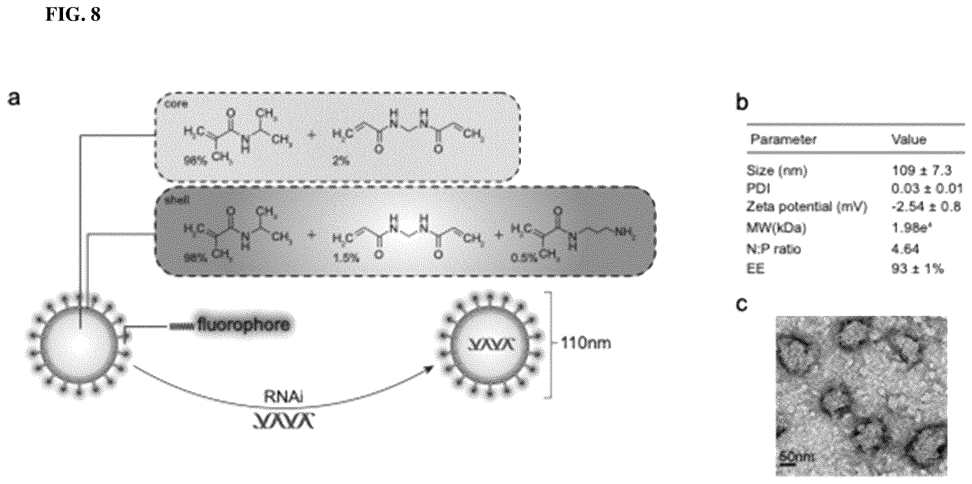

Analysis of whole blood collected at the time of surgery revealed numerous circulating cells were retained on the filter, yet no mCherry expression (FIG. 5, panels a-d) was detected. Confirmatory analysis was performed by fixing and staining filters with anti-mCherry antibodies followed by secondary antibody detection. Follow up imaging confirmed the expected absence of virally transfected cells and indicates that the collected cells were likely immune. Blood collected at the time of CT imaging was then evaluated, which was ~1 month later than the baseline. As shown in FIG. 5, panels e-h, sparse mCherry expressing cells were detected in each sample. These presumptive CTCs coincided with the detection of contrast enhancing mass lesions on CT. A larger number of mCherry expressing cells were detected in blood collected at end point which was ~3 months after the baseline (FIG. 5, panels i-1), possibly indicating the presence of CTC clusters due to their close association. This apparent increase in CTC number was notable as it coincided with the MRI indicated increased tumor volume (FIG. 3). mCherry positive, presumptive ovarian cancer cells were also detected in the ascites fluid collected at necropsy (FIG. 5, panels m-p). These data indicate that this large animal model recapitulates another key feature of human ovarian cancer.

Taken together, the clinical imaging, gross anatomical and histopathological tumor characteristics, and evidence of hematogenous and transcoelomic cancer cell dissemination help to credential our large animal model of ovarian cancer herein referred to as OC-Pigs. Moreover, this opens the possibility for its use as a translational model for use in drug and/or diagnostic tool development (preliminary use cases investigated below).

### Investigating the SANG Tumor Targeting Delivery Platform.

A nanohydrogel-based drug delivery platform was recently developed that preferentially targets and is retained in solid tumors. A recent study identified a new mechanism of action that promote selective distribution to tumors. This newly described environmentally responsive behavior leads to designation of these materials as “self-agglomerating nanohydrogels” (SANGs) as they achieve robust tumor delivery through concentration-dependent agglomeration that emerges uniquely in tumor microenvironments (TME). OC-Pigs provided a new model to test the generalizability of SANGs and provided the first testing bed to explore their translational utility.

Given the few animals available, a complete biodistribution and drug delivery study could not be performed. Instead, testing focused on if i.v. injected SANGs entrained in OC-Pig tumors as was found it does in rats and mice with ovarian, lung, breast, and colon cancer. Fluorescently decorated SANGs were formulated at 1 mg·kg⁻¹ in 3 ml of sterile saline then i.v. injected through a jugular catheter 72 hours prior to euthanasia in each OC-Pig. Sequential tissue sections not used for histopathology were examined by confocal fluorescence microscopy. SANGs were found distributed throughout ovarian tumor sections and deep within tumor parenchyma, exhibiting an extravasated distribution into the tumor interstitium (FIG. 6). SANG fluorescence was then compared in non-cancerous tissues, keeping all imaging parameters consistent to enable direct comparisons. Significantly lower SANG fluorescence was found in the contralateral ovary (control), liver, kidney, and spleen (FIG. 6) relative to OC-Pig tumors. These data indicate that SANGs successfully target, extravasate, penetrate tumor microenvironments, and gain access to the specific cells of interest in OC-Pig tumors. Moreover, these data further validate the species- and tumor-agnostic capabilities of the SANGs platform as this is their first demonstration of successful tumor targeting in a large animal model of cancer.

### Investigating SANGs as a Label Free Method for CTC Identification In Vivo.

Realizing the potential of CTCs as a diagnostic tool faces several challenges. Identifying CTCs typically relies on detecting unique receptor expression. Unfortunately, receptor expression is not necessarily uniform across multiclonal tumors and not conserved across time as cancer cells rapidly adapt to host and treatment pressures, leading to unacceptable false negative rates.

Given SANGs robustly target OC-Pig cancer cells in vivo and do not depend on ligand-receptor interactions, it was investigated if SANG expression could be detected in circulating cell populations and if so, could it be used as a method for differentiating CTCs from other circulating cell populations. SANG fluorescent expression was measured in captured cells from each blood sample passed through our custom microwell filter assay. The focus was on samples collected (1) prior to induction, (2) one-month post induction (coincident with CT), and (3) three-month post-induction, 72 hours after SANG treatment (at euthanasia).

At baseline prior to induction, abundant circulating cells devoid of mCherry expression and no SANG fluorescence were found (FIG. 7, panels a-e). As before, mCherry expressing cells were detected 1 month after induction when contrast enhancing lesions were detected on CT (FIG. 7, panels f-j). OC-Pig CTCs were frequently associated with other circulating cells and were presumptive immune cells. As expected, SANG fluorescence remains absent from both CTC and non-virally transfected cells indicating low background fluorescence. Seventy-two hours after SANG injection (end point), mCherry expressing and non-expressing cells continued to be found in close proximity (FIG. 7, panels k-o). Significantly higher SANG fluorescence was observed in mCherry expressing CTCs compared to their closely associated circulating immune cells. A similar pattern was observed in mCherry expressing cells found in ascites (FIG. 7, panels p-t). These data provide the first indication that SANGs have potential utility as a means to differentiate CTCs from non-malignant cells in circulation. More broadly, this is the first demonstration of in vivo use of a nanoparticle system to identify CTCs. It opens the door for SANGs' potential application as a tool to aid in the diagnosis of solid tumor cancers.

### Materials and Methods

### Animal Care

All procedures were conducted according to the Translational, Testing and Training Laboratories, Inc. —T3 Labs standard operating procedures and ethical guidelines of the approve Institutional Animal Care and Use Committee protocol (GT102P). Four sexually mature (10.6 months at surgery), immunocompetent, treatment naïve female Gottingen pigs (25.75±1.25 kg at surgery) were used in the present study. Animals were housed individually but with contact through the pen grating, in order to prevent wound and catheter cannibalism. Animals were fed ad libitum with standard feed (Purina Nature's Match® Sow and Pig Complete Feed) until the night before the surgical induction, when food was withheld.

FIG. 2 outlines the basic experimental design which included a ≥1 week acclimatization period after delivery to the research facility. Each subject then underwent an induction procedure (one major survival procedure), followed by observation for up to 3 months. This observation period included daily physical examinations that were performed in coordination with veterinary staff throughout acclimatization and post-operative assessments until a pre-determined 3-month endpoint, CT imaging with and without contrast under anesthesia at 1 month, and end point MRI with and without contrast. Blood was collected at each imaging time point.

### Vector Design

Vectors were designed separately maintain transduction efficiency and in accordance with biosafety requirements (29). A combination of third-generation replication deficient lentiviral vectors was used containing 1:1:1 ratio of activating Pik3ca (H1047R), KRAS (G12V), both produced on a pCDH transfer plasmid backbone under an Ef1α promoter, and a pLKO1 backbone expressing two sequences of shRNA targeting p53 including sh787 and sh944 under the H1 and U6 promoters. Vectors are titered at >10⁹IU·mL⁻¹ All vectors were thawed and combined in equal parts immediately prior to inoculation.

### Surgery

Animals preoperatively received atropine (Med-Pharmex Inc., Pomona, CA, USA) 0.04 mg/kg i.m., to reduce vagal effect, telazol (Zoetis Inc., Kalamazoo, MI, USA) 4 mg/kg i.m. in combination with xylazine (Akorn Inc., Marietta, GA, USA) 0.5 mg/kg i.m. for anesthesia induction, and buprenorphine (Patterson Inc., Phoenix, AZ, USA) 0.01 mg/kg i.m., for preoperative analgesia. After endotracheal intubation, anesthesia was maintained with 2-2.5% isoflurane (Abbot Labs, Chicago, IL, USA) in oxygen throughout all procedures.

### In Vivo Imaging: CT and MRI

Prior to either MRI or CT imaging, animals were sedated using an intramuscular injection of a mixture of ketamine (10 mg/kg) and xylazine (2 mg/kg). General anesthesia was induced and maintained with isoflurane (2-3%) delivered via an endotracheal tube. Vital signs, including heart rate, respiratory rate, and oxygen saturation, were continuously monitored throughout the procedure. Jugular catheters were placed acutely for blood sampling and delivery of contrast agents. The swine were positioned in dorsal recumbency on the table, and immobilization aids were used to ensure consistent positioning.

A multidetector CT scanner (GE) was used for imaging. The scanner was calibrated prior to each session, and the acquisition parameters were set as follows: Voltage: 120 kVp, Current: 150 mA Slice Thickness at 1 mm. Baseline imaging was performed without contrast. Scans were acquired to visualize abdominal cavity. The region of interest (ROI) included the ovaries, adjacent reproductive organs, and vascular structures. After non-contrast imaging, Gadolinium contrast agent (e.g., 30 mg/kg) was administered via an jugular catheter. A bolus injection of the contrast agent was delivered at a dose of 2 mL/kg using a power injector at a flow rate of 3 mL/s, followed by a saline flush. Dynamic imaging was initiated immediately after contrast administration to capture arterial, venous, and delayed phases of enhancement.

Magnetic Resonance Imaging (MRI) was performed on a 3T Siemens Prisma MRI at GSU/GT Center for Advanced Brain Imaging, Atlanta, GA. The animals were placed in the Head-First Supine position using Spine and Flex-coils. Eighteen axial T2-weighted images were acquired using motion-insensitive BLADE technique and STIR fat-suppression with 1.2×1.2×4 mm³ resolution, TR=4670 ms, TE=53 ms, TI=220 ms, Flip-angle=137 deg., TA=4:42 ms. Sixty axial T1-weighted images with Dixon fat-suppression technique were acquired with 0.8×0.8×2 mm³ resolution, TR=5.4 ms, TE1=2.46 ms, TE2=3.69 ms, Flip-angle=9 deg, TA=2:07m, pre- and post-contrast. Separate Fat and Water images were generated.

After imaging, anesthesia was discontinued, and animals were monitored during recovery. Post-procedure analgesia (e.g., meloxicam 0.2 mg/kg) was administered as needed, and animals were returned to their housing after complete recovery.

### Necropsy

Animals were deeply anesthetized then euthanatized with i.v. KCl (Hospira Inc., Lake Forest, IL, USA) mEq/kg i.v. After gross anatomical evaluation, tissues were fixed in 10% formalin, grossly sliced at 5 mm intervals for paraffin embedding.

### Microscopy: Tissues

Hematoxylin and Eosin staining (H&E) was performed in a standard fashion (Hematoxylin Gill No. 3, Sigma Aldrich, Cat: GHS332; Eosin Y, Sigma Aldrich, Cat: HT110132) on 10 μm thick tissue sections. H&E sections were evaluated by board-certified clinical pathologist. Immunohistochemistry (IHC) was performed with primary antibodies for Ki-67 (DSHB, AB_2617702) and mCherry (632496 Living Colors@DsRed Polyclonal Antibody). For H&E, whole slides were scanned at 20× magnification with Zeiss Axio Observer Z1 Color/Fluorescent Microscope equipment with a Zeiss AxioCam color camera and Hamamatsu Flash 4.0 CMOS camera. High magnification images were acquired with air 40×/1.3 NA LD Plan Neofluar (wd=0.210 mm), oil immersion 63×/1.4 NA Plan Apochromat (wd=0.190 mm) or oil immersion 100×/1.4 NA Plan Apochromat (oil) (wd=0.170 mm).

Immunofluorescence images were acquired with laser scanning confocal microscopy (LSCM) (Zeiss Biosystems, 900). Tile scans images of large tissue or well areas were acquired with a 20× objective (N.A.=0.8). Z stacks of tissue sections or high-resolution cell imaging were acquired with a 63× objective (N.A.=1.4) at a step size of 0.5 m. Laser strength, gain, and other critical image acquisition parameters were help constant when imaging across treatment groups. Stacks of images were processed and analyzed using Imaris (Bitplane) or imageJ (NIH) imaging software. Figures present images as either flat maximal projections of the sum of the z-axis optical slices. Analyses were performed by an investigator blinded to treatment group and was determined by measuring the mean fluorescence intensity observed on the flat maximal projections of the z-stacks images respectively.

### CTC Processing

Blood samples were collected in EDTA tubes (BD Vacutainer) and processed within 4 h of blood draw. To prevent sedimentation, tubes were placed on a rocker until use. Ascites samples were collected in evacuated glass containers and processed within 4 h of withdrawal.

A custom version of microwell filters with opening size of 11 microns were placed inside the filter holders and wetted with 1×PBS. Both ascites and whole blood samples were run through the devices using a syringe pump under withdrawal mode at 10 mL/h flow rate. The captured cells were fixed with 4% paraformaldehyde (Electron Microscopy Sciences, Hatfield, PA) for 10 min. The nuclei of cells were stained with 4′,6-diamidino-2-phenylindole (DAPI) (Invitrogen—Cat No: D1306, (1:1000)) for 10 min, and the devices were washed with 1×PBS. Lastly, devices were removed from the filter holders and mounted between two glass slides for imaging.

For confirmatory analysis, cells were permeabilized with 0.3% Triton-X (Sigma-Aldrich, St. Louis, MO) in PBS for 10 min. Prior to immunofluorescence staining, cells were incubated with a blocking buffer containing 5% goat serum for 60 min. Samples were incubated with anti-mCherry (632496 Living Colors® DsRed Polyclonal Antibody) overnight. Filters were washed again 3×in 0.01M PBS and immunoreactive sites were revealed with species specific fluorescent-conjugated secondary antibodies (Jackson ImmunoResearch Laboratories). Following a 2 hr incubation in the secondary mixture at room temperature, filters were washed 3×in 0.01M PBS, mounted, and coverslipped with Vectashield (Vector Labs, Newark, CA, H1000).

Immunofluorescence images were acquired with laser scanning confocal microscopy (LSCM) (Zeiss Biosystems, 900). Tile scans images of filters were acquired with a 20× objective (N.A.=0.8). Z stacks of high-resolution imaging were acquired with a 63× objective (N.A.=1.4) at a step size of 0.5 m. Laser strength, gain, and other critical image acquisition parameters were held constant when imaging across samples. Stacks of images were processed and analyzed using Imaris (Bitplane) or imageJ (NIH) imaging software. Figures present images as either flat maximal projections of the sum of the z-axis optical slices. Analyses were performed by an investigator blinded to treatment group and was determined by measuring the mean fluorescence intensity observed on the flat maximal projections of the z-stacks images respectively.

### Example 3: Tumor Agnostic Drug Delivery with Self-Agglomerating Nanohydrogels (SANGs)

RNA interference (RNAi) uses small interfering RNA (siRNA) and microRNA (miRNA) to silence expression of target genes in a sequence-specific manner(1, 2). RNAi molecules hold unique potential as clinically viable modalities for manifold diseases(3, 4). Oncological indications are of particular interest because the vast majority of cancer-promoting genes are “undruggable” (4). In order to reach its potential, RNAi molecules must be delivered to tumors, preferably after systemic injection. However, successful delivery of oligonucleotides is challenging due to their rapid clearance, nuclease susceptibility, and inability to traverse plasma membranes, resulting in insufficient localization to, and inadequate penetration of, tumors(3, 5, 6).

Numerous strategies have been developed to overcome these barriers, dominated by complexation with cationic polymers, inclusion within liposomes to form lipid nanoparticles (LNPs), or chemical modification (5, 7-11). While these strategies have improved delivery, trafficking is poorly understood and LNPs have drawbacks(12-15). Notably, the vast majority of LNP doses become trapped in the liver and are not efficiently taken up by cancerous cells after systemic injection(16-18). This leaves the majority of cancers untreated, especially metastases(16). Cationic polymers face different challenges: they bind nonspecifically to many cells types(19) and interact with serum components, resulting in short circulation times and toxic side effects. Surface shielding by PEGylation improves stability, reduces nonspecific binding and clearance, and prolongs circulation, but it also impairs target cell uptake(20, 21). While ligand functionalization can improve specificity, it increases manufacturing complexity and costs, reduces stability, and requires prior knowledge of the cell type of interest(16, 17). Carrier free delivery of siRNA can be achieved through chemical modification which drastically improves in vivo stability. While this has led to several FDA approved drugs, none have oncological indications(5). Collectively, the development of a systemic, tumor agnostic delivery system that is effective, safe, and simple to manufacture remains an important missing link for therapeutic translation of RNAi.

Described herein is the development of self-agglomerating nanohydrogels (SANGs) as a platform that achieves durable solid-tumor delivery without direct functionalization with targeting ligands. It is demonstrated that SANGs enable efficient delivery of RNAi molecules in vitro and exhibit preferential accumulation and retention in primary and metastatic loci in four cancer models in a species-agnostic manner, sensitizing previously drug-resistant tumors. SANGs were found to be safe and well tolerated in simulated clinical applications across three species. A provisional mechanistic proposal to explain how SANGs operate is provided, which takes advantage of an emergent property of these materials in vivo. Finally, the first direct observation of this emergent materials property in naturalistic conditions at the nanoscale is provided.

### SANGs Synthesis and Characterization

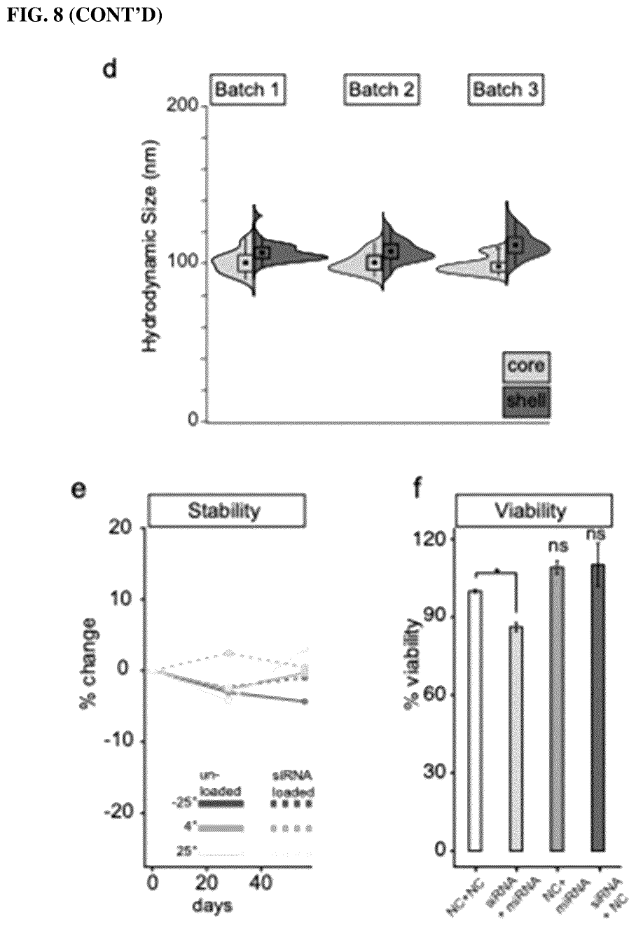

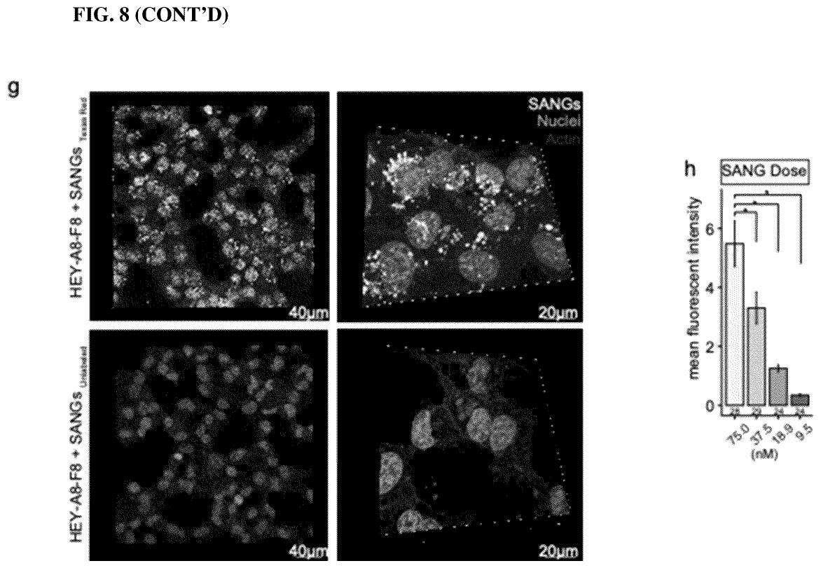

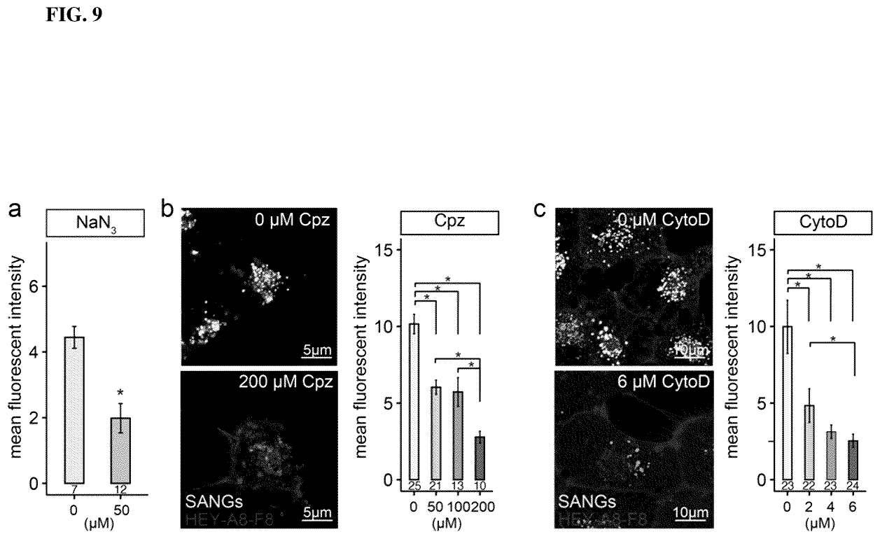

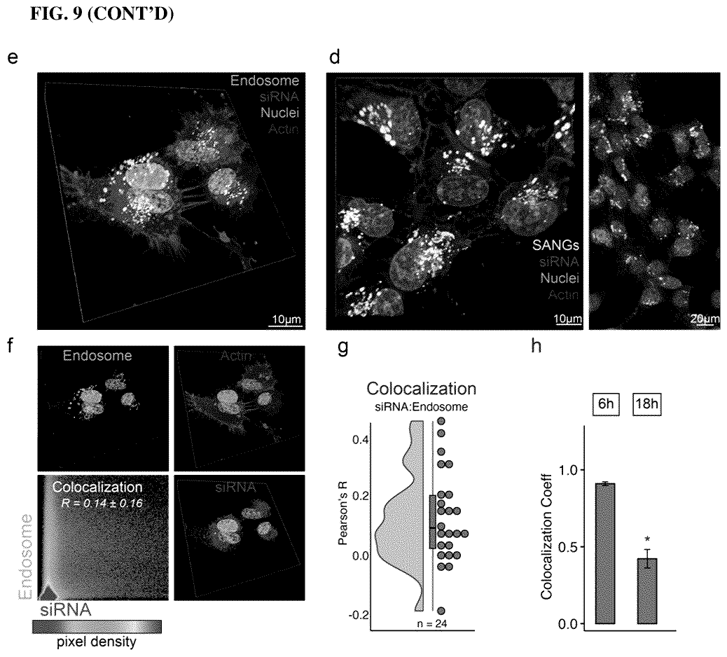

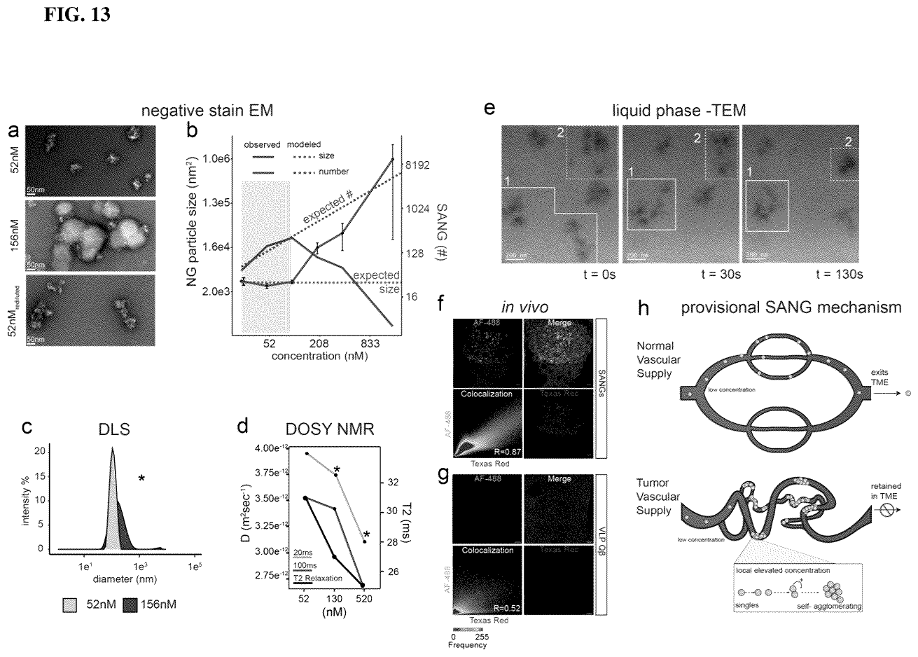

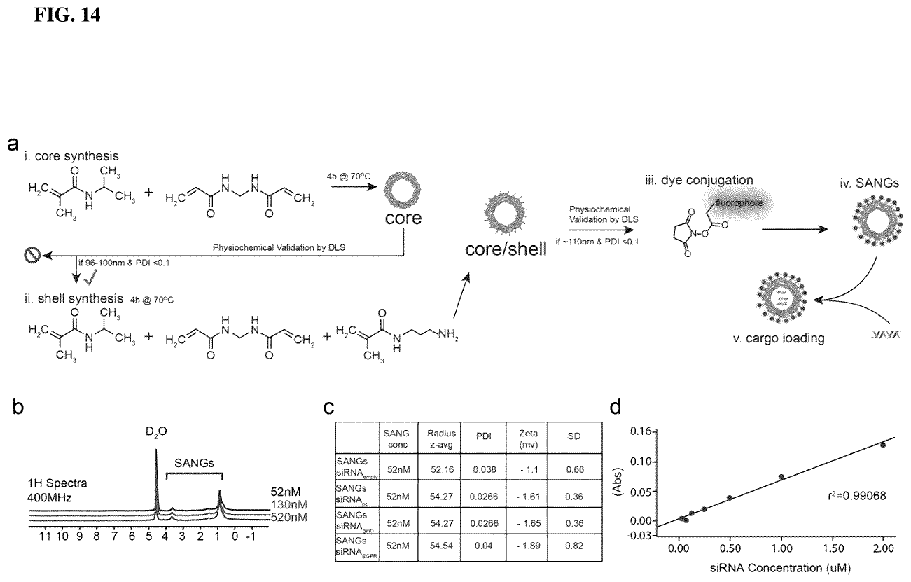

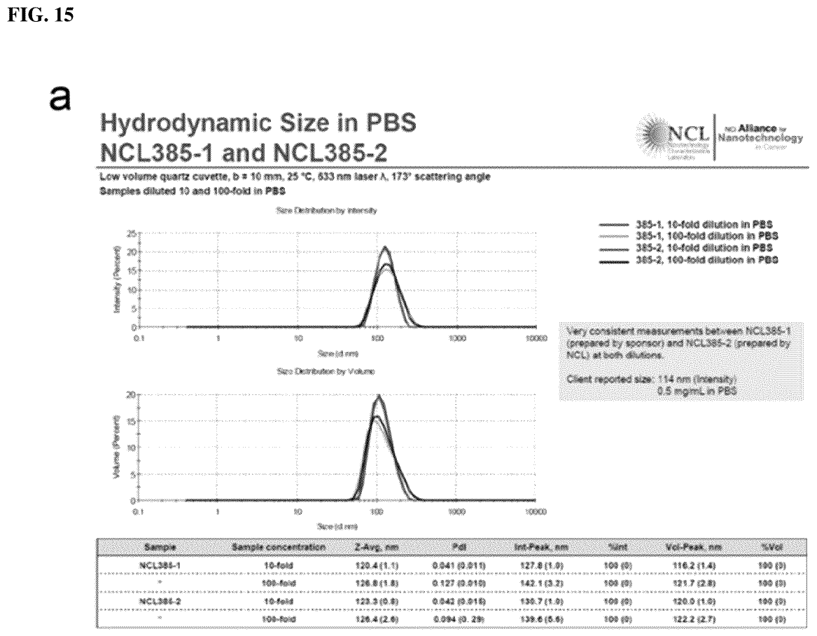

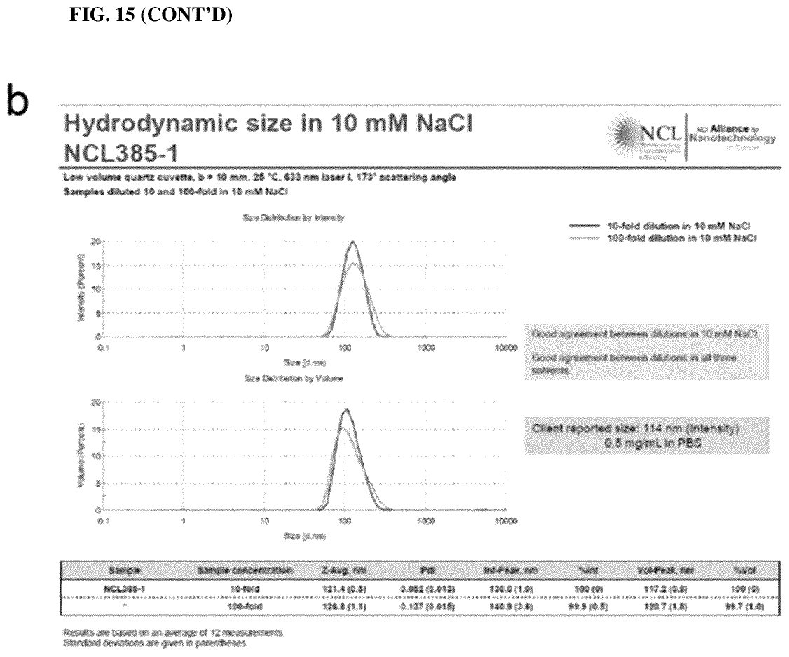

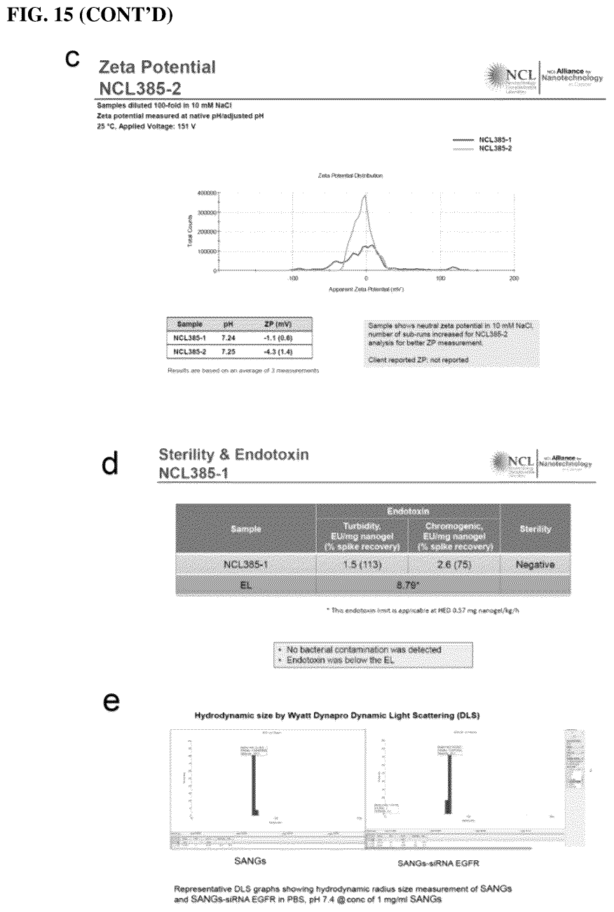

Polyacrylamide-based core-shell nanohydrogels are made from inexpensive materials and are formulated as soft and biocompatible particles using a simple, two-stage free-radical precipitation polymerization method first described by Lyon and coworkers (FIG. 8, panel a and FIG. 14)(22-24). siRNA can be easily loaded into these materials by swelling lyophilized particles in RNA-containing buffer; uptake of the RNA into the particles is complete and irreversible upon standing, although the spatial distribution of RNA cargo within the core-shell structure has not been characterized. It was previously observed that nanohydrogels functionalized with tumor-targeting peptides exclusively targeted ovarian tumors(25). However, instead of functionalizing the shell layer with directing ligands, the capabilities of untargeted nanohydrogels (SANGs), chemically functionalized only by the attachment of a small amount of fluorophore by amide bond formation were explored. Physicochemical properties were characterized (FIG. 8, panels b-c and FIG. 14) and independently validated by the National Characterization Laboratory under a variety of solvent, pH, dilutions, and temperature conditions (FIG. 8, panel d and FIG. 15). Both siRNA-loaded and unloaded SANGs were found to be unchanged in size, morphology, and concentration upon storage at −25 °C, 4 °C, and 25 °C out to 60 days in aqueous conditions (FIG. 8, panel e). SANGs prepared with negative control (NC) and scrambled RNAi molecules were shown to have no significant effect on the viability of HEY-A8-F8 cells (75 nM in particles, FIG. 8, panel f).

### SANGs Undergo Endosomal Uptake and Escape

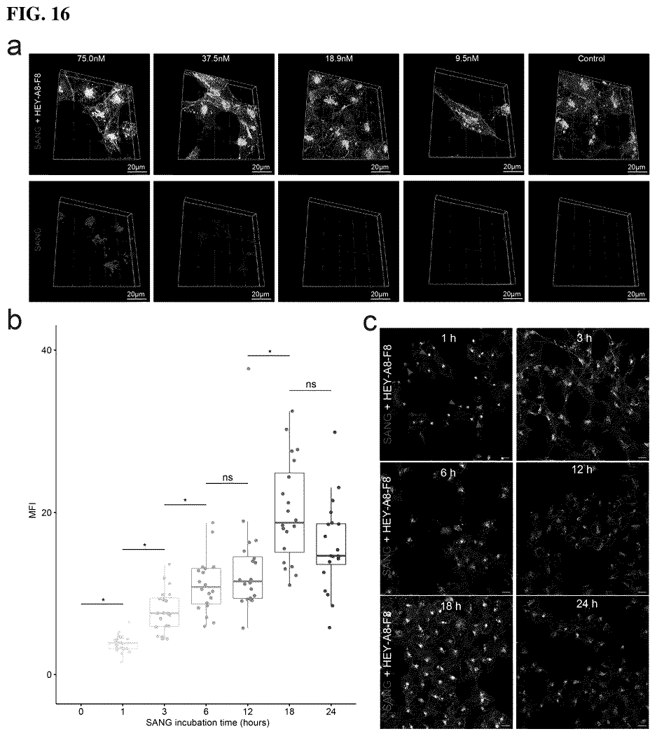

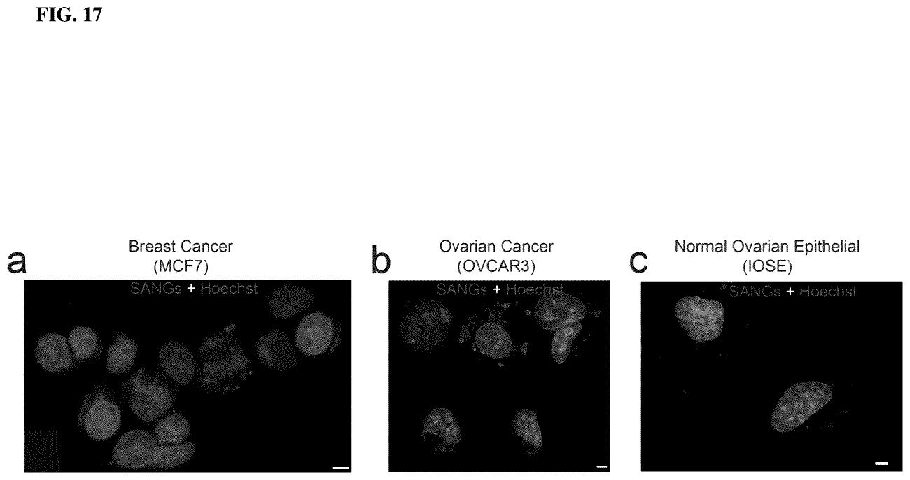

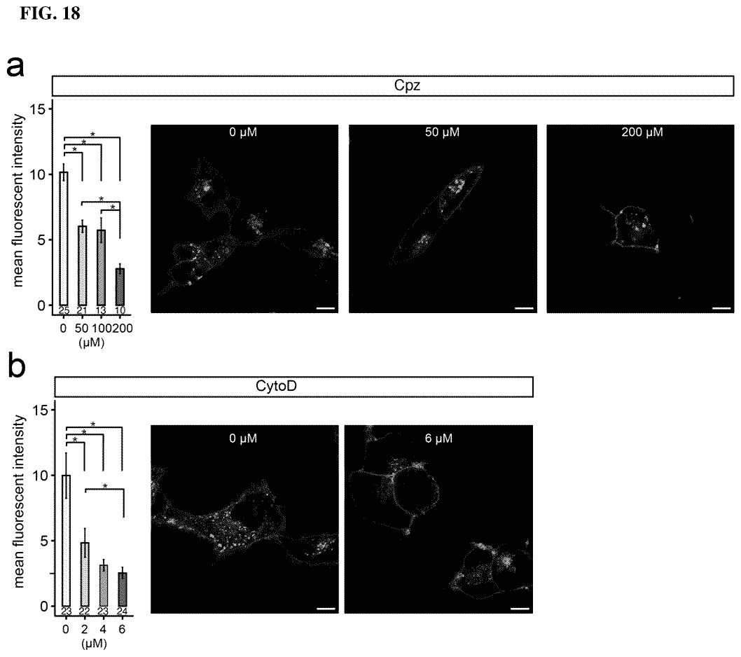

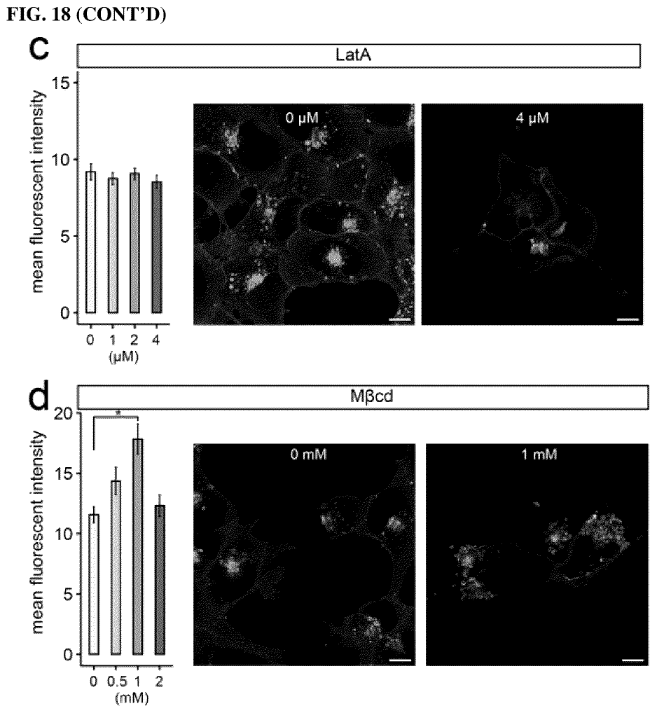

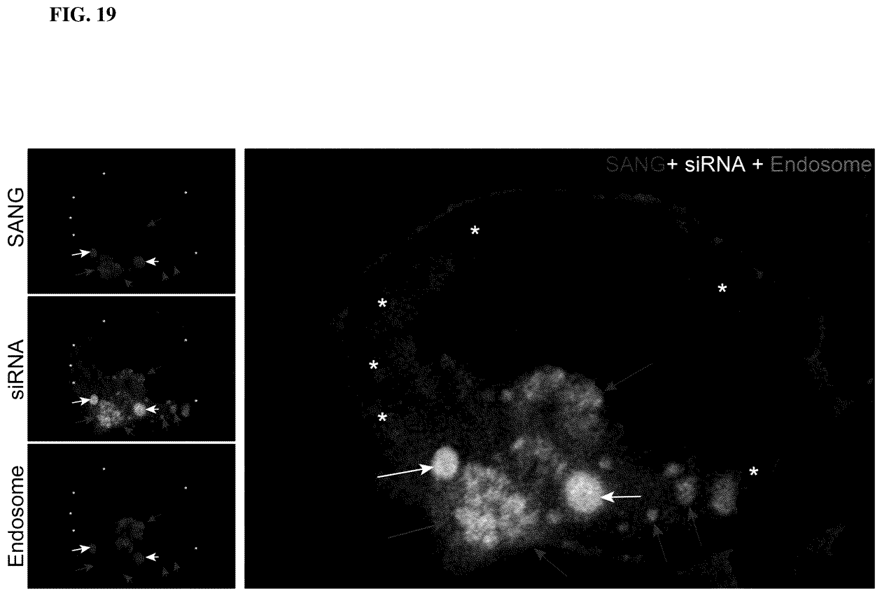

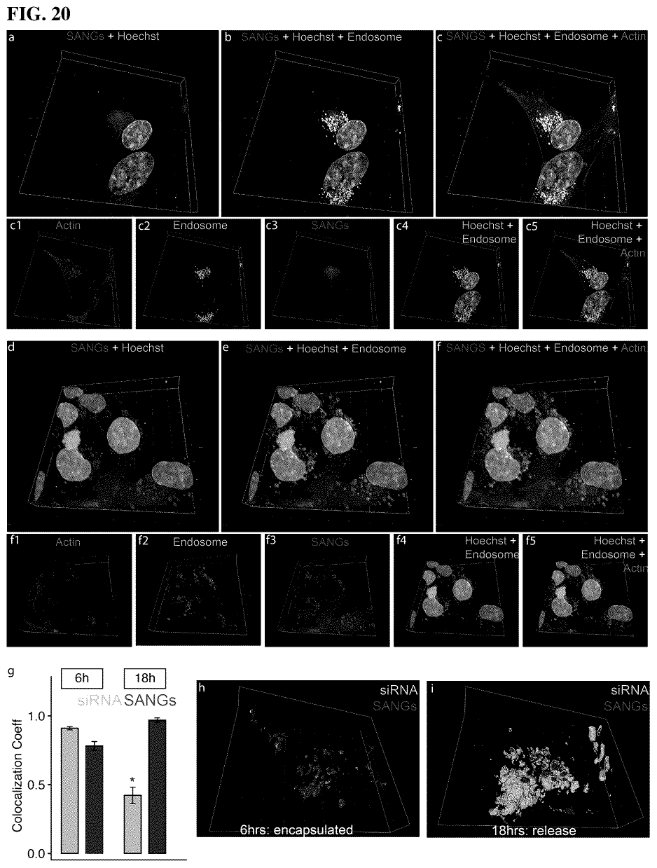

SANGs were taken up readily by HEY-A8-F8 cells in dose- and time-dependent fashion (FIG. 8, panels g-h and FIG. 16). Application of SANGs for increasing lengths of time (ranging from 30 minutes to 24 hours) revealed detectable intracellular uptake by 1 hour, which increased until maximal fluorescent signals were reached at 18 hours (FIG. 16). Internalization was comparable for epithelial (OVCAR3) and mesenchymal (HEY-A8-F8) type ovarian cancer cells, as well as MCF7 breast cancer cells (FIG. 17). Internalized SANGs remained unchanged in terms of cellular localization and fluorescence intensity for at least 24 hours (FIGS. 16-17). SANG cellular uptake was shown to be energy-, clathrin-, and macropinocytotic-dependent (FIG. 9, panels a-c, FIG. 18) and unperturbed by inhibition of caveolar mechanisms (FIG. 18), suggesting a combination of clathrin-mediated endosomal and macropinocytotic pathways. This conclusion was further corroborated by live-cell imaging with an endosomal marker (wheat germ agglutinin, WGA)(26, 27)) and SANGs containing dye-labelled siRNA, showing simultaneous co-localization of all components in HEY-A8-F8 cells at early time points up to six hours (FIG. 9, panel h, FIG. 19). After 18 hours, however, siRNA and SANGs were observed to be distributed throughout the cytoplasm (FIG. 9, panels d-f, FIG. 20), with low correlation (R=0.142±0.16, n=24) between siRNA and endosomes, suggesting an efficient process of endosomal escape and release from the particles (FIG. 9, panel g).

Blockade of epidermal growth factor receptor (EGFR) kinase activity has been used for cancer therapy. However, other kinase-independent functions of EGFR also mediate pro-survival and chemo-resistance, so siRNA mediated knockdown should be able to achieve levels of therapeutic efficacy inaccessible to kinase inhibition alone. Consistent with the apparent escape of siRNA to the cytosol, efficient suppression of EGFR mRNA and proteins was observed in overexpressing HEY-A8-F8 cells after treatment with SANGs formulated with siRNA against EGFR (20 nM) (FIG. 21). The same formulation substituting the appropriate siRNA induced similar knockdown of KRAS, Glut1, and miRNA-429, all known regulators of cancer cell proliferation, migration, and invasion. SANGs containing scrambled siRNA had no effect (FIG. 21).

### SANG Biodistribution and Retention in Mouse and Rat

The in vivo biodistribution of SANGs was explored with xenograft and orthotopic murine models of ovarian and breast cancer and a genetically-engineered rat model of colorectal cancer to survey different tumor types, induction methods, and species, all in the context of a functioning immune system. Each of the cancer strains employed were engineered to express luciferase and thus could be readily followed by bioluminescence imaging (BLI).

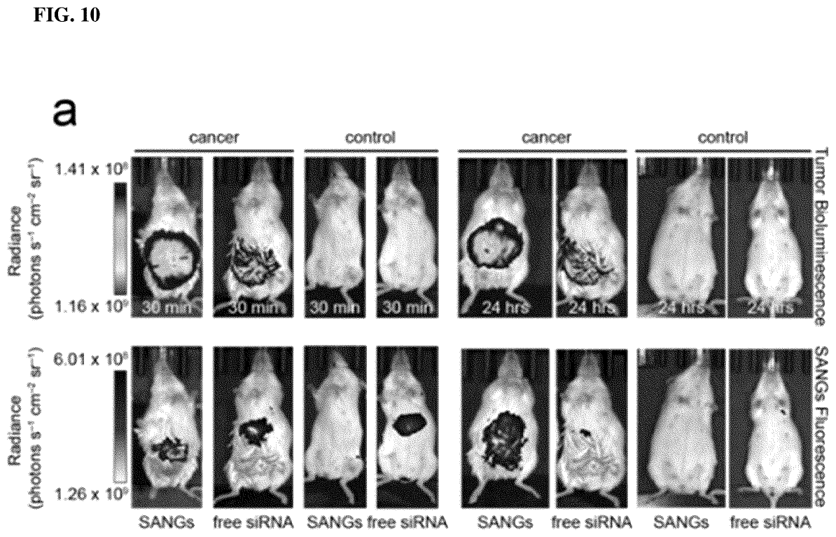

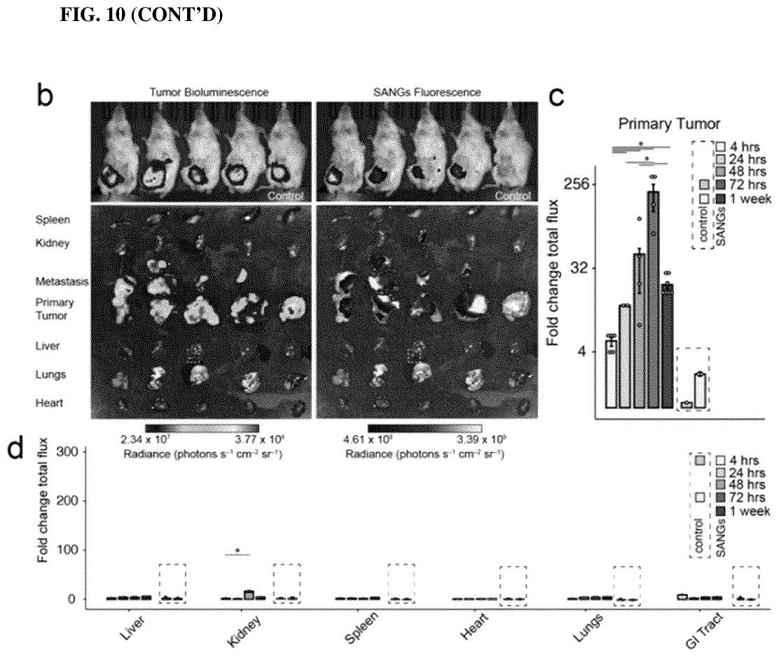

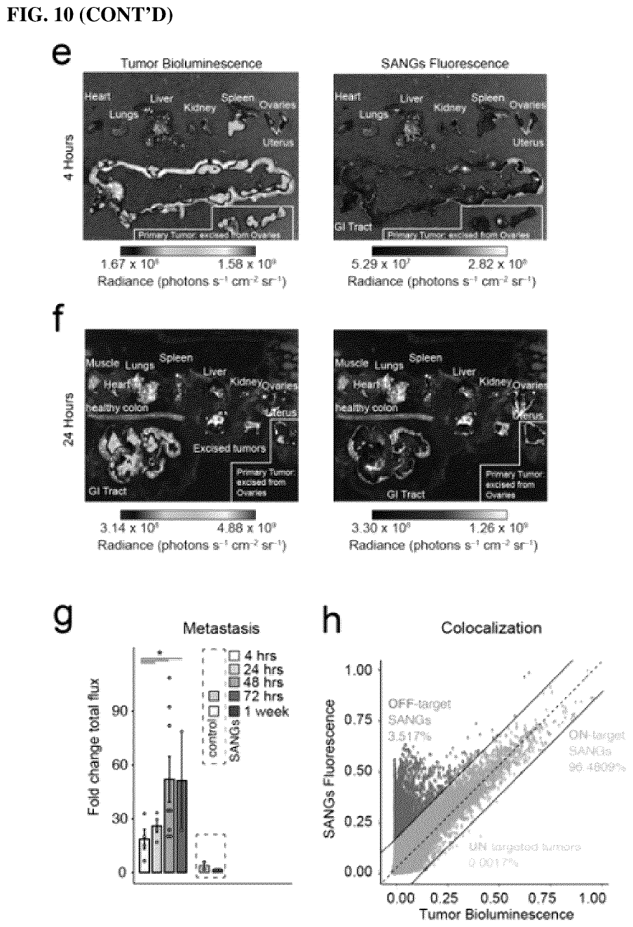

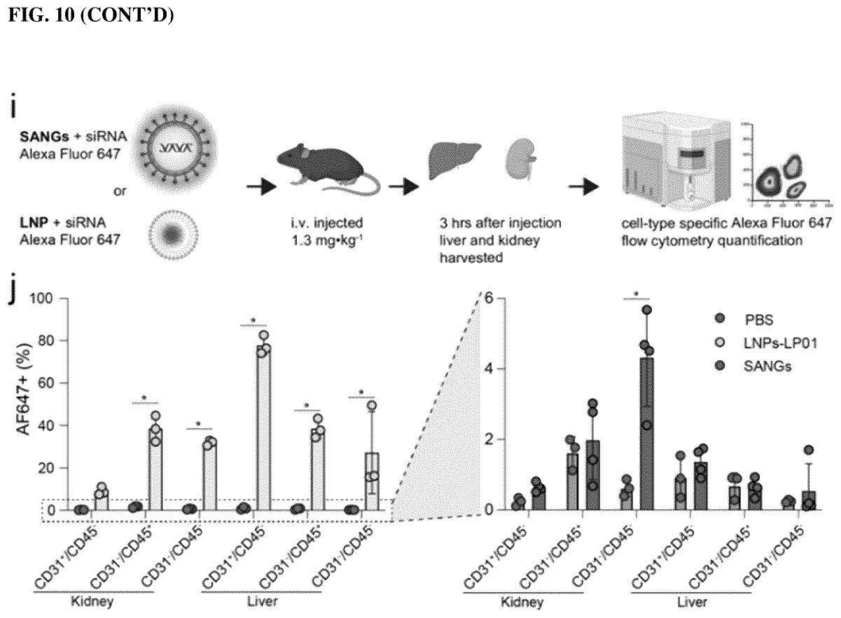

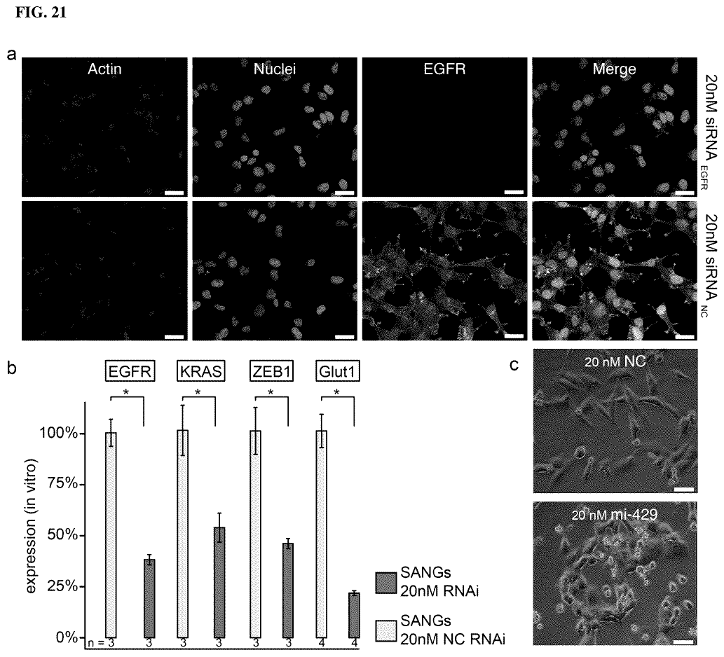

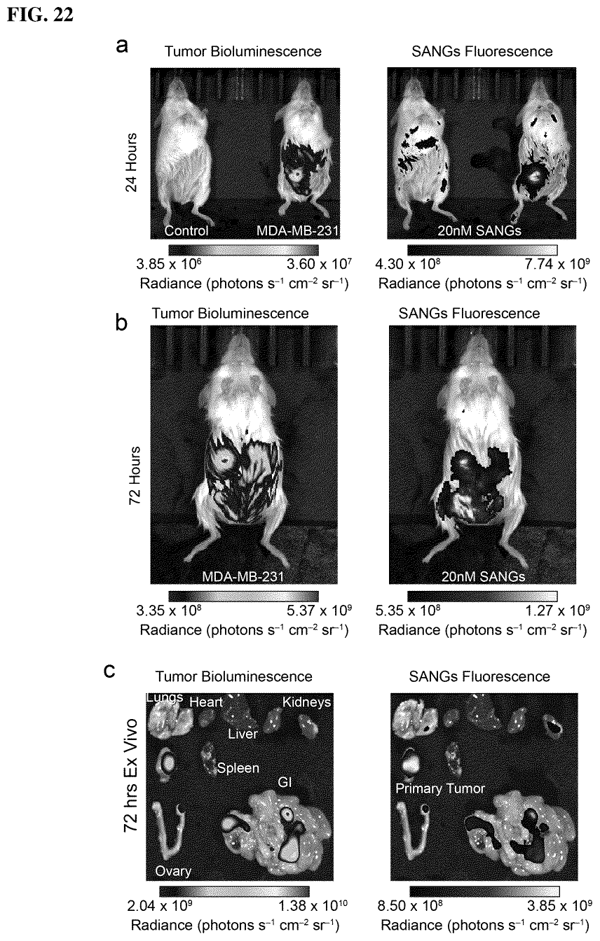

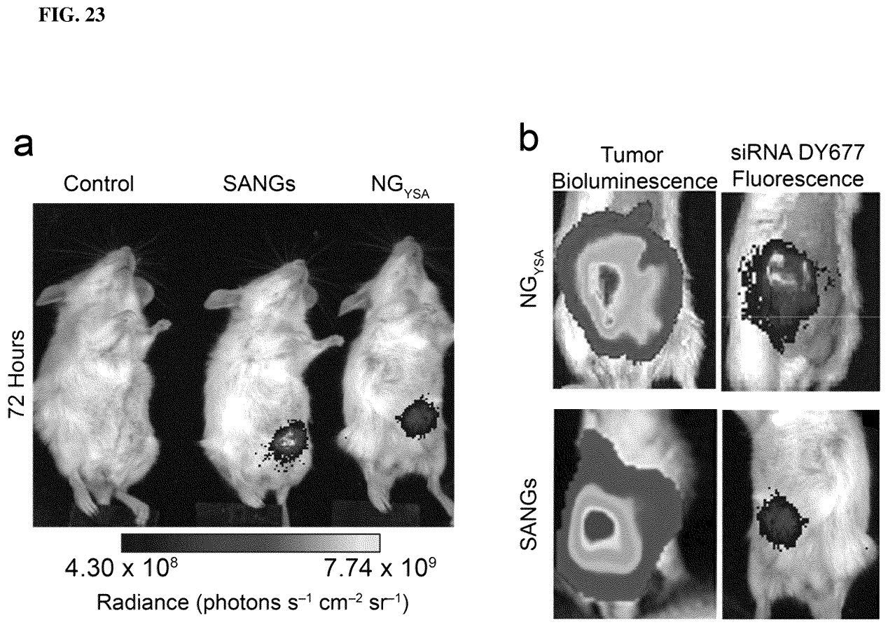

Within 30 minutes following i.v. injection in murine ovarian and breast cancer models, SANGs accumulated in regions with higher BLI signal indicating presumptive tumoral targeting (FIG. 10, panel a and FIG. 22). In vivo distribution of SANGs was distinct from unloaded siRNA and free dyes, which were detected in regions devoid of BLI signal in lungs (within 5 min) and in liver shortly thereafter (30 min) (FIG. 10, panel a). By 24 h, the specificity of biodistribution improved out to 72 h (FIG. 10, panel b) where little (if any) SANGs were detected outside BLI inferred tumors. The in vivo biodistribution and siRNA delivery of SANGs was equivalent to that previously observed for targeted (YSA-functionalized) nanohydrogels (as previously described(25) and repeated in FIG. 23 indicating that the targeting ligand is irrelevant). SANGs remained closely associated with BLI signals 7 days after i.v. delivery indicating preferential retention in tumors.

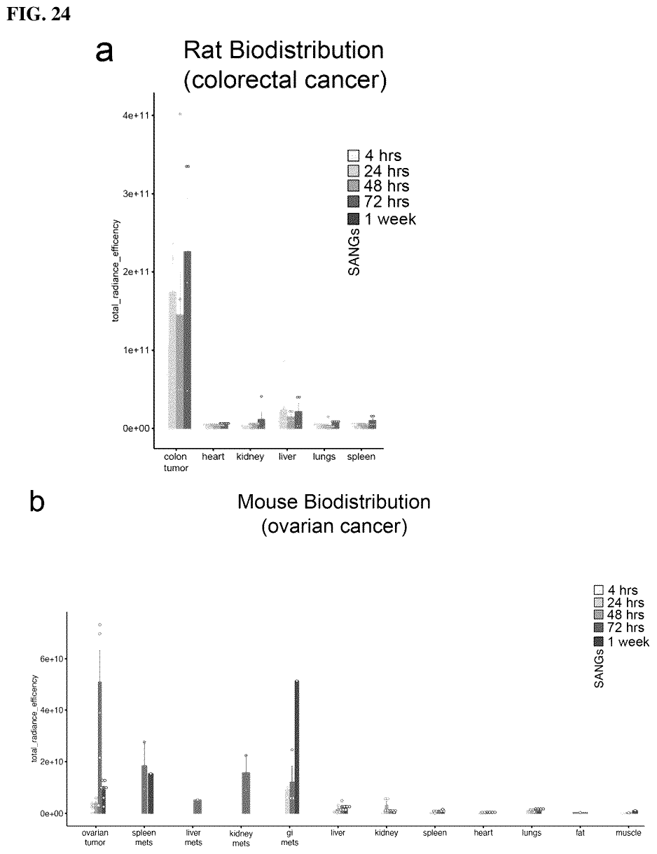

Analysis of excised tissues confirmed SANGs rapidly targeted tumors, reaching 5.1±1.2-fold increase by 4 hours and a maximum (approximately 200-fold above background) at 72 hours (FIG. 10, panel c and FIG. 24). While significantly reduced compared to peak, it was found that SANGs were retained in primary tumors 7 days after systemic delivery (FIG. 10, panel c). In contrast, intravenous infusion of free siRNA (20 μM) gave significantly lower tumor localization at 3 and 7 days (1.14±0.02 and 2.29±0.17 fold above background, FIG. 10, panel c)(28). Quantitative analysis of SANG biodistribution to major organs systems confirmed that non-cancerous organs remained largely devoid of SANG signal out to one week with the exception of a transient increase detected in kidney (FIG. 10, panel d and FIG. 24, panel b).

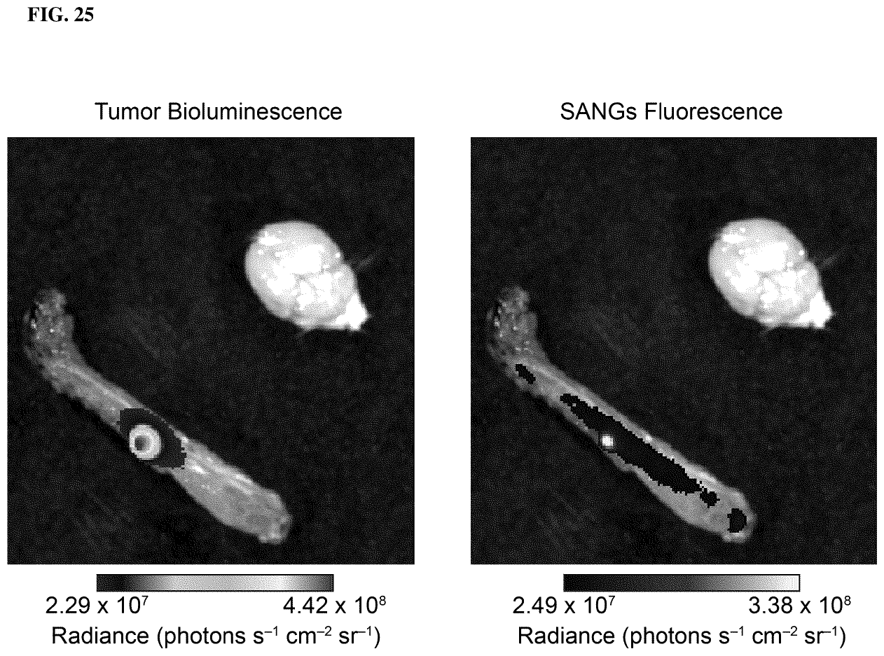

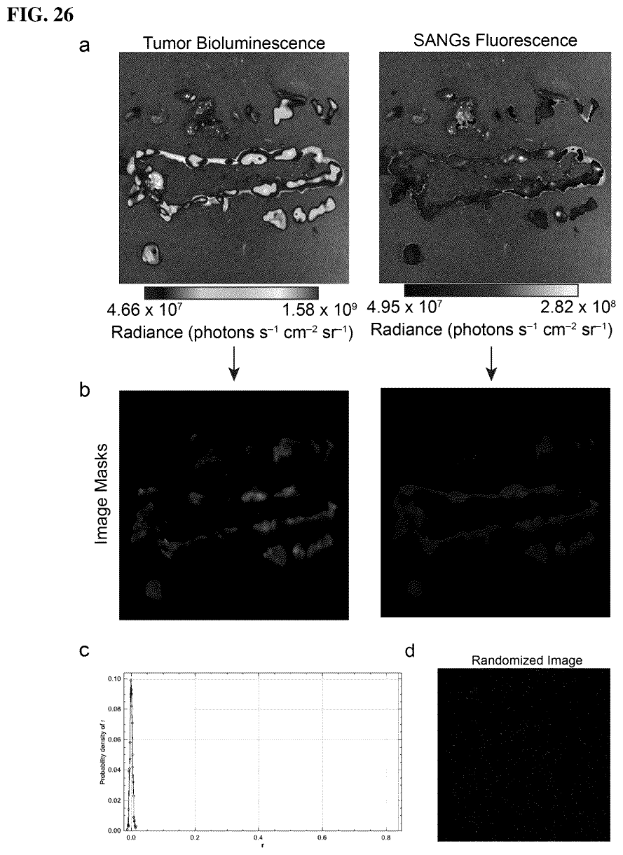

To test the metastatic targeting capacity of SANGs, a limitation of existing RNAi delivery platforms(29), late-stage ovarian cancer by IP administration of HEY-A8-F8 cells was modeled. After confirming tumor induction and extensive abdominal metastasis, SANGs were administered intravenously and quantified biodistribution as described above. Expansive tumor load was observed across the spleen, GI-tract, liver (FIG. 10, panels e-f), and occasionally epidural metastasis (FIG. 25). Surprisingly, rapid (4 h) and highly specific accumulation of SANGs in diffuse metastatic lesions (FIG. 10, panel e) was also observed. The temporal biodistribution in metastatic loci closely mirrored that of primary tumors independent of organ (FIG. 10, panel g) while signal in the non-cancerous portions of organs remained largely devoid of SANG signal (FIG. 10, panels d-f and FIGS. 24, 26).

Co-localization analysis of ex vivo SANG fluorescence and tumor bioluminescence (FIG. 10, panel h) showed strong association between SANGs and diffuse metastases (R=0.84, p(25)0.001, FIG. 26) with >96% of the SANG signal residing within 15% of the line of identity. Few if any (<0.1%) metastatic loci were untargeted by SANGs (below line of identity) while only <3.5% of SANGs were detected in regions without any BLI confirmed metastasis. These results indicate a low level of off-target accumulation and corroborate biodistribution data observed in less metastatic murine models. Taken together, these results indicate that SANGs rapidly target both primary and metastatic murine tumors, are retained for extended periods of time, and have minimal off-target accumulation (FIG. 10, panel d).

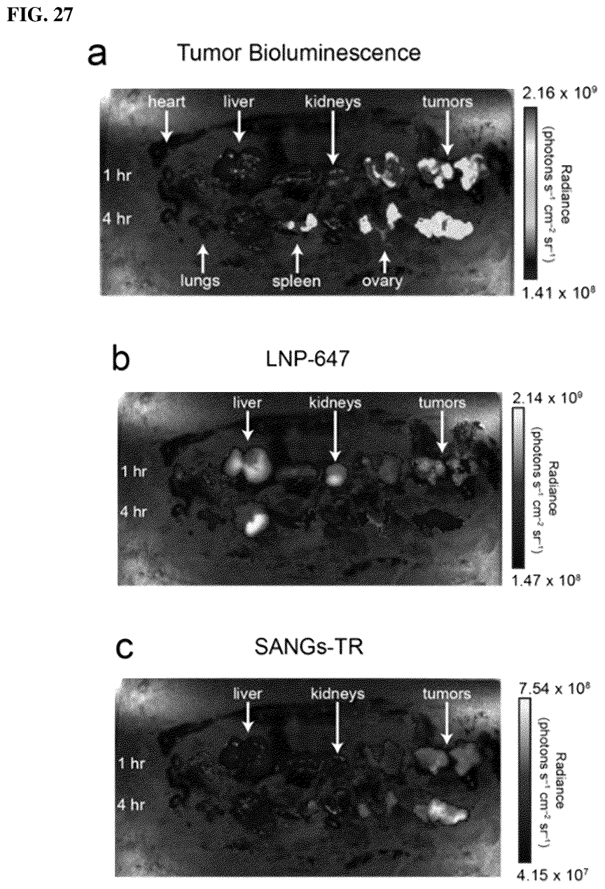

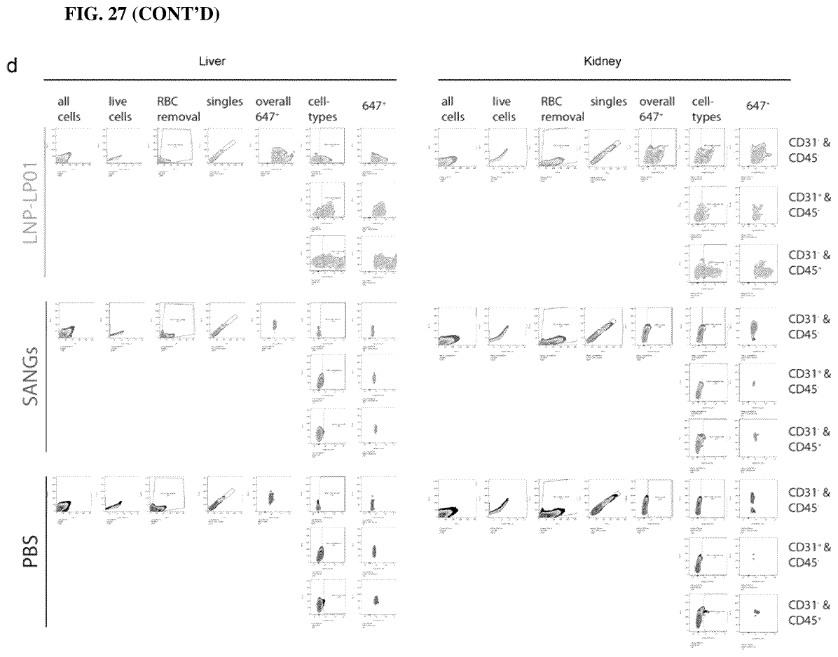

To quantitatively assess SANGs apparent avoidance of liver and kidney entrainment, the cellular distribution of SANGs was compared to that of a well-documented liver-targeting LNP formulation based on the lipid LP01 (FIG. 10, panel i) (30). In separate cohorts of control mice, SANGs or LNP-LP01 (n=4 and n=3 independent biological replicates respectively) were intravenously injected, each formulated to carry AlexaFluor-647 labeled siRNA against EGFR. After three hours, CD31⁻CD45⁻, CD31⁺CD45⁻, and CD31⁻CD45⁺ cell populations were isolated from liver and kidney and the number of those cells carrying the AlexaFluor dye was quantitatively determined. As expected, significantly more cells extracted from liver in LNP-LP01 treated animals were dye labeled compared to PBS controls (FIG. 10, panel j). With the exception of presumptive renal endothelial cells, significantly more CD31⁻/CD45⁺ and CD31⁻/CD45⁻ cells extracted from kidneys also were labeled with the dye, presumably by LNP uptake (FIG. 10, panel h). In contrast, it was found that only 4% of CD31⁻/CD45⁻ renal cells from SANGs treated animals to contain significant amounts of dye relative to PBS treated controls (FIG. 10, panel j). Collectively, across all cell-types studied, 10-30 times fewer dye-labeled cells in SANGs treated animals were found compared to LNPs (FIG. 27), thereby verifying whole organ and confocal microscopy biodistribution data (FIG. 27). Ovarian tumor bearing mice were intravenously injected simultaneously with LNP-LP01 formulated to carry AlexaFluor-647 labeled siRNA against EGFR and SANGs formulated to carry Texas-Red labeled siRNA against EGFR. Analysis of excised tissues at one and four hours after injection demonstrates orthogonal biodistribution between SANGs and LNP-LP01 (FIG. 27). While the use of different fluorophores precludes quantitative comparisons, these data qualitatively showcase the ability of SANGs to target solid tumors compared to a state-of-the-art delivery platform.

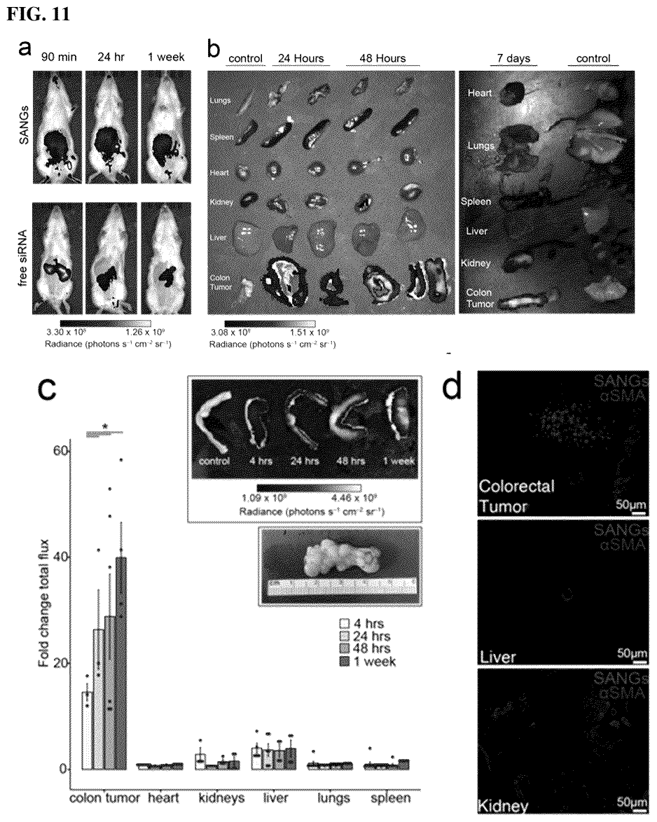

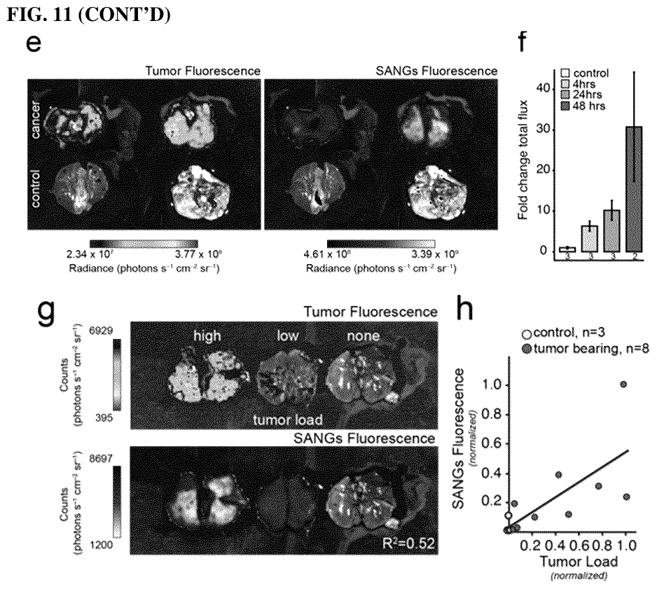

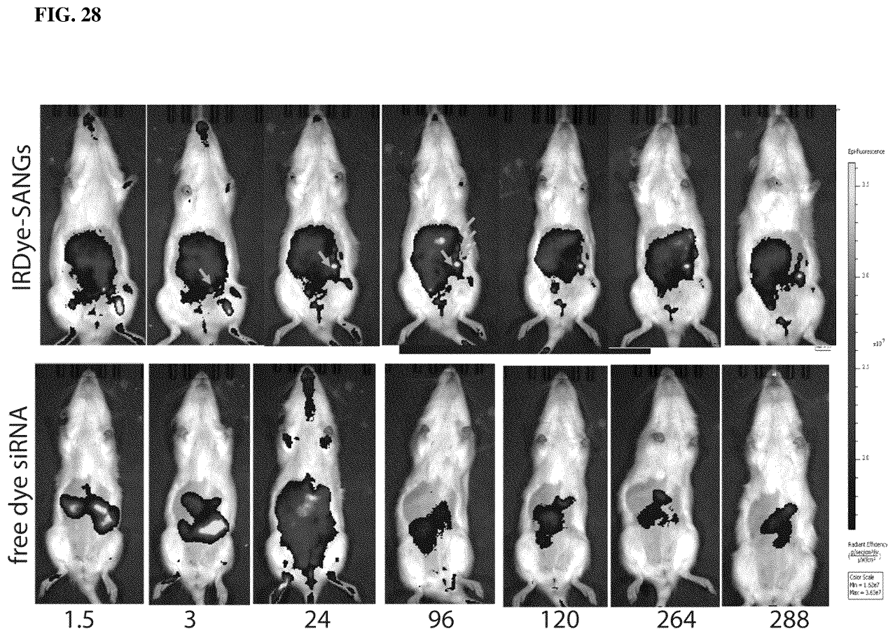

Intravenous delivery of SANGs to rats genetically engineered to spontaneously develop colorectal cancer resulted in immediate diffuse SANG signal across the abdominal cavity which began to coalesce in small punctate intensities around the perimeter of the abdomen, suggestive of preferential targeting to the diffuse tumors (FIG. 11, panel a and FIG. 28). SANG distribution remained localized in this fashion for 7-days with increasing signal-to-noise ratio over time, indicating preferential tumor retention. In contrast, free siRNA distributed mostly to the liver followed by rapid elimination (FIG. 11, panel a). Ex vivo analysis of colon with diffuse adenocarcinomas (FIG. 11, panel b) showed detectable SANG signal at 4 hours, increased signal at 24 and 48 hours, and a maximal recorded value at 7 days (FIG. 11c); mouse biodistribution peaked at 72 h (FIG. 10, panel c). As in mouse, SANGs remained largely absent from other major rat organs with the exception of a nominal increase detected in liver and in the kidney (FIG. 11, panel c) which was confirmed by confocal microscopy (FIG. 11, panel d). Intravenous delivery of SANGs (1 mg·kg⁻¹) to lung tumor- (GFP-MDA-MB-231) and non-tumor-bearing SRG rats showed significantly higher SANG delivery to tumor bearing animals (FIG. 11, panel e). SANGs tumor biodistribution progressively increase over time (FIG. 11, panel f), consistent with in vivo behavior in mice with ovarian cancer and rats with colorectal cancer and correlated with tumor fluorescent intensity, a proxy for tumor load (FIG. 11, panels g-h).

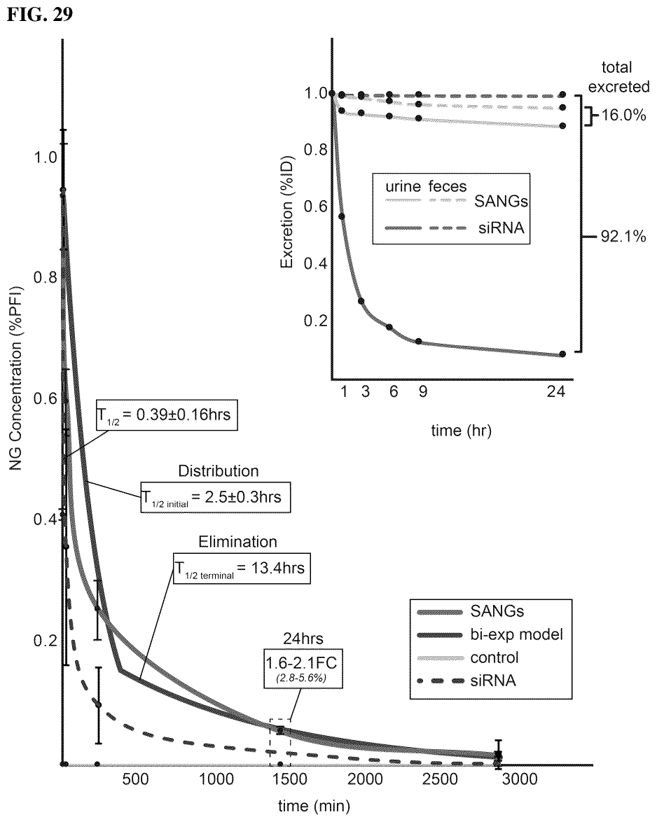

In vivo pharmacokinetics and clearance studies revealed an apparent bi-exponential blood distribution half-life (a) of approximately 2.5 h for SANGs and an elimination half-life (β) of ~13.4 h (FIG. 29) in rat. While this value is comparable to that of other nanoparticle formulations(28, 31), SANGs behave differently in at least one important respect. Relatively high SANG concentrations were found in blood at 24 and 48 h with between 2.8-5.6% of the initial dose remaining in circulation, a factor that likely played a role in the favorable tumor biodistribution and retention. Analysis of urine and feces showed rapid and near complete (~92%) excretion of naked siRNA by 24 h which was dominated by renal metabolism whereas only 16% of the SANG dose was excreted during the first 24 h. These data show that a large fraction of SANGs leaves systemic circulation rapidly, partitions efficiently to tumors, and perhaps is also sequestered in low concentrations in tissue such as fat, to be released over time.

### In Vivo Functionality of SANGs

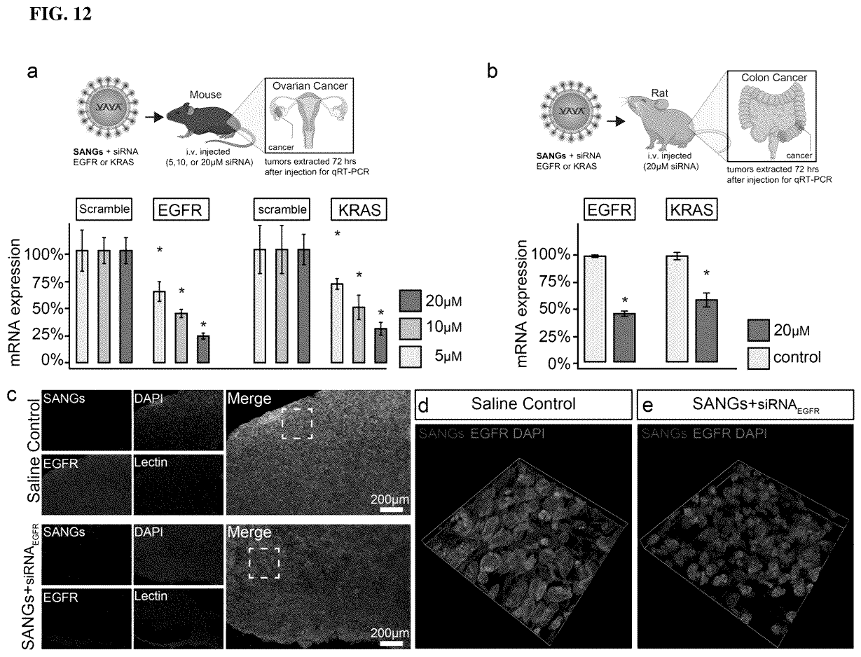

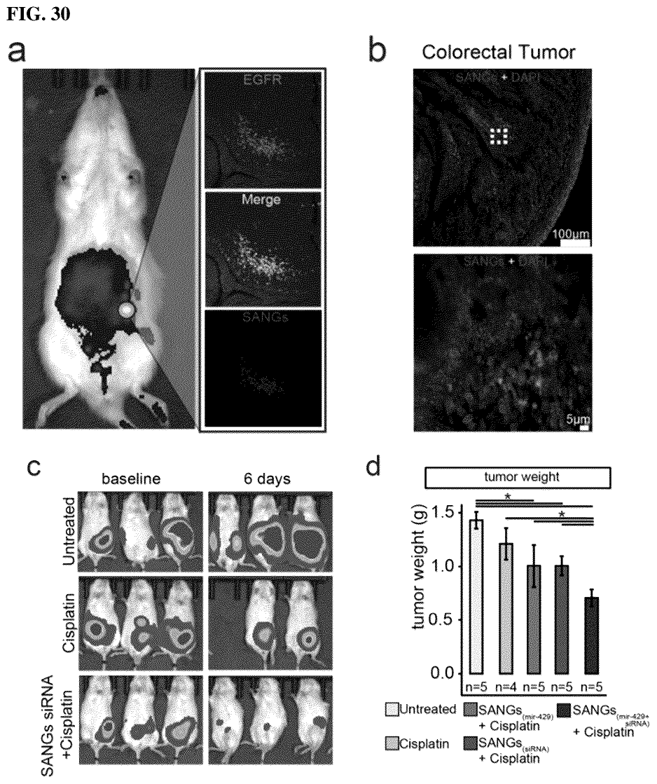

When examined by confocal fluorescence microscopy, SANGs were found distributed throughout ovarian carcinoma (HEY-A8-F8) tumor sections and deep within tumor parenchyma, exhibiting an extravasated distribution into the tumor interstitium (FIG. 12, panel a and FIG. 30). A similar result was observed in mice with orthotopically implanted ovarian carcinomas (HEY-A8-F8) and breast cancers (MDA-MB-231) (FIG. 24) as well as in rats with advanced colorectal cancer, where strong co-localization with EGFR-overexpressing tumor cells (FIG. 30) was seen. In all cases, the extravasated distribution was retained for at least 7 days.

Across murine ovarian (HEY-A8-F8) and rat colorectal cancers, it was found that single intravenous infusion of SANGs loaded with siRNA against EGFR or KRAS resulted in dose-dependent reduction in mRNA (FIG. 12, panels a-b) and protein expression (FIG. 12, panels c-e) in tumor tissue. Therapeutic efficacy of SANG-siRNA was then tested in mice with tumors established from HEY-A8-F8 ovarian carcinoma cells. Enhanced expression of EGFR by these cells is correlated with drug resistance, and is a well-established model(32, 33). Groups of mice were treated with two independent doses of SANGs, 24 hours apart, the first loaded with mir-429 and the second with siRNA against EGFR, because mir-429 driven EMT reversal requires time to induce the necessary phenotypic change. Intraperitoneal cisplatin was then delivered 24 hours later. Cisplatin alone was used as standard of care control. Six days after treatment, dramatic improvements relative to drug alone were evident from significant inhibition of tumor growth and reduction in tumor weight (FIG. 30). These data indicate that SANGs successfully extravasate, penetrate tumor microenvironments, gain access to the specific cells of interest in a species- and tumor-agnostic manner, and sensitize previously resistant tumors in vivo.

### Systemic Delivery of SANGs is Minimally Toxic

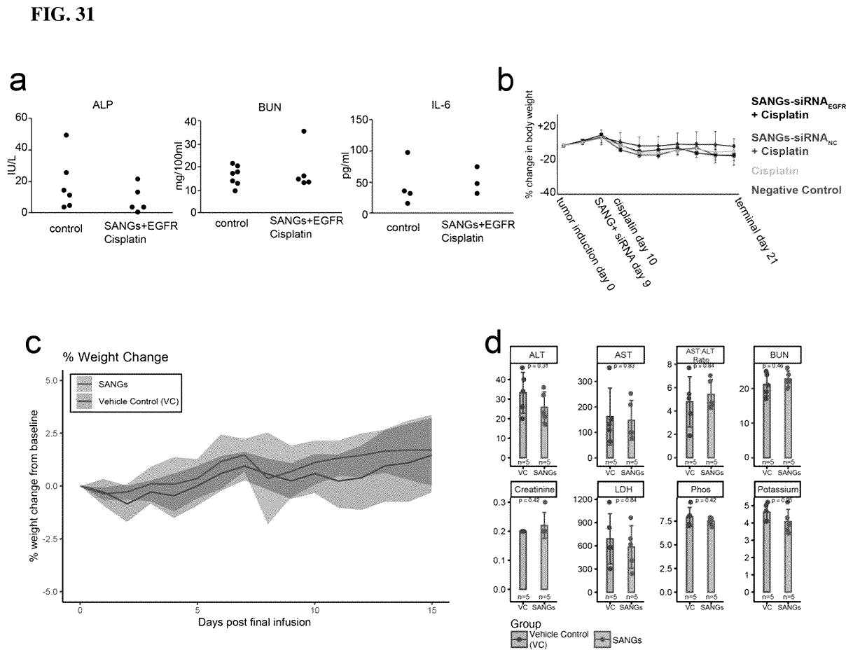

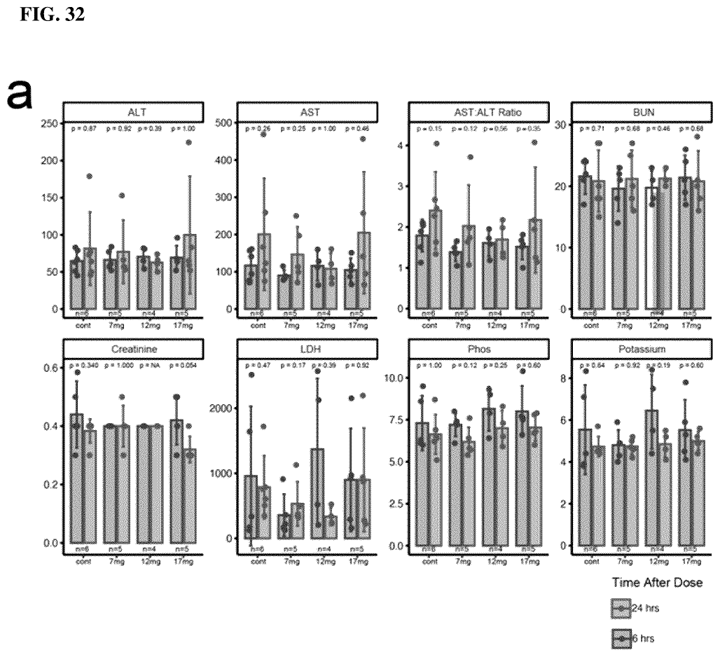

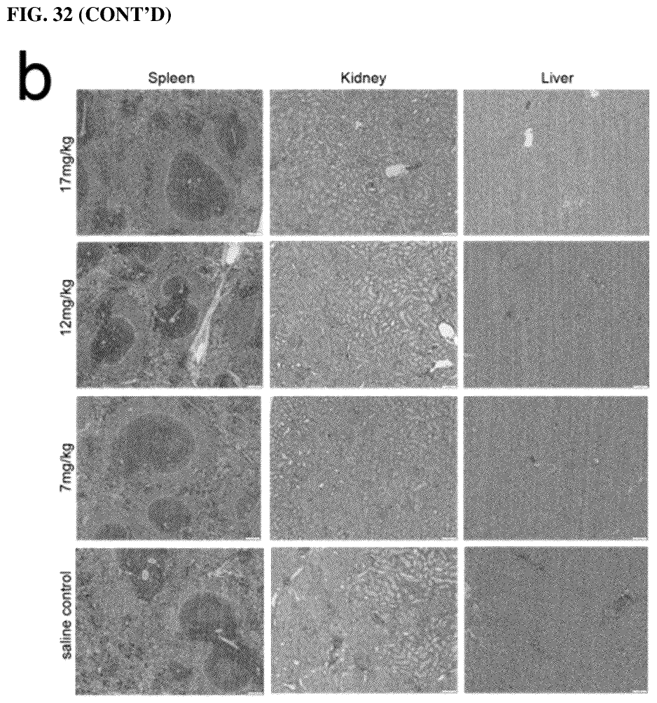

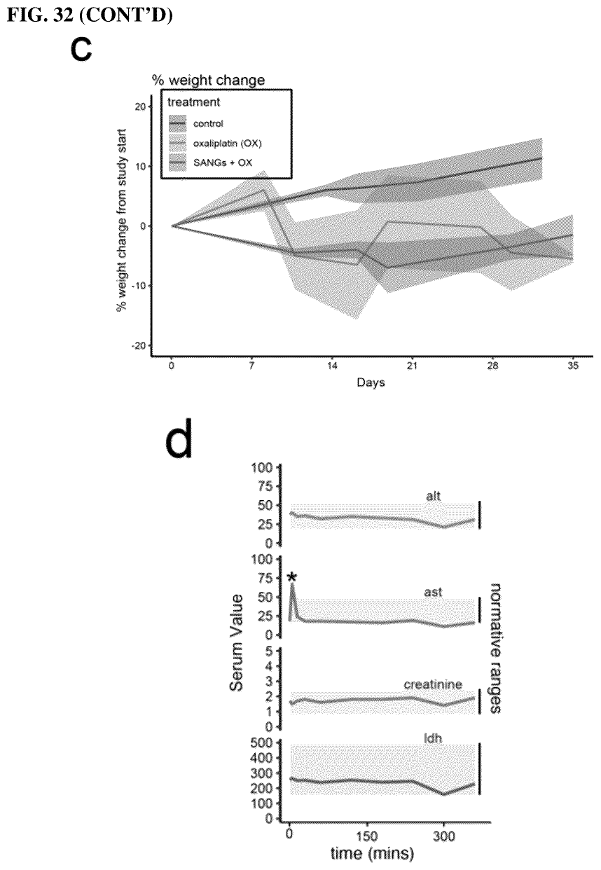

A variety of tests revealed no significant toxicity associated with high doses of either empty or scrambled miRNA/siRNA-loaded SANGs. These included observations with outbred mice (FIG. 31) and NOD-SCID mice with ovarian tumors including delivery of cisplatin (FIG. 31). A repeat-dose tolerability study of high-dose SANG-siRNA and co-infused oxaliplatin in rats with advanced colorectal cancer(34) produced no mortality or any clinical signs of distress while weights recovered back to baseline by study completion (FIG. 32). A maximal tolerable dose study in rats revealed only minor changes in blood chemistry at 6 and 24 hours (FIG. 32) and no difference from controls in histopathology of major organs for the three escalating doses (FIG. 32). Finally, a single-acute dose study in an adult female (73 kg) Yucatan swine showed no clinically meaningful deviances out to 6 hours following i.v. infusion of SANGs-siRNA at the same high dose (7 mg·kg⁻¹) with the exception of a transient increase in AST at 5 minutes (FIG. 32). Thus, SANGs induce minimal, if any, toxicity in a variety of animals and experimental conditions. Taken together, these data establish a wide preliminary safety profile and strongly support the systemic use of SANG to deliver RNAi to solid tumor cancers.

### Cancer Specific Delivery Achieved Through Emergent Self-Agglomerating Mechanism

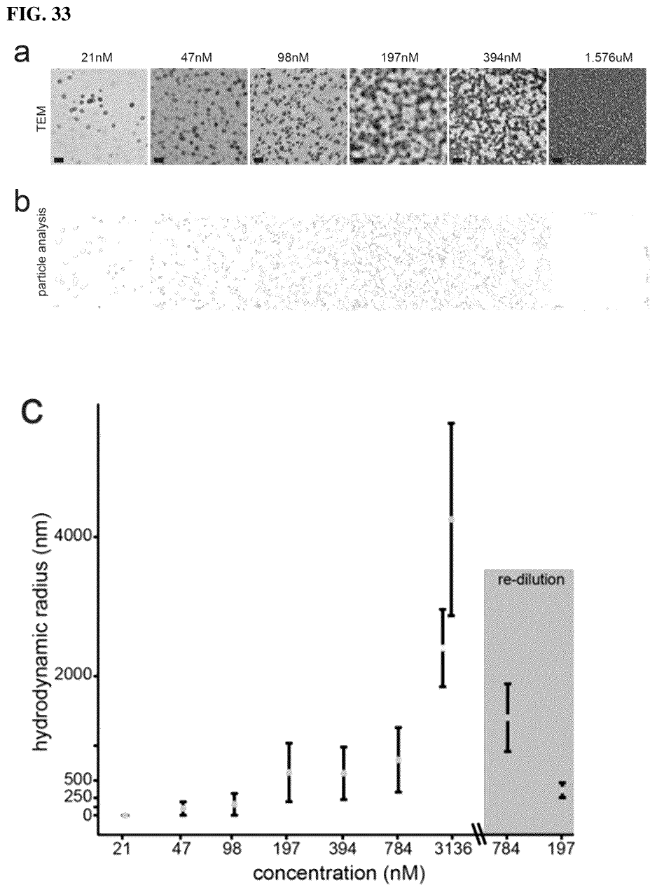

The results described here set SANGs apart from most nanoparticulate delivery vehicles, their most striking property being selective homing to tumors without the use of designed ligands for cell-surface markers. Five complimentary methods were used to probe the mechanism(s) responsible for this in vivo performance. It was sought to distinguish between mechanisms involving cell binding vs. physical properties of the particles that may promote selective distribution to tumor tissue. First, TEM was used to measure the size and number of SANG particles over concentrations ranging from 25.5 nM to 1.6 μM (FIG. 13, panels a,b). Results revealed an interesting dynamic behavior. At the lowest three concentrations studied, the number and size distribution of SANGs closely matched the values predicted by a linear dependence of concentration on the number of particles, expected for any solute that does not undergo changes in form with changes in concentration (FIG. 13, panel b). At higher SANG concentrations, however, this relationship no longer held. Instead, progressively smaller numbers of SANG particles were observed, with increasing sizes and size distributions (FIG. 13, panel b and FIG. 33). High magnification views showed different representative forms of tightly clustered SANGs that emerged above 156 nM. Similarly, aqueous dynamic light scattering (DLS) on two different instruments showed strongly monodispersed hydrodynamic size distributions at low concentration (52 nM), but clear evidence of larger and less regular aggregates at 156 nM (FIG. 13, panel c and FIG. 33). When concentrated SANGs samples were re-diluted, much of the material returned to the original size range on average as indicated with DLS (FIG. 13, panel a and FIG. 33) while larger aggregated states were detected with TEM (FIG. 13, panel a).

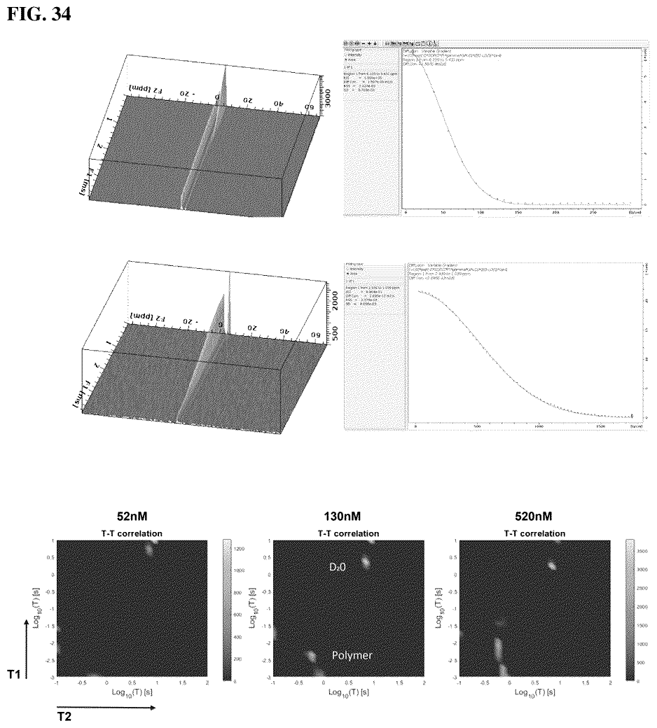

Parallel diffusion-ordered NMR measurements allowed us to detect diffusional changes caused by aggregation. A significant decrease in diffusion coefficient (21.5%) and T2 relaxation time was observed over a 10-fold range of SANG concentration (FIG. 13, panel d and FIG. 34). The former parameter indicates slower particle movement in solution and the latter reflects restricted molecular motion(35, 36), both consistent with nanogel agglomeration in solution.

Liquid-phase transmission electron microscopy (LP-TEM) enabled us to directly measure SANG behavior with nanometer-scale and sub-second resolution in a liquid environment. Liquid cell chambers with 150 nm aperture were used to provide adequate room for particle motion. SANGs were readily detected above background and demonstrated clear evidence of nanogel agglomeration throughout every field of view studied (FIG. 13, panel e). SANGs were observed to ‘snap’ together in long chains and islands, similar to structures observed with negative stain TEM (FIG. 13, panel a). Critically, these aggregates retained their close association as they rotated across fields of view (FIG. 13, panel e). This real-time, nanoscale evidence of nanogel agglomeration in naturalistic conditions provides the first direct observations of the emergent SANG materials property and supports our underlying hypothesis.

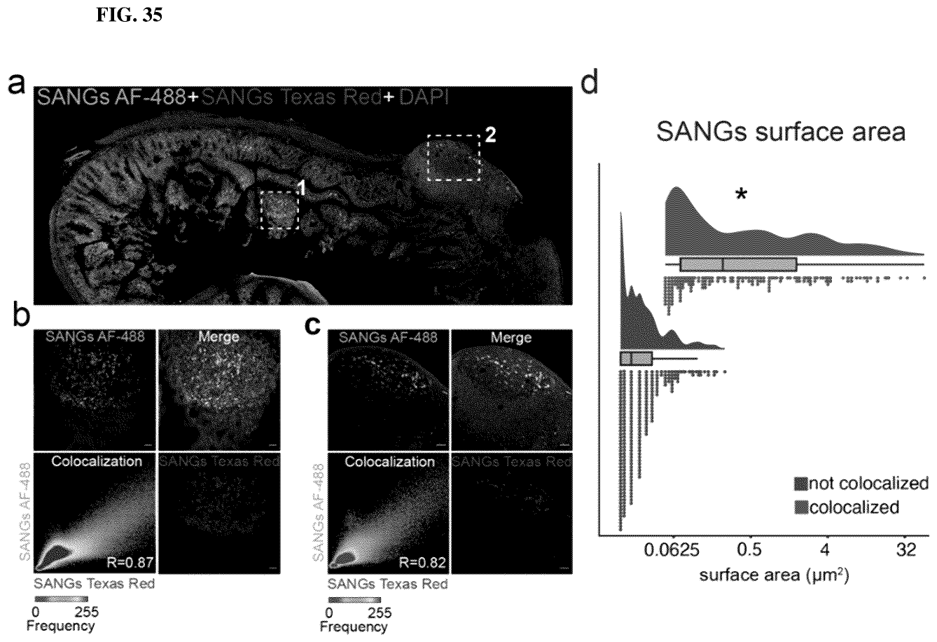

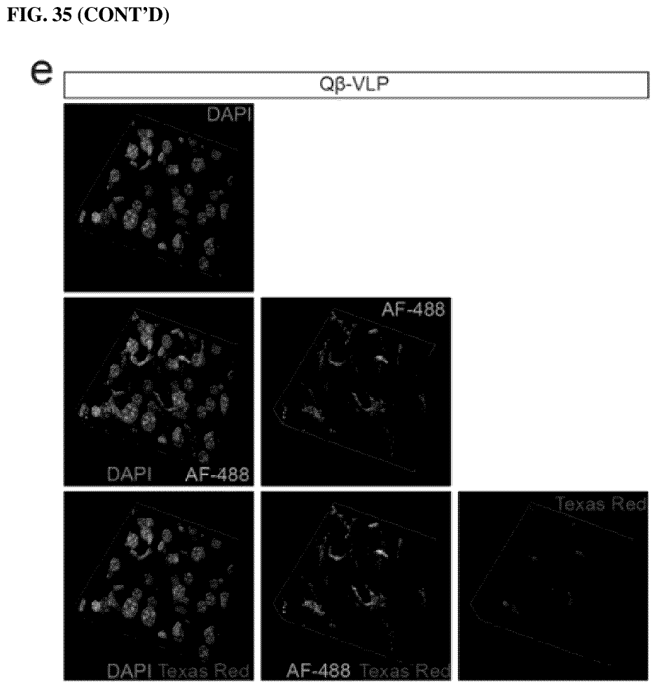

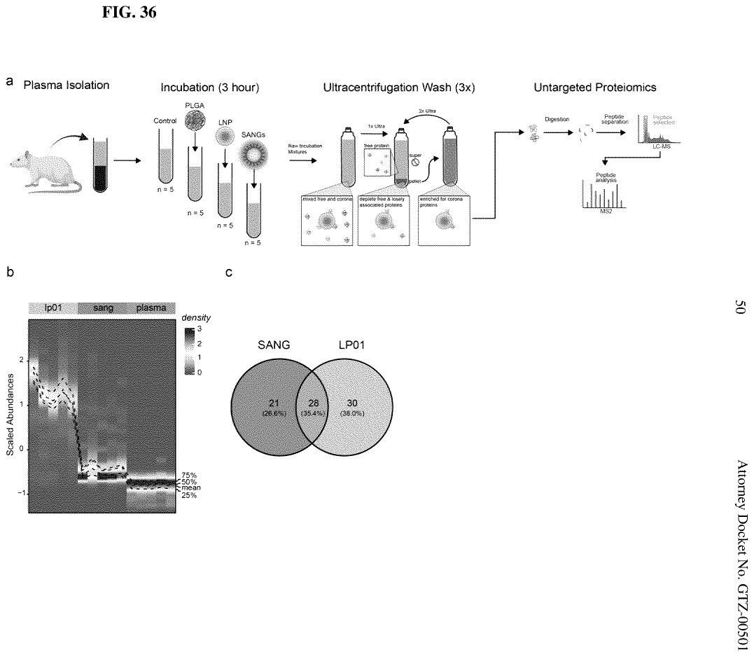

To test if nanogel aggregation occurs in vivo, two populations of SANGs conjugated to different fluorophores were sequentially intravenously infused in rats with advanced colorectal cancer. If SANGs do not agglomerate in vivo, fluorescent signals from both populations would co-localize at a nominal frequency determined by chance. Co-localization to a significantly greater degree would indicate that agglomeration does indeed occur. Fluorescent signals from SANGS of both colors were readily detected (FIG. 13, panel f and FIG. 35) and found to be co-localized to a degree significantly greater than that expected by chance (Costes randomization-based co-localization R=0.85±0.02, p<0.001, n=3 animals, 3 sections each). Furthermore, deposits showing both colors were significantly larger (~74-fold) than SANGs of a single color (FIG. 35). A very stable, non-aggregating comparator, the Qβ virus-like particle (Qβ-VLP), was dye-labeled in the same manner and was found in its expected location (liver)(37) with random co-localization frequency (FIG. 13, panel g, Costes randomization-based co-localization R=051±0.03, p<0.001, n=3 animals, 3 sections each). Collectively, results from these five methodologically independent experiments are consistent with the hypothesis that concentration-dependent aggregation is at least partially responsible for the preferential tumor targeting property of SANGs (FIG. 13, panel h).

### Comparative Proteomic Profiling Reveals Unique SANGs Biomolecular Corona.

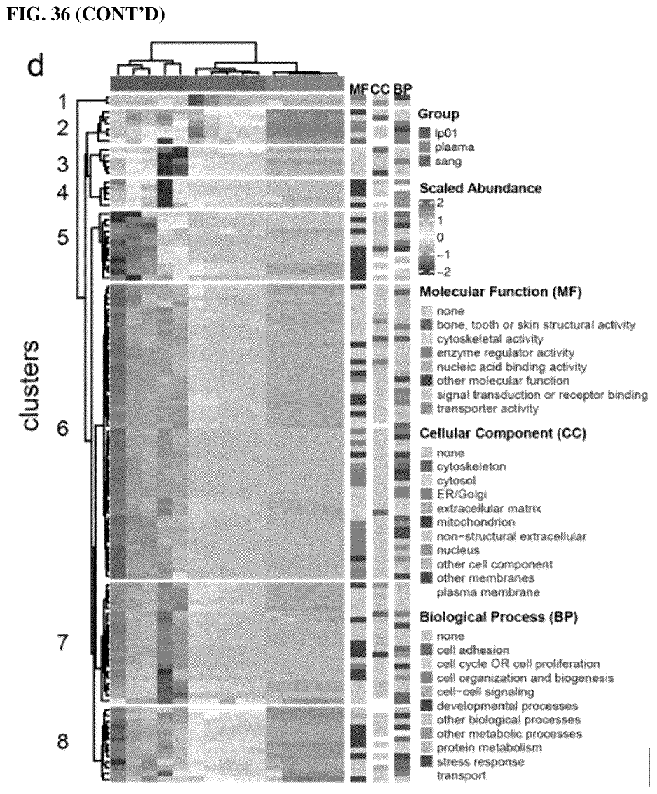

Given the role biomolecular coronas play in biodistribution, whether protein coronas form on SANGs was tested (FIG. 36, panel a). Quantitative proteomic study of the biomolecular corona of SANGs compared to coronas formed on LP01 nanoparticles, a commonly utilized drug delivery vehicle, revealed significant differences between platforms. LP-01 coronas contained significantly more and higher abundances of proteins than those associated with SANGs which was marginally higher than background-nonspecifically bound proteins (FIG. 36, panel b). FIG. 36, panel c illustrates the distribution of distinguishing corona proteins detected after filtering out background plasma proteins (FDR<0.001 and >1.5-fold-change relative to plasma sample). Protein identities included in Table 1.

TABLE 1
List of protein identities.

| | | | ““abundance | | | |
|---|---|---|---|---|---|---|
| “ | ““Accession”” | ““description”” | Ratio”” | ““group””” | | |
| “1 | ““P14046”” | ““Alpha-1-inhibitor 3”” | 14.547 | ““lp01_sig””” | | |
| “2 | ““P02680”” | ““Fibrinogen gamma chain”” | 9.513 | ““lp01_sig””” | | |
| “3 | ““A6J5V1”” | ““Fibrinogen | B beta | isoform CRA_b”” | 8.182 | ““lp01_sig””” |
| | | | polypeptide | | | |
| “4 | ““A0A0G2JYK0”” | ““Serine (or cysteine) | clade A | member 3N”” | 6.41 | ““lp01_sig””” |
| | | peptidase inhibitor | | | | |
| “5 | ““A6KFG4”” | ““Fibronectin”” | 5.105 | ““lp01_sig””” | | |
| “6 | ““A0A8I6AWQ8”” | ““Fetuin B”” | 5.023 | ““lp01_sig””” | | |
| “7 | ““A6HRN6”” | ““Alpha-1-B glycoprotein | isoform CRA_a”” | 4.628 | ““lp01_sig””” | |
| “8 | ““A0A0G2JU23”” | ““Apolipoprotein A-I”” | 2.891 | ““lp01_sig””” | | |
| “9 | ““A6J7X0”” | ““Protein AMBP”” | 2.822 | ““lp01_sig””” | | |
| “10 | ““F7EYF5”” | ““Inter alpha-trypsin inhibitor | heavy chain 4”” | 2.395 | ““lp01_sig””” | |
| “11 | ““P55314”” | ““Complement component | 2.227 | ““lp01_sig””” | | |
| | | C8 beta chain”” | | | | |
| “12 | ““P20762”” | ““Ig gamma-2C chain C region”” | 2.221 | ““lp01_sig””” | | |
| “13 | ““A0A8I6AQD7”” | ““Ig-like domain- | 2.22 | ““lp01_sig””” | | |
| | | containing protein”” | | | | |
| “14 | ““A0A9K3Y6Z7”” | ““Fibrinogen alpha chain”” | 2.142 | ““lp01_sig””” | | |
| “15 | ““Q03626”” | ““Murinoglobulin-1”” | 2.114 | ““lp01_sig””” | | |
| “16 | ““P02651”” | ““Apolipoprotein A-IV”” | 2.107 | ““lp01_sig””” | | |
| “17 | ““P08649”” | ““Complement C4”” | 2.087 | ““lp01_sig””” | | |
| “18 | ““A6J8R3”” | ““Apolipoprotein C-I | isoform CRA_a”” | 1.944 | ““lp01_sig””” | |
| “19 | ““A6ID81”” | ““Peroxiredoxin-6”” | 1.713 | ““lp01_sig””” | | |
| “20 | ““A0A8I5ZTK7”” | ““Ig-like domain- | 1.568 | ““lp01_sig””” | | |
| | | containing protein”” | | | | |
| “21 | ““M0R7Q2”” | ““Ig-like domain- | 1.525 | ““lp01_sig””” | | |
| | | containing protein”” | | | | |
| “22 | ““A0A8I6AMC9”” | ““C3 and PZP-like | alpha-2- | 1.518 | ““lp01_sig””” | |
| | | | macroglobulin | | | |
| | | | domain | | | |
| | | | containing 8”” | | | |
| “23 | ““A6ISB2”” | ““Proteasome subunit beta”” | 1.504 | ““lp01_sig””” | | |
| “24 | ““F1LYQ4”” | ““Ig-like domain- | 1.498 | ““lp01_sig””” | | |
| | | containing protein”” | | | | |
| “25 | ““P31211”” | ““Corticosteroid- | 1.395 | ““lp01_sig””” | | |
| | | binding globulin”” | | | | |
| “26 | ““A6KF54”” | ““RCG64255 (Fragment)”” | 1.368 | ““lp01_sig””” | | |
| “27 | ““A0A0G2JV24”” | ““Thrombospondin 1”” | 1.367 | ““lp01_sig””” | | |
| “28 | ““Q5M7T5”” | ““Antithrombin-III”” | 1.267 | ““lp01_sig””” | | |
| “29 | ““A0A8I6GKX5”” | ““Inter-alpha-trypsin | 1.232 | ““lp01_sig””” | | |
| | | inhibitor heavy chain H3”” | | | | |
| “30 | ““Q4QQW0”” | ““Igh-6 protein (Fragment)”” | 1.208 | | | |
| “31 | ““A0A8L2Q996”” | ““Elongation factor 1-alpha”” | 11.43 | ““sang_sig””” | | |
| “32 | ““A0A8L2QLW7”” | ““Hemoglobin alpha | adult chain 1”” | 4.892 | ““sang_sig””” | |
| “33 | ““P23562”” | ““Band 3 anion | 3.524 | ““sang_sig””” | | |
| | | transport protein”” | | | | |
| “34 | ““P01836”” | ““Ig kappa chain C region | A allele”” | 3.144 | ““sang_sig””” | |
| “35 | ““F1LWD0”” | ““Ig-like domain- | 2.738 | ““sang_sig””” | | |
| | | containing protein”” | | | | |
| “36 | ““P06765”” | ““Platelet factor 4”” | 2.148 | ““sang_sig””” | | |
| “37 | ““Q7TMC0”” | ““Mannose- | 1.953 | ““sang_sig””” | | |
| | | binding protein C”” | | | | |
| “38 | ““Q9JJM7”” | ““Platelet glycoprotein | 1.798 | ““sang_sig””” | | |
| | | Ib beta chain”” | | | | |
| “39 | ““G3V7N9”” | ““Adiponectin a”” | 1.774 | ““sang_sig””” | | |
| “40 | ““A0A8I6GGN2”” | ““Integrin subunit | 1.713 | ““sang_sig””” | | |
| | | alpha 2b”” | | | | |
| “41 | ““P02770”” | ““Albumin”” | 1.708 | ““sang_sig””” | | |
| “42 | ““A0A3B0J380”” | ““Complement C1q | 1.675 | ““sang_sig””” | | |
| | | subcomponent subunit A”” | | | | |
| “43 | ““A0A0G2K4H7”” | ““Keratin 78”” | 1.665 | ““sang_sig””” | | |
| “44 | ““O7TMA9”” | ““Aa1249”” | 1.653 | ““sang_sig””” | | |
| “45 | ““A0A0G2KA54”” | ““Kininogen 1”” | 1.429 | ““sang_sig””” | | |
| “46 | ““A0A023IKI3”” | ““Proteasome subunit beta”” | 1.411 | ““sang_sig””” | | |
| “47 | ““A0A8I6AMP2”” | ““proteasome | 1.387 | ““sang_sig””” | | |
| | | endopeptidase complex”” | | | | |
| “48 | ““A0A8I6A4U0”” | ““Ig-like domain- | 1.356 | ““sang_sig””” | | |
| | | containing protein”” | | | | |
| “49 | ““A0A8I6ANU1”” | ““Ig-like domain- | 1.318 | ““sang_sig””” | | |
| | | containing protein”” | | | | |
| “50 | ““A0A8I5ZVK5”” | ““Plasminogen”” | 1.236 | ““sang_sig””” | | |
| “51 | ““A6IM16”” | ““Pregnancy-zone protein”” | 1.234 | ““sang_sig””” | | |
| “52 | ““A0A0G2KB42”” | ““Clusterin”” | 19.507 | ““shared_sig””” | | |
| “53 | ““F1LM47”” | ““Succinate--CoA | mitochondrial”” | 14.896 | ““shared_sig””” | |
| | | ligase [ADP- | | | | |
| | | forming] | | | | |
| | | subunit beta | | | | |
| “54 | ““A6KQR3”” | ““Complement C3”” | 13.995 | ““shared_sig””” | | |
| “55 | ““P20761”” | ““Ig gamma-2B | 9.041 | ““shared_sig””” | | |
| | | chain C region”” | | | | |
| “56 | ““B2RYM3”” | ““Inter-alpha-trypsin | 7.105 | ““shared_sig””” | | |
| | | inhibitor heavy chain H1”” | | | | |
| “57 | ““P23764”” | ““Glutathione peroxidase 3”” | 6.204 | ““shared_sig””” | | |
| “58 | ““A0A0G2JW41”” | ““Ig-like domain- | 5.519 | ““shared_sig””” | | |
| | | containing protein”” | | | | |
| “59 | ““A6KUH4”” | ““RCG47027 (Fragment)”” | 4.714 | ““shared_sig””” | | |
| “60 | ““F1LZ11”” | ““Ig-like domain- | 4.521 | ““shared_sig””” | | |
| | | containing protein”” | | | | |
| “61 | ““Q4VBH1”” | ““Ighg protein”” | 3.459 | ““shared_sig””” | | |
| “62 | ““A6KDI1”” | ““RCG21092 (Fragment)”” | 3.343 | ““shared_sig””” | | |
| “63 | ““A0A0G2K4H9”” | ““Keratin 12”” | 3.123 | ““shared_sig””” | | |
| “64 | ““Q7TP24”” | ““Signal recognition | 2.997 | ““shared_sig””” | | |
| | | particle receptor subunit | | | | |
| | | beta”” | | | | |
| “65 | ““A6JS43”” | ““Histidine- | 2.996 | ““shared_sig””” | | |
| | | rich glycoprotein”” | | | | |
| “66 | ““Q5M7V3”” | ““LOC367586 protein”” | 2.808 | ““shared_sig””” | | |
| “67 | ““A0A8I5ZPF0”” | ““Haptoglobin”” | 2.646 | ““shared_sig””” | | |
| “68 | ““A0A8I6ALZ6”” | ““Ig-like domain- | 2.435 | ““shared_sig””” | | |
| | | containing protein”” | | | | |
| “69 | ““A0A0G2K151”” | ““Apolipoprotein E”” | 2.293 | ““shared_sig””” | | |
| “70 | ““A0A0G2JVQ5”” | ““Kininogen 2”” | 2.182 | ““shared_sig””” | | |
| “71 | ““A0A8I5ZZZ2”” | ““Serpin family A member 4”” | 2.049 | ““shared_sig””” | | |
| “72 | ““D3ZAN3”” | ““Glucosidase | 1.972 | ““shared_sig””” | | |
| | | II alpha subunit”” | | | | |
| “73 | ““A0A8I6AD89”” | ““Ig-like domain- | 1.757 | ““shared_sig””” | | |
| | | containing protein”” | | | | |
| “74 | ““P20059”” | ““Hemopexin”” | 1.611 | ““shared_sig””” | | |
| “75 | ““F1M7H2”” | ““Polymeric | 1.609 | ““shared_sig””” | | |
| | | immunoglobulin receptor”” | | | | |
| “76 | ““A0A0G2JSW3”” | ““Hemoglobin subunit beta”” | 1.527 | ““shared_sig””” | | |
| “77 | ““A6JJH0”” | ““RCG60999 | isoform CRA_a”” | 1.512 | ““shared_sig””” | |
| “78 | ““A0A8I6GER1”” | ““Kininogen 2”” | 1.485 | ““shared_sig””” | | |
| “79 | ““A0A3B0IYY4”” | ““Adiponectin b”” | 1.368 | ““shared_sig”” ” | | |

Hierarchical clustering further emphasized the stark difference between LP01 and SANG protein corona where they shared abundances and composition of one cluster containing 6 of 115 proteins (FIG. 36, panel d). Proteins, abundances, and their membership in one of eight clusters are detailed in Table 2.

TABLE 2
List of proteins, abundances, and membership in one of eight clusters.

| “” | “description” | “cluster” | | |
|---|---|---|---|---|
| “1” | “Elongation factor 1- | “cluster1” | | |
| | alpha” | | | |
| “2” | “Histidine-rich | “cluster1” | | |
| | glycoprotein” | | | |
| “3” | “Keratin 78” | “cluster2” | | |
| “4” | “Kininogen 2” | “cluster2” | | |
| “5” | “Myosin regulatory | “cluster2” | | |
| | light polypeptide 9” | | | |
| “6” | “Carboxylic ester | “cluster2” | | |
| | hydrolase” | | | |
| “7” | “Platelet factor 4” | “cluster2” | | |
| “8” | “Band 3 anion | “cluster2” | | |
| | transport protein” | | | |
| “9” | “Complement C1q | “cluster3” | | |
| | subcomponent | | | |
| | subunit A” | | | |
| “10” | “Ig-like domain- | “cluster3” | | |
| | containing protein” | | | |
| “11” | “Tropomyosin beta | “cluster3” | | |
| | chain” | | | |
| “12” | “Ig kappa chain C | A allele” | “cluster3” | |
| | region | | | |
| “13” | “Aa1249” | “cluster3” | | |
| “14” | “Hemoglobin subunit | “cluster4” | | |
| | beta” | | | |
| “15” | “UTP--glucose-1- | “cluster4” | | |
| | phosphate | | | |
| | uridylyltransferase” | | | |
| “16” | “Dihydrolipoyllysine- | mitochondrial” | “cluster4” | |
| | residue | | | |
| | succinyltransferase | | | |
| | component of 2- | | | |
| | oxoglutarate | | | |
| | dehydrogenase | | | |
| | complex | | | |
| “17” | “Hemoglobin alpha | adult chain 1” | “cluster4” | |
| “18” | “ATP synthase | “cluster4” | | |
| | subunit beta” | | | |
| “19” | “Keratin 12” | “cluster5” | | |
| “20” | “Tetraspanin” | “cluster5” | | |
| “21” | “Arachidonate 12- | isoform | “cluster5” | |
| | lipoxygenase | CRA_b” | | |
| | (Predicted) | | | |
| “22” | “Complement | alpha | isoform | “cluster5” |
| | component 4 binding | | CRA_a” | |
| | protein | | | |
| “23” | “Coagulation factor | “cluster5” | | |
| | X” | | | |
| “24” | “Polymeric | “cluster5” | | |
| | immunoglobulin | | | |
| | receptor” | | | |
| “25” | “Inter alpha-trypsin | heavy chain 4” | “cluster5” | |
| | inhibitor | | | |
| “26” | “CD5 antigen-like” | “cluster5” | | |
| “27” | “Complement | “cluster5” | | |
| | component C9” | | | |
| “28” | “Fibrinogen-like 2” | “cluster5” | | |
| “29” | “Afamin” | “cluster5” | | |
| “30” | “Ras-related protein | “cluster5” | | |
| | Rap-1b” | | | |
| “31” | “Apolipoprotein A-I” | “cluster6” | | |
| “32” | “Thrombospondin 1” | “cluster6” | | |
| “33” | “Kininogen 2” | “cluster6” | | |
| “34” | “Ig-like domain- | “cluster6” | | |
| | containing protein” | | | |
| “35” | “Serine (or cysteine) | clade A | member 3N” | “cluster6” |
| | peptidase inhibitor | | | |
| “36” | “Apolipoprotein E” | “cluster6” | | |
| “37” | “Haptoglobin” | “cluster6” | | |
| “38” | “Ig-like domain- | “cluster6” | | |
| | containing protein” | | | |
| “39” | “Ig-like domain- | “cluster6” | | |
| | containing protein” | | | |
| “40” | “Plasminogen” | “cluster6” | | |
| “41” | “Serpin family A | “cluster6” | | |
| | member 4” | | | |
| “42” | “Complement C1s” | “cluster6” | | |
| “43” | “Ig-like domain- | “cluster6” | | |
| | containing protein” | | | |
| “44” | “Ig-like domain- | “cluster6” | | |
| | containing protein” | | | |
| “45” | “C3 and PZP-like | alpha-2- | “cluster6” | |
| | | macroglobulin | | |
| | | domain | | |
| | | containing 8” | | |
| “46” | “Fetuin B” | “cluster6” | | |
| “47” | “Integrin subunit | “cluster6” | | |
| | alpha 2b” | | | |
| “48” | “Inter-alpha-trypsin | “cluster6” | | |
| | inhibitor heavy chain | | | |
| | H3” | | | |
| “49” | “Myosin-9” | “cluster6” | | |
| “50” | “Proteasome subunit | “cluster6” | | |
| | beta” | | | |
| “51” | “Fibrinogen alpha | “cluster6” | | |
| | chain” | | | |
| “52” | “Alpha-1-B | isoform | “cluster6” | |
| | glycoprotein | CRA_a” | | |
| “53” | “Proteasome subunit | “cluster6” | | |
| | beta” | | | |
| “54” | “Fibrinogen | B beta | isoform | “cluster6” |
| | | polypeptide | CRA_b” | |
| “55” | “Protein AMBP” | “cluster6” | | |
| “56” | “Apolipoprotein C-I | isoform | “cluster6” | |
| | | CRA_a” | | |
| “57” | “RCG60999 | isoform | “cluster6” | |
| | | CRA_a” | | |
| “58” | “Tubulin alpha chain” | “cluster6” | | |
| “59” | “Complement C3” | “cluster6” | | |
| “60” | “Inter-alpha-trypsin | “cluster6” | | |
| | inhibitor heavy chain | | | |
| | H1” | | | |
| “61” | “Glucosidase II alpha | “cluster6” | | |
| | subunit” | | | |
| “62” | “Ig-like domain- | “cluster6” | | |
| | containing protein” | | | |
| “63” | “Ig-like domain- | “cluster6” | | |
| | containing protein” | | | |
| “64” | “Joining chain of | “cluster6” | | |
| | multimeric IgA and | | | |
| | IgM” | | | |
| “65” | “Adiponectin a” | “cluster6” | | |
| “66” | “Ig-like domain- | “cluster6” | | |
| | containing protein” | | | |
| “67” | “Tubulin beta chain” | “cluster6” | | |
| “68” | “Hemoglobin subunit | “cluster6” | | |
| | beta-1” | | | |
| “69” | “Apolipoprotein A- | “cluster6” | | |
| | IV” | | | |
| “70” | “Complement C4” | “cluster6” | | |
| “71” | “Alpha-1-inhibitor 3” | “cluster6” | | |
| “72” | “Hemopexin” | “cluster6” | | |
| “73” | “Ig gamma-2C chain | “cluster6” | | |
| | C region” | | | |
| “74” | “Glutathione | “cluster6” | | |
| | peroxidase 3” | | | |
| “75” | “Corticosteroid- | “cluster6” | | |
| | binding globulin” | | | |
| “76” | “Complement | “cluster6” | | |
| | component C8 beta | | | |
| | chain” | | | |
| “77” | “Igh-6 protein | “cluster6” | | |
| | (Fragment)” | | | |
| “78” | “Ighg protein” | “cluster6” | | |
| “79” | “Antithrombin-III” | “cluster6” | | |
| “80” | “LOC367586 | “cluster6” | | |
| | protein” | | | |
| “81” | “Complement C4B | “cluster6” | | |
| | (Chido blood group)” | | | |
| “82” | “Proteasome subunit | “cluster7” | | |
| | beta” | | | |
| “83” | “Ig-like domain- | “cluster7” | | |
| | containing protein” | | | |
| “84” | “Coagulation factor | “cluster7” | | |
| | V” | | | |
| “85” | “Clusterin” | “cluster7” | | |
| “86” | “Tubulin beta chain” | “cluster7” | | |
| “87” | “Ig-like domain- | “cluster7” | | |
| | containing protein” | | | |
| “88” | “Ig-like domain- | “cluster7” | | |
| | containing protein” | | | |
| “89” | “Ig-like domain- | “cluster7” | | |
| | containing protein” | | | |
| “90” | “Ig-like domain- | “cluster7” | | |
| | containing protein” | | | |
| “91” | “Peroxiredoxin-6” | “cluster7” | | |
| “92” | “RCG21092 | “cluster7” | | |
| | (Fragment)” | | | |
| “93” | “Fibronectin” | “cluster7” | | |
| “94” | “RCG47027 | “cluster7” | | |
| | (Fragment)” | | | |
| “95” | “Succinate--CoA | mitochondrial” | “cluster7” | |
| | ligase [ADP- | | | |
| | forming] subunit beta | | | |
| “96” | “Ig-like domain- | “cluster7” | | |
| | containing protein” | | | |
| “97” | “Ig-like domain- | “cluster7” | | |
| | containing protein” | | | |
| “98” | “Ig-like domain- | “cluster7” | | |
| | containing protein” | | | |
| “99” | “Fibrinogen gamma | “cluster7” | | |
| | chain” | | | |
| “100” | “Ig gamma-2B chain | “cluster7” | | |
| | C region” | | | |
| “101” | “Murinoglobulin-1” | “cluster7” | | |
| “102” | “Alpha-actinin-1” | “cluster7” | | |
| “103” | “Albumin” | “cluster8” | | |
| “104” | “Actin | cytoplasmic 1” | “cluster8” | |
| “105” | “Adiponectin b” | “cluster8” | | |
| “106” | “Integrin beta” | “cluster8” | | |
| “107” | “Filamin A” | “cluster8” | | |
| “108” | “Integrin subunit | “cluster8” | | |
| | alpha 6” | | | |
| “109” | “Myosin light | “cluster8” | | |
| | polypeptide 6” | | | |
| “110” | “Mannan-binding | “cluster8” | | |
| | protein” | | | |
| “111” | “Albumin” | “cluster8” | | |
| “112” | “Glycoprotein 9” | “cluster8” | | |
| “113” | “Mannose-binding | “cluster8” | | |
| | protein C” | | | |
| “114” | “Signal recognition | “cluster8” | | |
| | particle receptor | | | |
| | subunit beta” | | | |
| “115” | “Platelet glycoprotein | “cluster8” | | |
| | Ib beta chain” | | | |

Collectively, it is shown that SANGs develop a significantly less dense (in number and abundance) and compositionally unique protein corona relative to LP-01. Determining the biologic function and influence of SANGs' protein corona plays on tumor targeting will be the focus of future studies.

### Discussion

Formulated to contain positively charged groups to facilitate the carrying of oligonucleotides, core-shell polyacrylamide-based SANGs were shown to efficiently package and stabilize RNAi molecules and to deliver siRNA and miRNA to three cancer models, equally well in both mouse and rat across primary and metastatic loci. The RNAi cargo successfully sensitized drug-resistant tumors, allowing subsequent delivery of chemotherapeutic agents to have a dramatically enhanced effect in arresting tumor growth in vivo. The nanogel platform was found to be minimally toxic and well tolerated in mice, rats, and swine in a variety of simulated clinical applications. The most remarkable properties exhibited by SANGs are: 1) preferential targeting of primary and metastatic tumors (38) relative to healthy tissue, 2) prolonged tumor retention, 3) extravasation into the tumor interstitium, gaining access to cancerous cells, and 4) delivery of sufficient RNAi payloads to silence oncogene mRNA and protein expression and result in efficient tumor growth suppression. No systemically administered delivery platform of any form—polymeric, liposomal, proteinacious, or viral—with this combination of advantages is known.

It is believed that tumor targeting and retention derive from concentration-dependent “self-agglomeration” behavior demonstrated in vitro and in vivo. FIG. 12, panel g summarizes the following provisional mechanistic hypothesis. When administered intravenously, SANGs distribute to all organs proportional to the perfusion rate. However, the elevated resistance, sluggish blood flow, and increase tortuosity overrepresented in the maladaptive vasculature of tumors(39, 40) allows time for SANG particles to interact, coalesce(41, 42) and become entrained in tumors rather than other tissues or organ systems. This responsive behavior results in the establishment of high concentrations of SANGs in tumor vasculature, favoring extravasation down a concentration gradient. Once in the interstitial space (FIG. 11, panels a-c and f), cellular uptake mechanisms such as clathrin-mediated endocytosis and macropinocytosis mediate cellular internalization. In essence, the emergent aggregating behavior of SANGS—the existence of which is supported by the self-consistent results of five complimentary methods of analysis—amplifies the enhanced permeability and retention (EPR) effect to achieve tumor delivery, penetration, and retention to the entire tumor parenchyma regardless of carcinoma type(6, 43, 44). However, the potential involvement of receptor mediate processes cannot be ruled out such as multivalent glycoprotein recognition that might enable cells (e.g. CD31⁻/CD45⁻ populations) to recognize surface features of unfunctionalized SANGs. It should be noted that this would require conservation among at least four cancers and two species, and so is unlikely to play a major role.

Overall, environmentally-responsive nanostructures of the SANG type may represent an important therapeutic paradigm shift for systemic delivery of RNAi to solid tumors, as it exhibits exceptional in vivo performance and substantial therapeutic index that qualify it for advanced development in preparation for clinical applications. The delivery of other molecular payloads may also be envisioned, if the advantageous functional dynamics of SANGs is retained in such cases.

### REFERENCES

1. A. Fire et al., Potent and specific genetic interference by double-stranded RNA in Caenorhabditis elegans. nature 391, 806-811 (1998).
2. G. J. Hannon, RNA interference. nature 418, 244-251 (2002).
3. K. Tiemann, J. J. Rossi, RNAi-based therapeutics-current status, challenges and prospects. EMBO molecular medicine 1, 142-151 (2009).
4. K. D. Warner, C. E. Hajdin, K. M. Weeks, Principles for targeting RNA with drug-like small molecules. Nature reviews Drug discovery 17, 547-558 (2018).
5. B. Hu et al., Therapeutic siRNA: state of the art. Signal transduction and targeted therapy 5, 101 (2020).
6. K. A. Whitehead, R. Langer, D. G. Anderson, Knocking down barriers: advances in siRNA delivery. Nature reviews Drug discovery 8, 129-138 (2009).
7. B. Urban-Klein, S. Werth, S. Abuharbeid, F. Czubayko, A. Aigner, RNAi-mediated gene-targeting through systemic application of polyethylenimine (PEI)-complexed siRNA in vivo. Gene therapy 12, 461-466 (2005).
8. A. Wittrup et al., Visualizing lipid-formulated siRNA release from endosomes and target gene knockdown. Nature biotechnology 33, 870-876 (2015).
9. G. Sahay et al., Efficiency of siRNA delivery by lipid nanoparticles is limited by endocytic recycling. Nature biotechnology 31, 653-658 (2013).
10. S. Rietwyk, D. Peer, Next-generation lipids in RNA interference therapeutics. ACS nano 11, 7572-7586 (2017).
11. S. C. Semple et al., Rational design of cationic lipids for siRNA delivery. Nature biotechnology 28, 172-176 (2010).
12. M. P. Lokugamage et al., Mild innate immune activation overrides efficient nanoparticle-mediated RNA delivery. Advanced Materials 32, 1904905 (2020).
13. K. Paunovska et al., Analyzing 2000 in vivo drug delivery data points reveals cholesterol structure impacts nanoparticle delivery. ACS nano 12, 8341-8349 (2018).
14. K. Paunovska, D. Loughrey, J. E. Dahlman, Drug delivery systems for RNA therapeutics. Nature Reviews Genetics 23, 265-280 (2022).
15. K. Paunovska et al., A direct comparison of in vitro and in vivo nucleic acid delivery mediated by hundreds of nanoparticles reveals a weak correlation. Nano letters 18, 2148-2157 (2018).
16. J. E. Zuckerman, M. E. Davis, Clinical experiences with systemically administered siRNA-based therapeutics in cancer. Nature reviews Drug discovery 14, 843-856 (2015).
17. D. Hattab, A. M. Gazzali, A. Bakhtiar, Clinical advances of siRNA-based nanotherapeutics for cancer treatment. Pharmaceutics 13, 1009 (2021).
18. H. H. Gustafson, D. Holt-Casper, D. W. Grainger, H. Ghandehari, Nanoparticle uptake: the phagocyte problem. Nano today 10, 487-510 (2015).
19. T. Yeung et al., Membrane phosphatidylserine regulates surface charge and protein localization. Science 319, 210-213 (2008).
20. Y. Fang et al., Cleavable PEGylation: a strategy for overcoming the “PEG dilemma” in efficient drug delivery. Drug Delivery 24, 22-32 (2017).
21. M. Miteva et al., Tuning PEGylation of mixed micelles to overcome intracellular and systemic siRNA delivery barriers. Biomaterials 38, 97-107 (2015).
22. W. H. Blackburn, E. B. Dickerson, M. H. Smith, J. F. McDonald, L. A. Lyon, Peptide-functionalized nanogels for targeted siRNA delivery. Bioconjugate chemistry 20, 960-968 (2009).
23. D. Gan, L. A. Lyon, Tunable swelling kinetics in core-shell hydrogel nanoparticles. Journal of the American Chemical Society 123, 7511-7517 (2001).
24. C. D. Jones, L. A. Lyon, Synthesis and characterization of multiresponsive core-shell microgels. Macromolecules 33, 8301-8306 (2000).
25. M. Satpathy, R. Mezencev, L. Wang, J. F. McDonald, Targeted in vivo delivery of EGFR siRNA inhibits ovarian cancer growth and enhances drug sensitivity. Scientific reports 6, 36518 (2016).
26. S.-L. Liu et al., Visualizing the endocytic and exocytic processes of wheat germ agglutinin by quantum dot-based single-particle tracking. Biomaterials 32, 7616-7624 (2011).
27. T. J. Raub, M. J. Koroly, R. M. Roberts, Endocytosis of wheat germ agglutinin binding sites from the cell surface into a tubular endosomal network. Journal of cellular physiology 143, 1-12 (1990).
28. Y. Chen et al., Targeting Xkr8 via nanoparticle-mediated in situ co-delivery of siRNA and chemotherapy drugs for cancer immunochemotherapy. Nature Nanotechnology 18, 193-204 (2023).
29. M. A. Subhan, S. S. K. Yalamarty, N. Filipczak, F. Parveen, V. P. Torchilin, Recent advances in tumor targeting via EPR effect for cancer treatment. Journal of personalized medicine 11, 571 (2021).
30. J. D. Finn et al., A Single Administration of CRISPR/Cas9 Lipid Nanoparticles Achieves Robust and Persistent In Vivo Genome Editing. Cell Rep. 22, 2227-2235 (2018).
31. M. Benezra et al., Multimodal silica nanoparticles are effective cancer-targeted probes in a model of human melanoma. The Journal of clinical investigation 121, 2768-2780 (2011).
32. Q. Sheng, J. Liu, The therapeutic potential of targeting the EGFR family in epithelial ovarian cancer. British journal of cancer 104, 1241-1245 (2011).
33. E. Teplinsky, F. Muggia, EGFR and HER2: is there a role in ovarian cancer? Translat Cancer Res 4, (2015).
34. S. N. Housley et al., Cancer Exacerbates Chemotherapy-Induced Sensory Neuropathy. Cancer Research, (2020).
35. D. Bornstein, R. Pazur, The sulfur reversion process in natural rubber in terms of crosslink density and crosslink density distribution. Polymer Testing 88, 106524 (2020).
36. T. Saito, M. Yamano, K. Nakayama, S. Kawahara, Quantitative analysis of crosslinking junctions of vulcanized natural rubber through rubber-state NMR spectroscopy. Polymer Testing 96, 107130 (2021).
37. R. Hincapie et al., Multivalent Targeting of the Asialoglycoprotein Receptor by Virus-Like Particles. Small 19, 2304263 (2023).
38. D. Rosenblum, N. Joshi, W. Tao, J. M. Karp, D. Peer, Progress and challenges towards targeted delivery of cancer therapeutics. Nature communications 9, 1410 (2018).
39. J. R. Less, M. C. Posner, T. C. Skalak, N. Wolmark, R. K. Jain, Geometric resistance and microvascular network architecture of human colorectal carcinoma. Microcirculation 4, 25-33 (1997).
40. E. M. Sevick, R. K. Jain, Geometric resistance to blood flow in solid tumors perfused ex vivo: effects of tumor size and perfusion pressure. Cancer research 49, 3506-3512 (1989).
41. V. P. Chauhan, T. Stylianopoulos, Y. Boucher, R. K. Jain, Delivery of molecular and nanoscale medicine to tumors: transport barriers and strategies. Annual review of chemical and biomolecular engineering 2, 281-298 (2011).
42. D. Fukumura, R. K. Jain, Tumor microenvironment abnormalities: causes, consequences, and strategies to normalize. Journal of cellular biochemistry 101, 937-949 (2007).
43. Y. Ren et al., Targeted tumor-penetrating siRNA nanocomplexes for credentialing the ovarian cancer oncogene ID4. Science translational medicine 4, 147ra112-147ra112 (2012).
44. E. Ruoslahti, S. N. Bhatia, M. J. Sailor, Targeting of drugs and nanoparticles to tumors. Journal of cell biology 188, 759-768 (2010).

### Materials and Methods Animal Studies

Six- to eight-week-old female NOD.CB17-Prkdc^{scid} IL2rg^{tm1}/BcgenHsd (B-NDG: Envigo), NOD.CB17-Prkdc^{scid}/NCrCrl (NOD SCID), C57BL/6, and CD-1 Mice (Charles River) were maintained at the Georgia Institute of Technology's Physiological Research Laboratory in individually ventilated and watered cages kept at negative pressure. Immunodeficient animals were housed under pathogen-free conditions according to Association for Assessment and Accreditation of Laboratory Animal Care guidelines. The mice were kept in rooms on a 12 h light/dark cycle with ambient temperature between 22.8 and 23.9 °C with 30-40% relative humidity. The experiments were only performed during the light phase. Sterile food (Alfalfa Free Imaging Lab Diet) and sterile water was provided to mice ad libitum. The mice were acclimatized for at least 6 days before the beginning of experiments. The animals were randomly distributed among the experimental groups. Researchers were blinded to the animal group allocation during data acquisition. The animals were euthanized by isoflurane overdose (5%) and exsanguination.

Six-month male and eight-month old F344/NTac-Apc^{am137} (Pirc) and F344 rats (Rat Resource & Research Center), ten- to fourteen-week-old Sprague Dawley-Rag2^{em2hera}Il2rg^{em1hera}/HblCrl (SRG), Sprague Dawley (Charles River), Sprague Dawley HsdSage:SD-Rag2^{emIsage} (Rag2) (Envigo), and were maintained at the Georgia Institute of Technology in individually ventilated and watered cages kept at negative pressure. Immunodeficient animals were housed under pathogen-free conditions according to Association for Assessment and Accreditation of Laboratory Animal Care guidelines. The rats were kept in rooms on a 12 h light/dark cycle with ambient temperature between 22.8 and 23.9 °C with 30-40% relative humidity. The experiments were only performed during the light phase. Sterile food (Alfalfa Free Imaging Lab Diet) and sterile water was provided to rats ad libitum. The rats were acclimatized for at least 6 days before the beginning of experiments. The animals were randomly distributed among the experimental groups. Researchers were blinded to the animal group allocation during data acquisition. The animals were euthanized by isofluorane overdose (5%) and exsanguination.

Ten- to fourteen-week-old Sprague Dawley rats with chronically catheterized left jugular veins (Charles River) were used for pharmacokinetic and toxicity studies and handled as described above with the following exceptions. Rats were singly housed to avoid disruption of the Vascular Access Buttons™ (Instech) and catheter patency was maintained by flushing (heparinized saline 500 units/mL) and locking (heparinized dextrose: 500 units/mL in dextrose 50%) catheters every 5-7 days.

All animals were cared for according to the Georgia Institute of Technology Physiological Research Laboratory policies and under ethical guidance from the university's Institutional Animal Care and Use Committee (IACUC) following National Institutes of Health guidelines.

A sexually mature (6 month) female Domestic Yorkshire Crossbred Swine weighing 73 kg was studied at Global Center for Medical Innovation (GCMI) T3 Labs with Institutional IACUC approval. The Test Facility is accredited by the Association for the Assessment and Accreditation of Laboratory Animal Care, International (AAALAC) and registered with the United States Department of Agriculture to conduct research in laboratory animals. Before treatment, a physical exam and clinical assessment was performed. After swine was deeply anesthetized an acutely placed jugular catheter was placed with ultrasound guidance for slow infusion of SANGs and serial blood sampling. At the conclusion of the study, the swine was euthanized via intravenous administration of KCL 1-2 meq/kg under a deep plane of anesthesia.

### Tumor Cell Lines and Cancer Model Induction

HEY-A8 gifted by Dr. Gordon B. Mills, (MD Anderson Cancer Center, Houston, TX) were stably transfected with pGL4.51 Luciferase Reporter Vector (Promega) as has been previously reported. The resulting HEY-A8-F8 cell lines and MDA-MB-231-luc2 (SC059-Puro) TNBC cell lines (GeneTarget) were maintained in RPMI 1640 (Mediatech) supplemented with 10% FBS (Fetal Bovine Serum; Atlanta Biologicals) and 1% antibiotic-antimycotic solution (Mediatech). Media were supplemented with Geneticin G418 sulphate (GIBCO) and Puromycin (GIBCO) selective agents to maintain Luciferase expression. Adherent monolayer cultures were maintained at 37 °C in 5% CO2.

For establishment of mouse TBNC model, 1×10⁴ cells/50 μL saline were subcutaneously injected into the fourth mammary fat pads using a 27 gauge×½ inch needle. For establishment of mouse primary OC model, 4×10⁵ cells/100 μL saline were injected into the right ovary. For establishment of mouse metastatic OC model, 4×10⁵ cells/100 μL saline were injected into the right lower quadrant of the abdominal cavity using a 27 gauge×½ inch needle. For establishment of rat TBNC and OC models between 1-2×10⁶ cells/100 μL were injected in the right lower quadrant of the abdominal cavity using a 27 gauge×½ inch needle.

### SANG Synthesis

SANGs were synthesized via emulsion precipitation polymerization as previously described(W. H. Blackburn, L. A. Lyon, Size-controlled synthesis of monodisperse core/shell nanogels. Colloid and Polymer Science 286, 563-569 (2008)). In a typical core nanogel synthesis, N-iso-propylmethacrylamide (NIPMAm) monomer (117.8 mM), N, N′-methylene-bis (acrylamide) (BIS) cross linker (6.2 mM) and sodium dodecyl sulfate (SDS) surfactant (8 mM) were dissolved in deionized H₂O. The solution was stirred under nitrogen, brought to 70 °C, and treated with 0.5 mL of a 10 mg/mL aqueous ammonium persulfate (APS) solution to initiate polymerization (800 mM). The reaction was carried out at 70 °C for 4 hours with vigorous stirring under nitrogen. Crude core particle reaction solution was 0.2 μm filtered. Core particle synthesis was verified by DLS as having unimodal distribution between 47-57 nm. The core nanogels were used as seed for the addition of a shell layer. A mixture of NIPMAm, BIS, and aminopropyl methacrylate (APMA, Polysciences) prepared in dH₂O (0.25 mM) was added to core seed. When the temperature stabilized at 70 °C, the reaction was initiated by adding a 0.5 mL aliquot of 0.05 M APS, and was allowed to proceed for 4 hours under N₂ gas. After cooling to room temperature, the reaction mixture was filtered through a 0.2 μm filter and the size of the crude core-shell nanogels measured by multi-angle dynamic light scattering. SANGs were then purified and concentrated using a VivaFlow 50R 100 kDa MWCO tangential-flow filtration (TFF) cartridge (Sartorius, circulation flow rate 10 mL/min, transmembrane pressure~2 bar), retaining the SANGS within the retentate while allowing small-molecule reagents to be removed. During the initial cleaning phase, the circulating volume was maintained by continual addition of dH₂O, corresponding to 10× volume of washing (for example, a 50 mL reaction volume was washed with 500 mL of dH₂O). After cleaning, no additional dH₂O was added and the solution was allowed to concentrate by ~10-fold. Purified SANGs were re-checked with DLS and then concentrated SANG solutions were then flash frozen and lyophilized under vacuum. When reconstituted in aqueous media, SANGS swell back to their original size.

### SANGs Labeling

SANGs that were used for in vitro delivery and in vivo biodistribution studies were labeled with AF-488-NHS, Texas Red-NHS, or LiCOR IRDye® 800RS NHS ester following the manufacturer's protocol for labeling high molecular weight molecules, depending on the study needs. For example, SANGs were reconstituted in pH 8.4 PBS at 52 nM and were incubated with 1 mM LiCOR IRDye® 800RS NHS ester overnight at room temperature. Unreacted dyes were then removed by Pierce® Zeba™ Desalting Spin Columns following the manufacture's protocol.

siRNA Labeling

siRNA species were fluorescently labeled per manufacturer's instructions using the Ulysis Alexa Fluor 647 Nucleic Acid Labeling Kit. Pre-conjugated Cy3-siRNA was purchased from Invitrogen.

### Dynamic Light Scattering and Multiangle Laser Light Scattering Measurements

SANGs were prepared as described for in vivo usage with a final concentration of 1.0 mg·mL⁻¹ in PBS. For size analysis, a 10 μL aliquot was diluted into 990 μL dH₂O or saline at pH 7.4 and analyzed using a Malvern Zetasizer Nano ZS instrument and validated with DynaPro® NanoStar® instrument. For zeta potential measurements, a 0.4 mL aliugot of the standard particle solution was diluted in 4.6 mL dH₂O at pH 7.4 and loaded into a Malvern capillary for analysis. Concentration-dependent changes in hydrodynamic size were measured by increasing the initial dry weight of SANGs used to generate starting solutions. Dilution challenged studies by dynamic light scattering (DynaPro® NanoStar®) were accomplished by diluting solutions from a starting concentration of 0.1 mg/mL in PBS.

Multiangle laser light scattering (MALLS) (Wyatt Technology Corporation, Santa Barbara, CA) was used to determine the molecular weight for all SANGs at 25 °C. Major physiochemical measurements were independently validated by the National Cancer Institute's National Characterization Laboratory (NCL).

### Electron Microscopy Measurements

For conventional TEM, three (3) microliters of the samples were prepared for negative stain using formvar/carbon 200 mesh copper grids. The grids were previously glow discharged (negative charge) for 15 s using a Glo-Qube Plus glow discharge system (Quorum Tech). Samples were then stained with 2% uranyl acetate for one minute. The images were collected using a JEOL JEM-1400 transmission electron microscope operating at 80 keV equipped with a Gatan US1000 2k×2k CCD camera.

For cryo-TEM, three (3) microliters of the samples were placed on 300 mesh copper grids (carbon substrate with 1.2 m holes spaced by 1.3 m, Quantifoil Micro Tools). The grids were previously glow discharged (negative charge) for 15 s using a Glo-Qube Plus glow discharge system (Quorum Tech). The samples were blotted with filter paper for either 3.0 s at room temperature and 100% humidity and vitrified by rapid immersion in liquid ethane using a Vitrobot Mark IV (Thermo Fisher Scientific). The cryogenic electron microscopy (cryo-EM) grids were stored in liquid nitrogen prior to data collection.

Cryo-grids were transferred to a Gatan 626 cryo holder maintained at −178 °C. Cryo-EM images were acquired using a JEOL JEM1400 transmission electron microscope operating at 80 keV. The micrographs were collected at a nominal magnification of ×20,000, on a 2,048×2,048 pixels charge-coupled device camera (UltraScan 1000, Gatan), yielding a pixel size of 5.1 Å. The overview images were collected at ×2,000 (52 Å per pixel). Dilution challenged studies were accomplished by ten-fold dilution in dH₂O of samples previously analyzed by TEM.

### Liquid Phase TEM

Commercially available silicon nitride microchips (Protochips Inc.) were used for imaging of soft nanohydrogels with TEM. At all concentrations, liquid cell top chips (EPT-52W-10) were used. At low concentration, 0.0263 μM, bottom chips (EPB-52BNS-10) with a static spacer size of 50 nm were used while at medium and high concentrations, 0.105 μM and 0.210 μM respectively, bottom chips (EPB-52DNS-10) with a static spacer size of 150 nm was used to account for the increase in structure size that is dependent on concentration. All chips underwent a 2-minute immersion in acetone to remove the photoresistant coating and subsequently immersed in ethanol for another 2 minutes. Excess ethanol was blotted off with filter paper and the chips were further dried by flowing N2 gas across the surface. Following the cleaning, the chips were surface treated by glow discharge in a PELCO easiGlow system at 0.39 mBar, 15 mA for 45 seconds and held for 10 seconds to render the surfaces hydrophilic and to increase wettability. The chips were then assembled into a Poseidon Select holder with 0.6 μL of sample.

Experiments began with either 0.5 or 1 mg of lyophilized soft nanohydrogels. Samples were then diluted to the desired concentration with Millipore DI water. To avoid larger particles obstructing the hermetic seal of top and bottom microchips, the diluted samples were filtered using a 0.2 μm syringe filter and then added to the liquid cell.

In-situ experiments were conducted on a FEI Tecnai F30 TEM with a thermal field emission gun operating at 200 kV in the Materials Characterization Facility of the Institute for Matter and Systems at Georgia Institute of Technology. Videos of the soft hydrogels were collected using a Gatan Oneview Camera and recorded using the Digital Micrograph software. The electron beam dose rate was calibrated to 30 e-/Å2s at a camera magnification of 19.5 kx with an in-house Digital Micrograph script. All videos were collected at 4096-by-4096 pixels and a frame rate of 15 frames per second. Corresponding images were binned by summing with Digital Micrograph to a frame rate of 1 frame per second to increase contrast between soft nanohydrogels and the background.

### Particle Analysis

All images were analyzed using the following procedure through ImageJ: (1) each raw grayscale TEM image was converted to a binary image (Image→Adjust→Threshold, with a lower cutoff of ≈0 and an upper cutoff of between 109-178 depending on the image contrast and Dark background option); (2) each resulting binary image was segmented using the built-in ImageJ watershed routine (Process→Binary→Watershed); (3) the sizes for all segmented particles were obtained using ImageJ's particle analysis package (Analyze→Analyze Particles, with size range going from 100 square pixel units to infinity, circularity from 0 to 1, showing bare outline of particles); (4) the diameter for each particle was computed from the area output by ImageJ under a spherical approximation. This process was repeated over multiple experimental trials (N=5) for each condition.

SANGs preparation and RNAi loading for in vitro and in vivo studies

Before delivery to cell cultures or to animals, SANGs were either reconstituted in sterile saline (0.5%) for delivery studies (FIG. 8, panels g-h) and biodistribution (FIG. 11) or were loaded with RNAi payloads for efficacy and delivery studies (FIGS. 9, 10, 11, 12) as our group has previously described(2). Briefly, lyophilized SANGs were reconstituted in RNAi solutions (20 M in sterile saline 0.5%) to a final concentration of 16-17 μg siRNA·mg⁻¹ of SANGs. Under these conditions, the swelling of the SANGs particles almost completely incorporates all of the solution volume, thereby loading the payload in the particles with high efficiency. SANGs were incubated at room temperature for 60 min before being loaded into syringes for i.v. delivery.

### Entrainment of RNAi in SANGs

Encapsulation efficiency was determined via ultracentrifugation of the SANG loading solution and measurement of supernatant siRNA concentration by UV-vis spectroscopy (Shimadzu UV-1601). The moles of siRNA in the loading solution and in the supernatant were determined via interpolation from a separately constructed standard curve of absorbance vs concentration. The encapsulation efficiency (EE) of the system could then be calculated through analysis of the amount of siRNA in the loading solution and the remaining moles of siRNA in the supernatant after SANG swelling was complete, as illustrated by equation 1 and in similar encapsulation experiments(W. H. Blackburn, E. B. Dickerson, M. H. Smith, J. F. McDonald, L. A. Lyon, Peptide-functionalized nanogels for targeted siRNA delivery. Bioconjugate chemistry 20, 960-968 (2009)).

$$EE = \frac{{m}_{siRNA loading} - {m}_{siRNA supernatant}}{{m}_{siRNA loading}} \times 100 \qquad ( 1 )$$

### SANG Delivery, Internalization, and Endosomal Escape Measurements

Labeled SANGs with or without RNAi payloads (depending on experimental design) were reconstituted in culture media and transferred to well plates (Ibidi Cat.No: 80807) containing 2×10⁴ cells/well and then incubated at 37 °C and 5% CO2. After the appropriate incubation length, cells were either subjected to live cell imaging in a temperature and CO2 chambered microscope or fixed with 4% paraformaldehyde for 15 min at room temperature, washed 3×(5 mins per wash) in 0.01M PBS, then permeabilized treated with 0.3% Triton X in 0.01M PBS, and stained following immunofluorescence protocol described below.

For cell uptake mechanism studies, cells were exposed to several different concentrations of the inhibitors: NaN₃(0, 25, 50 M), Cpz hydrochloride (0, 50, 100, 200 M), Mpcd (0, 0.5, 1, 2 mM), CytD (0, 50,100, 200 M), and LatA (0, 2, 4 M). For endosomal escape studies, both fluorescently labeled wheat germ agglutinin and CellLight™ Lysosomes-RFP, BacMam 2.0 were used to track endosome and lysosomal populations. The degree of colocalization was quantified based upon Pearson's correlation coefficient (R) using ZEN blue (Zeiss) colocalization module. Here, regions of interest (ROIs) were drawn around each individual cell based on membrane staining (actin or lectin derivatives). Costes algorithm then determined the thresholds of two fluorescent channels at a time, the software automatically calculated pixel intensity spatial overlap coefficients between them using colocalization coefficients described by the number of pixels colocalized in one fluorescent channel divided by the total number of pixels meaning values approach 0 as fluorophores are less colocalized.

### Cell Isolation, Staining and Flow Cytometry of Organs.

Cells were isolated 3 hours after i.v. administration of LNP-LP01 formulated to carry AlexaFluor-647 labeled siRNA against EGFR (n=3 biologically independent experiments), SANGs formulated to carry AlexaFluor-647 labeled siRNA against EGFR (n=4 biologically independent experiments), or PBS control. Animals were immediately transcardially perfused with 5 mL of 1×PBS. Organs were isolated immediately following perfusion. Livers and Kidney were cut and then placed in a digestive enzyme solution with collagenase type I (Sigma-Aldrich), collagenase type XI (Sigma-Aldrich), and hyaluronidase (Sigma-Aldrich) at 37 °C and 500 rpm for 30 minutes. Digested tissues were passed through a 70 m filter. Cells were stained to identify specific cell populations using flow cytometry. Antibody clones used for staining were anti-CD31 (390, BioLegend), antiCD45.2 (104, BioLegend), Cell populations were first gated for non-debris (forward scatter versus side scatter), then gated for singlets (forward scatter versus forward scatter width) and viable cells (Zombie). The viable, single cells were analyzed for AlexaFluor-647 positivity to determine the percentage of AlexaFluor-647 positive cells in each sample. The AlexaFluor-647 median fluorescence intensity was normalized to the autofluorescence of each tissue type, as determined using samples from PBS injected mice. Representative gating strategies for liver, and kidney cell populations are included in FIG. 29.

### Lipid Nanoparticle (LNP-LP01) Formulation and Characterization.

LNP-LP01 were formulated in a Precision Nanosystems Ignite system by mixing siRNA against EGFR (Invitrogen 4390826) conjugated with Alexa Fluor 647 in citrate buffer with the stereopure ionizable lipid LP01, DMG-PEG2K (Avanti Lipids: #880151), cholesterol (Avanti Lipids #700100), and DSPC (Avanti Lipids #860365) in 100% ethanol. They were mixed at a total flow rate of 12 mL/min. LNP hydrodynamic diameter was measured using dynamic light scattering (DLS, DynaPro Plate Reader III, Wyatt Technology). LNPs were diluted in sterile 1×PBS to a concentration of ~0.08 μg/mL and analyzed. Their diameter was ______ and monodisperse. They were then dialyzed in 1× phosphate buffered saline (PBS, Invitrogen) and sterile filtered with a 0.22 m filter.

### Laser Scanning Confocal Microscopy

Tissue sections were imaged using laser scanning confocal microscopy (LSCM) (Zeiss Biosystems, 900). Tile scans images of large tissue or well areas were acquired with a 20× objective (N.A.=0.8). Z stacks of tissue sections or high-resolution cell imaging (well plates) were acquired with a 63× objective (N.A.=1.4) at a step size of 0.5 m. Laser strength, gain, and other critical image acquisition parameters were help constant when imaging across treatment groups. For each 8-well plate experiment, a negative and positive control group were included on which the imaging parameters are first set. Stacks of images were processed and analyzed using Imaris (Bitplane) or imageJ (NIH) imaging software. Figures present images as either flat maximal projections of the sum of the z-axis optical slices or three-dimensional rendering of z stacking of two-dimensional scanning images from different depths. Analyses were performed by an investigator blinded to treatment group and was determined by measuring the mean fluorescence intensity observed on the flat maximal projections of the z-stacks images respectively.

### Cell Toxicity Assay.

The toxicity of various unloaded and loaded SANGs were determined by Tox 8 Assay as previously described. Briefly, cells incubated for overnight with unloaded and loaded SANGs (FIG. 1) then washed with PBS, the medium was replaced, and the cells were incubated for an additional 72 h in medium. The Tox 8 reagent (Sigma) was added to the cells according to the manufacturer's instructions. The fluorescence at 590 nm was read after 1 h and the extent of cellular viability/proliferation determined.

### Tissue Harvesting

Following in vivo studies, animals received a lethal dose of isoflurane anesthesia (5%) until respiration rates dropped below 10 beats/min. Then, the animals were transcardially perfused with vascular rinse containing heparin followed by ice-cold 4% paraformaldehyde (PFA) in 0.1M phosphate buffer (PB, pH=7.4). Tissues were extracted and post-fixed overnight in 4% PFA at 4 °C. The following day all tissue was transferred and stored in 30% sucrose cryoprotectant solution until sectioning took place. For immediate ex vivo analysis following in vivo biodistribution studies, animals were exsanguinated without transcardial perfusion. Following immediate ex vivo imaging, organs were either (1) drop fixed in 10% NFB or 4% PFA for ~24 hrs prior to downstream histological analysis or (2) were washed with sterile saline and immediately flash frozen (fresh) in 2-methylbutane (isopentane) pre-chilled in liquid nitrogen then stored at −80 °C for PCR analysis.

### Histological Methods and Immunofluorescence

Tissue sections were sectioned at a thickness of 50 to 75 μm on a cryostat (Leica model #CM 3050 S) and collected directly onto charged (+) microscope slides for further histological processing. Details of immunohistochemistry procedures have been previously described(3). Briefly, sections from all treatment groups were processed simultaneously. Sections were washed 3×(10 mins per wash) in 0.01M PBS with 0.3% Triton X-100 (PBST) and then blocked for 1 hr in 10% normal donkey serum diluted in PBST. Sections were then incubated overnight at room temperature in primary antibodies diluted in blocking buffer (5% normal goat serum, 0.3% Triton100 in PBS). The primary antibodies used were as follow: mouse monoclonal anti-EGRF (SC-120, Santa Cruz Biotechnolgy, 1:100), mouse monoclonal anti-K167 (SC-23900, Santa Cruz Biotechnology, 1:100). The following day tissue sections were washed again 3× in PBST and immunoreactive sites were revealed with species specific fluorescent-conjugated secondary antibodies (Jackson ImmunoResearch Laboratories). Following a 2 hr incubation in the secondary mixture at room temperature, sections were washed 3×in 0.01M PBS (no triton), mounted, and coverslipped with Vectashield (Vector Labs, Newark, CA, H1000).

### PCR

PCR was performed as described previously(S. N. Housley et al., Cancer Exacerbates Chemotherapy-Induced Sensory Neuropathy. Cancer Research, (2020); L. N. Lili, L. V. Matyunina, L. Walker, B. B. Benigno, J. F. McDonald, Molecular profiling predicts the existence of two functionally distinct classes of ovarian cancer stroma. BioMed research international 2013, (2013)). Briefly, RNAs were isolated using the mRNeasy Micro KIT (Qiagen, Germantown, MD). After total RNA quantification by NanoDrop, RNA concentration and integrity was assessed on the Bioanalyzer RNA Pico Chip (Agilent Technologies, Santa Clara, CA), complementary DNA was prepared using the high-capacity complementary DNA reverse transcription kit (Applied Biosystems, Thermo Fisher Scientific). Quantitative PCR experiments were performed using the Fast Advanced Master Mix (Thermo Fisher Scientific). GAPDH was used as a reference control gene for analysis of differential expression.

### Whole-Body Luminescence and Fluorescent Imaging and Ex Vivo Imaging

Tumors were induced in groups of five mice as described above (Tumor cell lines and cancer model induction). Tumor growth was monitored by bioluminescent imaging following intraperitoneal injection of d-luciferin (15 mg/kg). Bioluminescence analysis was conducted using the Living Image software (PerkinElmer Inc.). Once tumors grew to the appropriate size, animals were randomly assigned to different experimental groups depending on the experimental design. Animals then received intravenously administered unloaded or loaded SANGs (typically 1 mg·kg⁻¹ unless otherwise noted). Controls include free FAM-siRNA, free-fluorescence dye (Texas Red-NHS ester or LiCOR IRDye® 800RS NHS Ester) and unlabeled SANGs at the same SANG equivalent doses. At 4, 24, 48, 72, and 288 h time points, the animals were imaged on black paper with IVIS SpectrumCT (PerkinElmer). After whole-body imaging, animals were euthanized. Tumors and various organs were excised for ex vivo imaging. Tumor-free wild-type mice (C57BL/6, and CD-1 Mice) injected with SANGs or free FAM-siRNA were used as control.

Quantification of ex vivo biodistribution was performed as follows. Background tissue auto-fluorescence was subtracted by normalizing all tissues to that measured in each animal's cardiac tissue, which was empirically determined to be the lowest absolute value across all measured organs. The fold-changes for each organ system were then computed. In this way all cardiac tissues had fold-change values of 1.0, and every other tissue had values greater than 1.0. This allows reliable and realistic subtraction of auto-fluorescence effects across species and strains.

### Metastatic Targeting Analysis

To quantify the specificity of metastatic targeting by SANGs, tumor BLI and SANG fluorescent images (acquired with IVIS SpectrumCT (PerkinElmer)) were imported into ImageJ software and subjected to colocalization analysis with JACoP plugin (v2.1.4) as previously described(F. P. Cordelieres, S. Bolte, in ImageJ User & Developer Conference. (2008), vol. 174, pp. 181). A total of 5 independent animals were utilized for analysis.

### Pharmacokinetics and Excretion Studies

To define the pharmacokinetics of SANGs, blood was collected in EDTA-containing tubes pre-treatment, 5 min, 0.5 h, 1 h, 2 h, 4 h, 24 h, and 48 h time points from chronically implanted left-jugular catheters. 100 μl of blood was plated from each time point for each animal on a 96well plate and imaged by IVIS SpectrumCT (PerkinElmer). Fluorescence signal was normalized within each animal based on its pre-treatment value and its maximal initial dose value then averaged across all animals (n=5)

For the excretion studies, animals were singly housed in metabolic cages and provided with food and water ad libitum. Loaded SANGs and unloaded FAM-siRNA formulations were administration i.v. via a chronically implanted left-jugular catheter at 1 mg·kg⁻. Urine and feces were collected from over 0-1, 1-3, 3-9, and 9-24 hours time periods post-administration. Fluorescent signals from urine and feces were imaged by IVIS SpectrumCT (PerkinElmer) and analyzed by Living Image Software (PerkinElmer).

### Toxicity & Tolerability Studies

Two 7 mg·kg⁻¹ doses i.v. of either (1) empty NG or (2) NG loaded with scrambled miRNA and siRNA were delivered to female CD-1 mice (5 animals/group). Daily weight changes and terminal serum blood chemistry were analyzed 14 days after the final dose in CD-1 mice.

Two clinical applications of SANGs were simulated. First, 7 mg·kg⁻¹ SANGs loaded with siRNA against EGFR were intravenously administered to female NOD-SCID mice with fully developed OC tumors. Twenty-four (24) hours after SANG dose, 10 mg·kg⁻¹ cisplatin was intraperitoneally administered. Potential test article-related effects were evaluated by the assessment of clinical signs and body weight. In separate cohorts of identically treated experimental animals, serum blood chemistry and inflammatory markers were evaluated 24 hours after cisplatin treatment.

Pirc rats (n=5 male) at least 6-month-old at initiation of dosing were treated with SANGs loaded with siRNA against EGFR followed by a 5 mg·kg⁻¹ oxaliplatin dose i.p. 24 h later. This was repeated twice weekly for four weeks. Potential repeat dose test article-related effects were evaluated by the assessment of clinical signs and body weight.

MTD study in female and male Sprague-Dawley rats were obtained from Charles River Laboratories and were 8-10 weeks old at initiation of dosing. SANGs loaded with siRNA against EGFR, was administered at 0, 7, 12, 17 mg·kg⁻¹ (3 animals/sex/group) via single intravenous injection in catharized jugular vein. Approximately, 0.3 mL of blood was obtained 6- and 24-hours post-dose from jugular catheters of conscious animals using manual restraint and was processed to serum. All groups were terminated 24 hours post dose for postmortem evaluation. Potential test article-related effects were evaluated by the assessment of clinical signs, body weight, serum chemistry (ANTECH-GLP), macroscopic observations at necropsy, and histopathology Emory University School of Medicine Department of Pathology and Laboratory Medicine).

### NMR Measurements

NMR spectra were obtained on a Bruker DRX-500 or Bruker AV3 HD-700 instrument in water-d2. All the 1H NMR experiments are reported in 6 units, parts per million (ppm), and were measured relative to the signals for residual water-d₂ (4.65 ppm). Diffusion coefficients were obtained through DOSY NMR experiments using the Bruker pulse sequence dstegp3s (2D 1H DOSY double stimulated-echo) to compensate for possible effects of convective motions. The longitudinal eddy current delay and the gradient recovery delay were kept at fixed values of 20 and 100 ms, respectively. The data from the NMR experiments were processed with the software TopSpin (Bruker), and diffusion coefficients were then obtained by integrating several 1H-signals and fitting the corresponding peaks area to fulfill the Stejskal-Tanner expression.

### In Vivo Agglomeration Measurements

SANGs were conjugated to either AF-488-NHS or Texas-Red-NHS resulting in two fluorescently distinct populations. These two populations were then i.v. administered via the lateral tail vein to Pirc rats (n=5). 48 h after administration, animals were euthanized, major organs and colorectal tumors extracted as described (Tissue Harvesting) and processed for LSCM. Tissues were labeled only with DAPI and Lectin-DyLight™ 649 (Vector Laboratory: DL-1178-1) to label nuclei and cell bodies respectively. Tissues were then imaged as described in Laser scanning confocal microscopy. The degree of colocalization was quantified based upon Pearson's correlation coefficient (R) using ZEN blue (Zeiss) colocalization module as described. 20× organ wide images and expanded 63× images were analyzed to quantify: (1) specific regions of interest (pixel-based), (2) to perform object-based colocalization analysis, and (3) quantify the size of SANG agglomerates that colocalize compared to isolated SANGs (FIG. 13, panels a-h).

### Expression and Purification of Qp VLPs

Wild-type QP particles particles were expressed and isolated as previously described (Das, S. et al. Stabilization of Near-Infrared Fluorescent Proteins by Packaging in Virus-like Particles. Biomacromolecules 21, 2432-2439 (2020)). Briefly, Chemically Competent BL21(DE3) cells were transformed with pET28CP, plated onto SOB-agar containing appropriate antibiotics, and grown overnight. After 24 h, a single well-isolated colony was inoculated into 25 mL of SOB media containing antibiotics. Following overnight growth, cultures were diluted into 500 mL of selective SOB media and incubated at 37 °C. Cultures were grown to OD600 between 0.7 and 0.9, and protein expression was induced by the addition of isopropyl β-d-1-thiogalactopyranoside (IPTG) to a final concentration of 1 mM. Following incubation for 5 h at 37 °C, cells were harvested by centrifugation (6000 rpm, 10 min) and frozen at −80 °C until processing for particle isolation.

Frozen cell pellets were resuspended in 0.1 M potassium phosphate buffer, pH 7.4, and lysed by probe sonication (10 min on total, 75 W, 5 s on, 5 s off) in an ice bath. Lysates were clarified by centrifugation (14 000 rpm, 10 min), and VLPs were precipitated from the cell lysate supernatant by addition of 27% (NH4)2SO4 (w/v) and subsequent gentle shaking at 4 °C for a minimum of 1 h. The protein precipitate was collected by centrifugation (14 000 rpm, 10 min) and resuspended in 0.1 M potassium phosphate buffer. To remove lipids and aggregates, an organic extraction was performed by addition of 1 volume of an n-BuOH/CHCl3 solution [1:1 (v/v)]. The VLP-containing aqueous layer was removed following centrifugation (14 000 rpm, 10 min), and particles were further purified by 10-40% sucrose gradient ultracentrifugation (25 000 rpm, 4 h). Visible-particle-containing bands were extracted, and particles were isolated by ultracentrifugation (68 000 rpm, 2 h). VLP pellets were decanted, resuspended in 0.1 M potassium phosphate buffer, and sterilized by 0.2 m PTFE syringe filters.

### Characterization of Qβ VLPs

### Fluorescent Labeling of Qβ VLPs

Qβ VLPs were fluorescently labeled using amine-reactive succinimidyl ester chemistry to conjugate either Alexa Fluor 488 NHS ester (0.1 mol) or Alexa Fluor 594 NHS ester (0.1 mol). Either Fluorescent NHS ester was added to a solution of Qβ VLPs (2 mg, 0.14 mol CP subunit), and after 4 h of gentle mixing at 4 °C. The reaction mixture was purified using a PD-10 desalting column, followed by concentrating using centrifugal filtration using an Amicon Ultra 100 k MW cutoff device.

### Comparative Proteomic Analysis

Generation of corona: Serum protein adsorption was conducted according to a modified method described previously(S. M. Pustulka, K. Ling, S. L. Pish, J. A. Champion, Protein nanoparticle charge and hydrophobicity govern protein corona and macrophage uptake. ACS Applied Materials & Interfaces 12, 48284-48295 (2020)). Thus, 100 μg of LP-01, PLGA, and SANG were resuspended in 250 μL of complete rat serum and incubated at RT for 3 h. A fourth sample of serum without particle was used as a control and was subject to the same conditions as the particles. The samples were then washed with two cycles of ultracentrifugation (160,000 g, 2 h) and resuspension in 250 uL DPBS to remove unbound proteins. The final samples were resuspended in 50 uL DPBS for analysis.

Proteolytic digestion: An aliquot of 1 μg/1 μL in 50 μL solutions of proteins was used for proteolytic digestion. Micro s-Trap (Protifi) columns were used for tryptic digestion as per the manufacturer's instructions. In brief 50 microgram dried protein were resuspended in 23 microliters of SDS lysis buffer. Proteins were reduced by adding TCEP (final concentration 5 mM TCEP) and incubating them at 55 °C for 15 minutes. Reduced samples were alkylated by adding Iodoacetamide at final concentration of 10 mM. Samples were acidified by adding phosphoric acid (final concentration ~2.5%). 165 microliters of binding buffer (100 mM TEAB (final) in 90% methanol) were added to each tube and the total contents were transferred to s-Trap columns. These tubes were spun at 4000 g for 1 minute and the flow through was discarded. Trapped proteins were washed three times with wash buffer (100 mM TEAB (final) in 90% methanol). Sequencing grade trypsin (Thermo Scientific) were added to the trap in a 1:20 trypsin to protein ratio in 30 microliters of 100 mM TEAB. The S-trap columns were incubated at 37 °C overnight. On the following day, digested peptides were eluted sequentially by 100 mM TEAB, 0.1% Formic acid and 50% Acetonitrile. Pooled eluates were dried in speedvac and samples were reconstituted in 25 microliters of 0.1% FA solution.

LC-MS/MS Data Acquisition and Analysis: An externally calibrated Thermo Q Exactive Plus (high-resolution electrospray tandem mass spectrometer) was used in conjunction with Dionex UltiMate3000 RSLCnano System. A 2 μL (1 g equivalent) sample was aspirated into a 50 μL loop and loaded onto the trap column (Thermo p-Precolumn 5 mm, with nanoViper tubing 30 μM i.d.×10 cm). The flow rate was set to 300 nL/min for separation on the analytical column (Acclaim pepmap RSLC 75 Mx 15 cm nanoviper). Mobile phase A was composed of 99.9% H2O (EMD Omni Solvent), and 0.1% formic acid and mobile phase B was composed of 99.9% ACN, and 0.1% formic acid. A 120-minute linear gradient from 3% to 45% B was performed. The LC eluent was directly nanosprayed into Q Exactive Plus mass spectrometer (Thermo Scientific). During the chromatographic separation, the Q Exactive plus was operated in a data-dependent mode and under direct control of the Thermo Excalibur 3.1.66 (Thermo Scientific). The MS data were acquired using the following parameters: 15 data-dependent collisional-induced-dissociation (CID) MS/MS scans per full scan (400 to 1800 m/z) at 70,000 resolutions. MS2 was acquired at 17,500 resolutions. Ions with single charge or charges more than 7 as well as unassigned charge were excluded. A 10 second dynamic exclusion window was used. All measurements were performed at room temperature.

Resultant Raw files were searched with Proteome Discoverer 3.0 using Sequest™ HT as the search engine using species specific FASTA with 10 ppm error tolerance. Percolator was used as peptide validator with 1% FDR (False Discovery Rate) as cut off.

### Statistical Analysis

Individual comparisons were tested for statistically significant differences with Bayesian one-way ANOVA (stan_glm)(J. Gabry, B. Goodrich, rstanarm: Bayesian applied regression modeling via Stan. R package version 2.18.1, (2018)). Bayesian parameters estimation derives the entire joint posterior distribution of all parameters simultaneously thus circumventing the need to correct for multiple tests on the data. Bayesian hypothesis testing was performed by analyzing the probability mass of the parameter region in question. This leads to a direct probability measure that defines values inside the 95% of the highest posterior density (HDI) more credible than outside values. HDI was used to make unbiased inferences by directly comparing the posterior probability distributions between two (or more) contrasts of interests e.g. mean-comparisons testing. For example, an HDI of a credible difference distribution that does not span zero indicates that the model predictions for the two conditions of interest are different from each other resulting in evidential support that parameters for both populations are unequal and are represented by (*)

All models were developed in fully hierarchal Bayesian framework with the rstanarm package (2.21.1)(J. Gabry, B. Goodrich, rstanarm: Bayesian applied regression modeling via Stan. R package version 2.18.1, (2018)) in the R environment (4.0.3). Rstanarm implements regression models in stan, which are fit using Hamiltonian Markov Chain Monte Carlo sampling to compute credible parameter values(S. Brooks, A. Gelman, G. Jones, X.-L. Meng, Handbook of markov chain monte carlo. (CRC press, 2011); M. D. Hoffman, A. Gelman, The No-U-turn sampler: adaptively setting path lengths in Hamiltonian Monte Carlo. Journal of Machine Learning Research 15, 1593-1623 (2014)). For intercepts and predictors, Student's t-distribution with mean zero and four degrees of freedom as the prior distribution was used. The scale of the prior distribution is 10 for the intercept and 2.5 for the predictors. Each model was run with four independent chains for 500 warm-up, 10,000 sampling steps and every second sample (thinning) was discarded. Steps to perform model evaluation and validation have been extensively described in our previous work. (S. N. Housley et al., Cancer Exacerbates Chemotherapy-Induced Sensory Neuropathy. Cancer Research, (2020); G. M. Horstman, S. N. Housley, T. C. Cope, Dysregulation of mechanosensory circuits coordinating the actions of antagonist motor pools following peripheral nerve injury and muscle reinnervation. Experimental neurology, (2019); S. N. Housley, P. Nardelli, R. K. Powers, M. M. Rich, T. C. Cope, Chronic defects in intraspinal mechanisms of spike encoding by spinal motoneurons following chemotherapy. Experimental Neurology, 113354 (2020); S. N. Housley, P. Nardelli, T. M. Rotterman, T. C. Cope, Neural circuit mechanisms of sensorimotor disability in cancer treatment. Proceedings of the National Academy of Sciences 118, (2021); T. M. Rotterman et al., Axon initial segment geometry in relation to motoneuron excitability. PloS one 16, e0259918 (2021)) Briefly, for all parameters, the number of effective samples was >2000, convergence was assessed and assumed to have reached the stationary distribution by ensuring that the Gelman-Rubin shrinkage statistic for all reported parameters was <1.05. Trace plots were examined and indicated clear stationarity and good mixing, and numerical checks of sampling quality indicated convergence.

### Example 4: Investigating SANGs as a Label Free Method for CTC Identification In Vitro

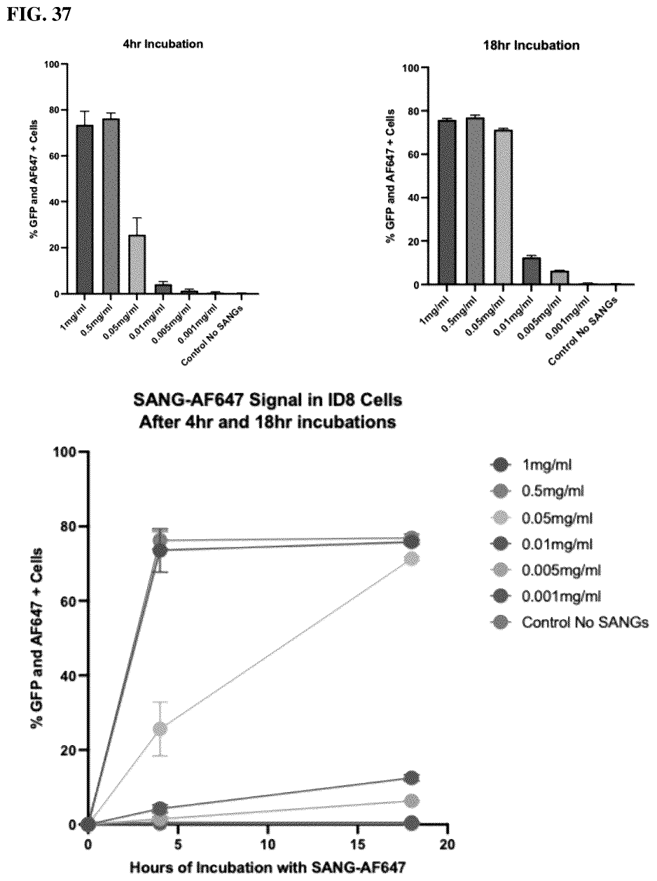

SANGs potential to detect cancer cells in vitro was then tested. Ovarian cancer cells were incubated with varying concentrations of SANGs for both 4 and 18 hours (FIG. 37) to determine the detection threshold of intracellular SANGs compared to other cellular populations. Following these concentration and time dependent in vitro experiments, samples were subjected to flow-cytometry based quantitative measures to determine the detection limits. Overall, it was determined that 0.5 mg/ml (~26 nM) SANGs concentrations were sufficient to sensitively label>70% of cancer cells. Longer in vitro exposure enables that optimal level to be reduced 10-fold to 0.05 mg/ml (~2.6 nM) SANGs concentrations.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

1. A method of detecting a solid tumor in a subject, the method comprising:
administering to the subject a composition comprising a population of nanohydrogels associated with an imaging agent;
wherein each of the population of nanohydrogels comprises a cross-linkable core/shell polymer;
wherein the nanohyrogel is configured to self-agglomerate in aberrant vasculature environments,
wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration;
after an incubation period, obtaining a biological sample from the subject; and
measuring a fluorescence signal of the biological sample ex vivo released by the imaging agent, wherein
the presence of a significant fluorescence signal indicates the presence of a solid tumor.

2. The method of claim 1, wherein the imaging agent is tethered to a core of the nanohydrogel.

3. The method of claim 1 or claim 2, wherein the crosslinkable core/shell polymer comprises a gel-forming polymer.

4. The method of claim 3, wherein the gel-forming polymer comprises a biocompatible polymer.

5. The method of any one of claims 1-4, wherein the crosslinkable core/shell polymer comprise polyacrylamide.

6. The method of any one of claims 1-5, wherein the crosslinkable core/shell polymer comprises a copolymer of N-isopropylmethacrylamide and N,N′-methylenebis(acrylamide).

7. The method of any one of claims 1-6, wherein a shell of the nanohydrogel comprises a cationic polymer ligand (e.g., poly(aminopropyl methacrylate)).

8. The method of any one of claims 1-7, wherein the crosslinkable core/shell polymer is unfunctionalized.

9. The method of any one of claims 1-8, wherein the nanohydrogel has a hydrodynamic size of from about 50 nm to about 250 nm prior to self-agglomeration.

10. The method of any one of claims 1-8, wherein the population of nanohydrogels is inducible via tortuous flow of an aberrant microvasculature of a tumor microenvironment (TME) to self-agglomerate to form an agglomerated cluster, wherein the agglomerated cluster has an average hydrodynamic size larger than an average hydrodynamic size prior to self-agglomeration.

11. The method of claim 10, wherein self-agglomeration of the population of nanohydrogels is concentration-dependent.

12. The method of any one of claims 10-11, wherein a concentration of nanohydrogels causing self-agglomeration is 10 nM or greater (e.g., 20 nM or greater, 30 nM or greater, 40 nM or greater, 50 nM or greater, 60 nM or greater, 70 nM or greater, 80 nM or greater, 90 nM or greater, 100 nM or greater, 110 nM or greater, 120 nM or greater, 130 nM or greater, 140 nM or greater, 150 nM or greater, 156 nM or greater, 200 nM or greater, 250 nM or greater, 500 nM or greater, 1 μM or greater).

13. The method of any one of claims 10-12, wherein a concentration of nanohydrogels causing self-agglomeration is from about 10 nM to about 1 μM (e.g., from about 10 nM to about 500 nM, from about 30 nM to about 500 nM, from about 60 nM to about 500 nM, from about 60 nM to about 250 nM, from about 100 nM to about 250 nM, from about 100 nM to about 200 nM, or about 156 nM).

14. The method of any one of claims 1-13, wherein a tumor selectivity of the nanohydrogel is 1% or greater (e.g., 1.5% or greater, 2% or greater, 2.5% or greater, 3% or greater, 3.5% or greater, 4% or greater, 4.5% or greater, 5% or greater, 5.5% or greater, 6% or greater, 6.5% or greater, 7% or greater, 7.5% or greater, 8% or greater, 9.5% or greater, 10% or greater, 10.5% or greater, 11% or greater, 11.5% or greater, 12% or greater, 12.5% or greater, 13% or greater, 13.5% or greater, 14% or greater, 14.5% or greater, 15% or greater, 20% or greater, or 25% or greater).

15. The method of any one of claims 1-14, wherein a ratio of relative delivery of the nanohydrogels between the tumor and a liver of the subject is 1:1 or more (e.g., 1.5:1 or more, 2:1 or more, 2.5:1 or more, 3:1 or more, 3.5:1 or more, 4:1 or more, 4.5:1 or more, 5:1 or more, 5.5:1 or more, 6:1 or more, 6.5:1 or more, 7:1 or more, 7.5:1 or more, 8:1 or more, 8.5:1 or more, 9:1 or more, 9.5:1 or more, 10:1 or more, 10.5:1 or more, 15:1 or more, 20:1 or more, or 25:1 or more).

16. The method of any one of claims 1-15, wherein 2.5% or more of the population of nanohydrogels is retained after 24 hours from an initial dose (e.g., 3% or more, 3.5% or more, 4% or more, 4.5% or more, 5% or more, 5.5% or more, 6% or more, 10% or more).

17. The method of any one of claims 1-16, wherein detecting comprises microscopy, barcode detection, scintillation counting, flow cytometry, or fluorescence-assisted cell sorting.

18. The method of any one of claims 1-17, wherein the imaging agent comprises a fluorophore.

19. The method of any one of claims 1-18, wherein the biological sample is a blood, plasma, or serum sample.

20. The method of any one of claims 1-19, wherein the solid tumor is ovarian cancer, colon cancer, lung cancer, breast cancer, or pancreatic cancer.

21. The method of claim 20, wherein the solid tumor is ovarian cancer.

22. A method of surgically debulking a solid tumor in a subject, comprising:
detecting the solid tumor in the subject, in vitro, according to the method of any one of claims 1-21;
imaging the solid tumor in the subject; and
surgically debulking the solid tumor.

23. The method of claim 22, wherein imaging the solid tumor comprises:
administering to the subject a composition comprising a population of nanohydrogels associated with an imaging agent;
wherein each of the population of nanohydrogels comprises a cross-linkable core/shell polymer;
wherein the nanohyrogel is configured to self-agglomerate in aberrant vasculature environments,
wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration, and
detecting the composition in the subject, thereby imaging the solid tumor.

24. A method of imaging a solid tumor in a subject, comprising:
administering to the subject a composition comprising a population of nanohydrogels associated with an imaging agent;
wherein each of the population of nanohydrogels comprises a cross-linkable core/shell polymer;
wherein the nanohyrogel is configured to self-agglomerate in aberrant vasculature environments,
wherein the population of nanohydrogels is inducible via tortuous flow of an aberrant microvasculature of a tumor microenvironment (TME) to self-aggregate to form an agglomerated cluster, wherein the agglomerated cluster has an average hydrodynamic size larger than an average hydrodynamic size prior to self-agglomeration, and
detecting the composition in the subject, thereby imaging the solid tumor.

25. The method of claim 24, wherein the agent is tethered to a core of the nanohydrogel.

26. The method of claim 24 or claim 25, wherein the crosslinkable core/shell polymer comprises a gel-forming polymer.

27. The method of claim 26, wherein the gel-forming polymer comprises a biocompatible polymer.

28. The method of any one of claims 24-27, wherein the crosslinkable core/shell polymer comprise polyacrylamide.

29. The method of any one of claims 24-28, wherein the crosslinkable core/shell polymer comprises a copolymer of N-isopropylmethacrylamide and N,N′-methylenebis(acrylamide).

30. The method of any one of claims 24-29, wherein a shell of the nanohydrogel comprises a cationic polymer ligand (e.g., poly(aminopropyl methacrylate)).

31. The method of any one of claims 24-30, wherein the crosslinkable core/shell polymer is unfunctionalized.

32. The method of any one of claims 24-31, wherein the nanohydrogel has a hydrodynamic size of from about 50 nm to about 250 nm prior to self-agglomeration.

33. The method of any one of claims 24-31, wherein the population of nanohydrogels is inducible via tortuous flow of an aberrant microvasculature of a tumor microenvironment (TME) to self-agglomerate to form an agglomerated cluster, wherein the agglomerated cluster has an average hydrodynamic size larger than an average hydrodynamic size prior to self-agglomeration.

34. The method of claim 33, wherein self-agglomeration of the population of nanohydrogels is concentration-dependent.

35. The method of any one of claims 33-34, wherein a concentration of nanohydrogels causing self-agglomeration is 10 nM or greater (e.g., 20 nM or greater, 30 nM or greater, 40 nM or greater, 50 nM or greater, 60 nM or greater, 70 nM or greater, 80 nM or greater, 90 nM or greater, 100 nM or greater, 110 nM or greater, 120 nM or greater, 130 nM or greater, 140 nM or greater, 150 nM or greater, 156 nM or greater, 200 nM or greater, 250 nM or greater, 500 nM or greater, 1 μM or greater).

36. The method of any one of claims 33-35, wherein a concentration of nanohydrogels causing self-agglomeration is from about 10 nM to about 1 μM (e.g., from about 10 nM to about 500 nM, from about 30 nM to about 500 nM, from about 60 nM to about 500 nM, from about 60 nM to about 250 nM, from about 100 nM to about 250 nM, from about 100 nM to about 200 nM, or about 156 nM).

37. The method of any one of claims 24-36, wherein a tumor selectivity of the nanohydrogel is 1% or greater (e.g., 1.5% or greater, 2% or greater, 2.5% or greater, 3% or greater, 3.5% or greater, 4% or greater, 4.5% or greater, 5% or greater, 5.5% or greater, 6% or greater, 6.5% or greater, 7% or greater, 7.5% or greater, 8% or greater, 9.5% or greater, 10% or greater, 10.5% or greater, 11% or greater, 11.5% or greater, 12% or greater, 12.5% or greater, 13% or greater, 13.5% or greater, 14% or greater, 14.5% or greater, 15% or greater, 20% or greater, or 25% or greater).

38. The method of any one of claims 24-37, wherein a ratio of relative delivery of the nanohydrogels between the tumor and a liver of the subject is 1:1 or more (e.g., 1.5:1 or more, 2:1 or more, 2.5:1 or more, 3:1 or more, 3.5:1 or more, 4:1 or more, 4.5:1 or more, 5:1 or more, 5.5:1 or more, 6:1 or more, 6.5:1 or more, 7:1 or more, 7.5:1 or more, 8:1 or more, 8.5:1 or more, 9:1 or more, 9.5:1 or more, 10:1 or more, 10.5:1 or more, 15:1 or more, 20:1 or more, or 25:1 or more).

39. The method of any one of claims 24-38, wherein 2.5% or more of the population of nanohydrogels is retained after 24 hours from an initial dose (e.g., 3% or more, 3.5% or more, 4% or more, 4.5% or more, 5% or more, 5.5% or more, 6% or more, 10% or more).

40. The method of any one of claims 24-39, wherein the imaging agent comprises a contrast agent, a heavy metal, or a radioisotope.

41. The method of any one of claims 24-40, wherein detecting comprises positron emission tomography.

42. The method of any one of claims 24-41, wherein detecting the composition in the subject occurs while the subject is undergoing a surgical procedure.

43. The method of claim 42, further comprising surgically debulking the solid tumor after detecting the composition in the subject.

44. The method of claim 43, wherein the surgically debulking the solid tumor comprises surgically removing at least 80% of the solid tumor, surgically removing at least 90% of the solid tumor, or surgically removing at least 95% of the solid tumor.

45. The method of any one of claims 24-44, wherein the solid tumor is ovarian cancer, colon cancer, lung cancer, breast cancer, or pancreatic cancer.
